(19) United States
(12) Patent Application Publication
(10) Pub. No.: US 2025/0155806 A1
(43) Pub. Date: May 15, 2025

(54) RESIST COMPOSITION, METHOD FOR FORMING RESIST PATTERN, COMPOUND, AND ACID GENERATING AGENT

(71) Applicant: TOKYO OHKA KOGYO CO., LTD., Kawasaki-shi (JP)

(72) Inventors: Makoto SATO, Kawasaki-shi (JP); Takayoshi MORI, Kawasaki-shi (JP); Ryohei EGUCHI, Kawasaki-shi (JP); Kazuyuki NITTA, Kawasaki-shi (JP)

(21) Appl. No.: 18/937,982

(22) Filed: Nov. 5, 2024

(30) Foreign Application Priority Data
Nov. 10, 2023 (JP) 2023-192592
May 24, 2024 (JP) 2024-085115

(51) Int. Cl. G03F 7/038; G03F 7/004; G03F 7/039

(52) U.S. Cl. CPC G03F 7/0382; G03F 7/0045; G03F 7/0048; G03F 7/0397

(57) ABSTRACT

A resist composition that generates an acid upon exposure and whose solubility with respect to a liquid developer changes by an action of the acid. The resist composition includes a base material component whose solubility with respect to a liquid developer changes by an action of an acid; an acid generating agent component that generates an acid upon exposure; and an acid diffusion control agent component, in which the acid generating agent component includes a compound represented by a general formula (b1-1)

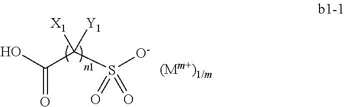

### CROSS-REFERENCE TO RELATED APPLICATIONS

The present application is based on Japanese Patent Application No. 2023-192592 filed on Nov. 10, 2023 and Japanese Patent Application No. 2024-085115 filed on May 24, 2024, the entire contents of which are hereby incorporated by reference.

### TECHNICAL FIELD

The present invention relates to a resist composition, a method for forming a resist pattern, a compound, and an acid generating agent.

### BACKGROUND ART

In a lithography technique, for example, a process is performed in which a resist film made of a resist material is formed on a substrate, the resist film is selectively exposed, and a developing treatment is performed to form a resist pattern having a predetermined shape on the resist film. A resist material whose property changes such that an exposed portion of a resist film is dissolved in a liquid developer is referred to as a positive resist material, and a resist material whose property changes such that an exposed portion is not dissolved in a liquid developer is referred to as a negative resist material.

In recent years, in the manufacture of semiconductor elements and liquid crystal display elements, miniaturization of patterns has been more rapidly advanced with the progress of the lithography technique.

A common miniaturization technique is wavelength shortening (energy increase) of a light source for exposure. Specifically, in the related art, ultraviolet rays such as g-line and i-line have been used, but currently mass production of semiconductor elements using KrF excimer lasers and ArF excimer lasers has begun. Studies have been conducted on extreme ultraviolet (EUV), electron beam (EB), X-ray, and the like having a wavelength shorter (energy higher) than that of the excimer lasers.

In such a situation, the resist material is required to have lithography properties such as a sensitivity to these exposure light sources or energy sources and resolution capable of reproducing a pattern with a fine dimension.

As a resist material satisfying such requirements, in the related art, a chemically amplified resist composition containing an acid generating agent component that generates an acid upon exposure and a base material component whose solubility with respect to a liquid developer changes by an action of the acid has been used.

For example, in Patent Literature 1, a resist composition and a method for forming a resist pattern which can achieve a high sensitivity and have excellent lithography properties are studied. Patent Literature 1 describes a resist composition in which a plurality of kinds of acid generating agent components each having a specific structure is used and a macromolecular compound having a plurality of structural units each having a specific structure is employed to improve a dissociation performance with respect to acid.

### CITATION LIST

### Patent Literature

Patent Literature 1: WO2017/065207

### SUMMARY OF THE INVENTION

As the lithography technique continues to advance and the miniaturization of resist patterns is further advanced, a resist composition is required to have good lithography properties such as a high sensitivity to an exposure light source and reduced roughness.

Further, in recent years, from the viewpoint of environmental protection, there has been a demand for reducing the use of organic fluorine compounds.

However, when fluorine atoms are removed from a fluorine-containing compound used as an acid generating agent, a good pattern shape can be obtained, but the sensitivity tends to deteriorate, and there is a trade-off between the pattern shape and the sensitivity.

The present invention has been made in consideration of the above-described circumstances, and an object of the present invention is to provide a compound, an acid generating agent, a resist composition, and a method for forming a resist pattern, which are capable of reducing an environmental burden, achieving a high sensitivity, and forming a resist pattern having excellent lithography properties.

As a result of intensive studies to solve the above problems, the present inventors have found that a compound, an acid generating agent, a resist composition, and a method for forming a resist pattern, which are capable of reducing an environmental burden, achieving a high sensitivity, and forming a resist pattern having excellent lithography properties can be obtained by the following configuration, and have completed the present invention.

That is, the present invention is as follows.

A resist composition according to an embodiment of the present invention is a resist composition that generates an acid upon exposure and whose solubility with respect to a liquid developer changes by an action of the acid, the resist composition including:
a base material component (A) whose solubility with respect to a liquid developer changes by an action of an acid;
an acid generating agent component (B) that generates an acid upon exposure; and
an acid diffusion control agent component (D), in which
the acid generating agent component (B) includes a compound represented by the following general formula (b1-1).

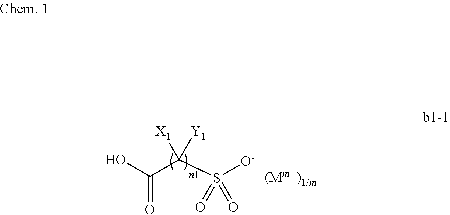

In the general formula (b1-1), X₁ and Y₁ each independently represent a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group; X₁ and Y₁ may be bonded to each other to form a ring; n1 represents an integer of 1 to 10; m is an integer of 1 or more; and M^{m+} represents an m-valent organic cation.

A method for forming a resist pattern according to the second embodiment of the present invention is a method for forming a resist pattern including:
forming a resist film on a support using the resist composition according to the resist composition according to the embodiment of the present invention;
exposing the resist film; and
developing the resist film to form a resist pattern.

A compound according to the third embodiment of the present invention is represented by the general formula (b1-1).

An acid generating agent according to the fourth embodiment of the present invention includes:
a compound represented by the general formula (b1-1).

According to the present invention, a compound, an acid generating agent, a resist composition, and a method for forming a resist pattern, which are capable of reducing an environmental burden, achieving a high sensitivity, and forming a resist pattern having excellent lithography properties can be provided.

### DESCRIPTION OF EMBODIMENTS

Hereinafter, embodiments of the present invention will be described in detail. The present invention is not limited to the embodiments described below.

In the present disclosure, the term “aliphatic” is a relative concept to aromatic and is defined as meaning a group or a compound that does not have aromaticity.

Unless otherwise specified, the term “alkyl group” includes a linear monovalent saturated hydrocarbon group, a branched monovalent saturated hydrocarbon group, and a cyclic monovalent saturated hydrocarbon group. The same applies to an alkyl group in an alkoxy group.

Unless otherwise specified, the term “alkylene group” includes a linear divalent saturated hydrocarbon group, a branched divalent saturated hydrocarbon group, and a cyclic divalent saturated hydrocarbon group.

The term “halogenated alkyl group” is a group in which a part or all of hydrogen atoms in an alkyl group are replaced by halogen atoms, and examples of the halogen atom include a fluorine atom, a chlorine atom, a bromine atom, and an iodine atom.

The term “fluorinated alkyl group” or “fluorinated alkylene group” refers to a group in which a part or all of hydrogen atoms in an alkyl group or alkylene group are replaced by fluorine atoms.

The term “structural unit” refers to a monomer unit that constitutes a macromolecular compound (a resin, a polymer, or a copolymer).

The expression “may include a substituent” includes both a case where a hydrogen atom (—H) is replaced by a monovalent group and a case where a methylene group (—CH₂—) is replaced by a divalent group.

The term “exposure” is intended to include a general concept of irradiation with radiation rays.

The expression “structural unit derived from an acrylic ester” means a structural unit formed by cleavage of an ethylenic double bond of an acrylic ester.

The “acrylic ester” is a compound in which a hydrogen atom at a terminal carboxy group of acrylic acid (CH₂═CH—COOH) is replaced by an organic group.

In the acrylic ester, a hydrogen atom bonded to a carbon atom at an α-position may be replaced by a substituent. A substituent (R^{α0}) for replacing the hydrogen atom bonded to the carbon atom at the α-position is an atom other than a hydrogen atom or a group, and examples thereof include an alkyl group having 1 to 5 carbon atoms and a halogenated alkyl group having 1 to 5 carbon atoms. The acrylic ester also includes an itaconic acid diester in which the substituent (R^{α0}) is replaced by a substituent including an ester bond, and an α-hydroxy acrylic ester in which the substituent (R^{α0}) is replaced by a hydroxyalkyl group or a group modified with a hydroxy group thereof. Unless otherwise specified, the carbon atom at the α-position of the acrylic ester refers to a carbon atom to which a carbonyl group of acrylic ester is bonded.

Hereinafter, an acrylic ester in which the hydrogen atom bonded to the carbon atom at the α-position is replaced by a substituent may be referred to as an α-replaced acrylic ester. In addition, the acrylic ester and the α-replaced acrylic ester may be collectively referred to as “(α-replaced) acrylic ester”.

The term “structural unit derived from hydroxystyrene” refers to a structural unit formed by cleavage of an ethylenic double bond of hydroxystyrene. The term “structural unit derived from a hydroxystyrene derivative” refers to a structural unit formed by cleavage of an ethylenic double bond of a hydroxystyrene derivative.

The term “hydroxystyrene derivative” is a concept including those in which a hydrogen atom at the α-position of hydroxystyrene is replaced by another substituent such as an alkyl group or a halogenated alkyl group, and derivatives thereof. Examples of the derivative include those in which a hydrogen atom of a hydroxy group of hydroxystyrene in which a hydrogen atom at the α-position may be replaced by a substituent is replaced by an organic group; and those in which a substituent other than a hydroxy group is bonded to a benzene ring of hydroxystyrene in which a hydrogen atom at the α-position may be replaced by a substituent. Unless otherwise specified, the α-position (the carbon atom at the α-position) refers to a carbon atom to which a benzene ring is bonded.

Examples of the substituent for replacing the hydrogen atom of the hydroxystyrene at the α-position include the same as those described for the substituent at the α-position in the above-described α-substituted acrylic ester.

The term “structural unit derived from vinylbenzoic acid or a vinylbenzoic acid derivative” refers to a structural unit formed by cleavage of an ethylenic double bond of vinylbenzoic acid or a vinylbenzoic acid derivative.

The term “vinylbenzoic acid derivative” is a concept including those in which a hydrogen atom of vinylbenzoic acid at the α-position is replaced by another substituent such as an alkyl group or a halogenated alkyl group, and derivatives thereof. Examples of the derivative include those in which a hydrogen atom of a carboxy group of vinylbenzoic acid in which a hydrogen atom at the α-position may be replaced by a substituent is replaced by an organic group; and those in which a substituent other than a hydroxy group or a carboxy group is bonded to a benzene ring of vinylbenzoic acid in which a hydrogen atom at the α-position may be replaced by a substituent. Unless otherwise specified, the α-position (the carbon atom at the α-position) refers to a carbon atom to which a benzene ring is bonded.

An alkyl group as the substituent at the α-position is preferably a linear or branched alkyl group, and specific examples thereof include an alkyl group having 1 to 5 carbon atoms (a methyl group, an ethyl group, a propyl group, an isopropyl group, an n-butyl group, an isobutyl group, a tert-butyl group, a pentyl group, an isopentyl group, and a neopentyl group).

Specific examples of a halogenated alkyl group as the substituent at the α-position include a group in which a part or all of hydrogen atoms in the “alkyl group as the substituent at the α-position” are replaced by halogen atoms. Examples of the halogen atom include a fluorine atom, a chlorine atom, a bromine atom, and an iodine atom. A fluorine atom is particularly preferred.

Specific examples of a hydroxyalkyl group as the substituent at the α-position include a group in which a part or all of hydrogen atoms in the “alkyl group as the substituent at the α-position” are replaced by hydroxy groups. The number of hydroxy groups in the hydroxyalkyl group is preferably 1 to 5, and most preferably 1.

In the present disclosure, a numerical range indicated using the symbol “-” or word “to” means a range that includes numerical values written before and after the symbol “-” or word “to” as the lower limit value and the upper limit value, respectively.

Further, in the present disclosure, when a plurality of substances corresponding to each component are present in a composition, an amount of each component in the composition means a total amount of a plurality of corresponding substances present in the composition, unless otherwise specified.

A chemical structural formula in the present disclosure may be described as a simplified structural formula in which hydrogen atoms are omitted.

In the present disclosure, a chiral carbon may exist and hence enantiomers or diastereomers may exist depending on a structure of a chemical formula. In that case, one chemical formula represents all the isomers. The isomers may be used alone or as a mixture.

In the present disclosure, “mass %” and “weight %” have the same meaning, and “parts by mass” and “parts by weight” have the same meaning.

### Resist Composition

A resist composition according to an embodiment of the present invention is a resist composition that generates an acid upon exposure and whose solubility with respect to a liquid developer changes by an action of the acid. The resist composition includes a base material component (A) (hereinafter, also referred to as “component (A)”) whose solubility with respect to a liquid developer changes by an action of an acid, an acid generating agent component (B) (hereinafter, also referred to as “component (B)”) that generates an acid upon exposure, and an acid diffusion control agent component (D) (hereinafter, also referred to as “component (D)”). The acid generating agent component (B) includes a compound represented by the general formula (b1-1).

When a resist film is formed using the resist composition of the present embodiment and the resist film is selectively exposed, an acid is generated in an exposed portion of the resist film, and a solubility of the component (A) with respect to a liquid developer changes by an action of the acid, whereas the solubility of the component (A) with respect to the liquid developer does not change in an unexposed portion of the resist film, resulting in a difference in solubility with respect to the liquid developer between the exposed portion and the unexposed portion of the resist film. Therefore, when the resist film is developed, the exposed portion of the resist film is dissolved and removed to form a positive resist pattern in the case where the resist composition is a positive resist composition, and the unexposed portion of the resist film is dissolved and removed to form a negative resist pattern in the case where the resist composition is a negative resist composition.

In the present disclosure, a resist composition that forms a positive resist pattern by dissolving and removing an exposed portion of a resist film is referred to as a positive resist composition, and a resist composition that forms a negative resist pattern by dissolving and removing an unexposed portion of a resist film is referred to as a negative resist composition.

The resist composition of the present embodiment may be a positive resist composition or a negative resist composition.

The resist composition of the present embodiment may be for an alkaline developing process in which an alkaline liquid developer is used in a developing treatment at the time of resist pattern formation, or may be for a solvent developing process in which a liquid developer (an organic liquid developer) containing an organic solvent is used in the developing treatment.

That is, the resist composition of the present embodiment is a “positive resist composition for an alkaline developing process” that forms a positive resist pattern in an alkaline developing process, and is a “negative resist composition for a solvent developing process” that forms a negative resist pattern in a solvent developing process.

The resist composition of the present embodiment is a resist composition that generates an acid upon exposure and whose solubility with respect to a liquid developer changes by an action of the acid. The resist composition contains the base material component (A) whose solubility with respect to a liquid developer changes by an action of an acid, the acid generating agent component (B) that generates an acid upon exposure, and the acid diffusion control agent component (D).

The component (A) may generate an acid upon exposure, and in this case, the component (A) is a “base material component that generates an acid upon exposure and whose solubility with respect to a liquid developer changes by an action of the acid”. When the component (A) is a base material component that generates an acid upon exposure and whose solubility with respect to a liquid developer changes by an action of the acid, a component (A1) to be described later is preferably a macromolecular compound that generates an acid upon exposure and whose solubility with respect to a liquid developer changes by an action of the acid. As such a macromolecular compound, a copolymer including a structural unit that generates an acid upon exposure can be used. Examples of the structural unit that generates an acid upon exposure include known structural units.

The resist composition according to the embodiment of the present invention contains a compound represented by a general formula (b1-1) to be described later as the acid generating agent component (B).

In a resist process in related art, a fluorine-containing PAG anion, which is generally used as an acid generating agent, has a problem that the fluorine-containing PAG anion segregates on the surface of the resist pattern, deteriorating rectangularity of a pattern shape. However, when fluorine is removed from the fluorine-containing PAG anion, the rectangularity of the pattern shape is improved, but a sensitivity becomes very low.

Since the compound represented by the general formula (b1-1) according to the embodiment of the present invention contains a sulfonate anion having a chain carboxylic acid, hydrogen of the carboxylic acid serves as a hydrogen bond donating site and interacts with the sulfonic acid, whereby the anion is stabilized, and as a result, the pKa decreases, and the sensitivity is increased. Further, it is presumed that since the compound represented by the general formula (b1-1) does not contain a fluorine atom, not only the environment burden can be reduced, but also the segregation of the PAG anion on the resist pattern surface can be eliminated, and a resist pattern having a good shape can be formed.

### <<Component (A)>>

In the resist composition of the present embodiment, the component (A) is a base material component whose solubility with respect to the liquid developer changes by the action of the acid, and the component (A) preferably contains a resin component (A1) (hereinafter, also referred to as “component (A1)”) whose solubility with respect to the liquid developer changes by the action of the acid. The component (A) may be one whose solubility with respect to the liquid developer increases by the action of the acid, or one whose solubility with respect to the liquid developer decreases by the action of the acid. By using the component (A1), a polarity of the base material component (A) changes before and after the exposure, so that a good development contrast can be obtained not only in the alkaline developing process but also in the solvent developing process.

As the component (A), at least the component (A1) is used, and other macromolecular compounds and/or low molecular compounds may be used in combination with the component (A1).

When the alkali developing process is applied, the base material component (A) containing the component (A1) is poorly soluble with respect to the alkaline liquid developer before exposure, and when an acid is generated from the component (B) upon exposure, the polarity of the base material component (A) increases by the action of the acid, thereby increasing the solubility with respect to the alkaline liquid developer. Therefore, in forming a resist pattern, when a resist film obtained by coating a support with the resist composition is selectively exposed, an exposed portion of the resist film changes from poorly soluble to soluble with respect to the alkaline liquid developer, whereas an unexposed portion of the resist film remains poorly soluble with respect to alkali and does not change. Therefore, a positive resist pattern can be formed by alkaline development.

On the other hand, when the solvent developing process is applied, the base material component (A) containing the component (A1) has a high solubility with respect to the organic liquid developer before exposure, and when an acid is generated from the component (B) upon exposure, the polarity of the base material component (A) increases by the action of the acid, and the solubility with respect to the organic liquid developer decreases. Therefore, in forming a resist pattern, when a resist film obtained by coating a support with the resist composition is selectively exposed, an exposed portion of the resist film changes from soluble to poorly soluble with respect to the organic liquid developer, whereas an unexposed portion of the resist film remains soluble and does not change. Therefore, by developing with the organic liquid developer, a contrast can be created between the exposed portion and the unexposed portion, and a negative resist pattern can be formed.

As the component (A), a component containing the macromolecular compound (A1) (hereinafter, also referred to as “component (A1)”) having a structural unit (a1) containing an acid-decomposable group whose polarity increases by the action of the acid is preferred.

As the component (A1), a macromolecular compound having the structural unit (a1) and a structural unit (a2) containing a lactone-containing cyclic group, a —SO₂-containing cyclic group, or a carbonate-containing cyclic group is preferably used.

In the resist composition according to the embodiment of the present invention, the component (A) may be used alone or in combination of two or more kinds thereof.

The component (A) is preferably a macromolecular compound that does not fall under the Per- and Polyfluoroalkyl Substances (PFAS). That is, the component (A1) is preferably a compound that does not fall under the PFAS. When the component (A) contains other components in addition to the component (A1), it is preferable that the other components are also compounds that do not fall under the PFAS. The term “PFAS” refers to a compound containing a trifluoromethyl group (—CF₃) and a compound containing a difluoromethylene group (—CF₂—).

<Structural Unit (a1)>

The structural unit (a1) is a structural unit containing an acid-decomposable group whose polarity increases by the action of the acid.

The “acid-decomposable group” is a group having an acid-decomposable property in which at least a part of bonds in the structure of the acid-decomposable group can be cleaved by the action of the acid.

Examples of the acid-decomposable group whose polarity increases by the action of the acid include a group that is decomposed by the action of the acid to generate a polar group.

Examples of the polar group include a carboxy group, a hydroxy group, an amino group, and a sulfo group (—SO₃H). Among them, a polar group containing —OH in the structure (hereinafter, may be referred to as an “OH-containing polar group”) is preferred, a carboxy group or a hydroxy group is more preferred, and a carboxy group is particularly preferred.

Specific examples of the acid-decomposable group include a group in which the polar group is protected with an acid-dissociable group (for example, a group in which a hydrogen atom of an OH-containing polar group is protected with an acid-dissociable group).

Here, the term “acid-dissociable group” refers to both (i) a group having an acid dissociable property in which a bond between the acid-dissociable group and an atom adjacent to the acid-dissociable group can be cleaved by an action of an acid, and (ii) a group in which a bond between the acid-dissociable group and an atom adjacent to the acid dissociable-group can be cleaved by further causing a decarboxylation reaction after a part of bonds is cleaved by the action of the acid.

The acid-dissociable group constituting the acid-decomposable group needs to be a group having a polarity lower than that of a polar group generated by dissociation of the acid-dissociable group. Accordingly, when the acid-dissociable group is dissociated by the action of the acid, a polar group having a polarity higher than that of the acid-dissociable group is generated, thereby increasing the polarity. As a result, the polarity of the entire component (A1) increases. When the polarity increases, the solubility with respect to the liquid developer relatively changes, the solubility increases when the liquid developer is an alkaline liquid developer, and the solubility decreases when the liquid developer is an organic liquid developer.

The structural unit (a1) preferably contains an acid-decomposable group having an alicyclic hydrocarbon group, and more preferably contains an acid-decomposable group having a monocyclic alicyclic hydrocarbon group.

Since the acid-decomposable group (the acid-dissociable group) in the structural unit (a1) has an appropriate bulkiness, the acid diffusion control and the solubility with respect to the liquid developer can be appropriately adjusted, and the roughness in forming a resist pattern can be reduced.

Examples of the acid-dissociable group in the structural unit (a1) include those proposed as acid-dissociable groups for base resins for chemically amplified resists in the related art.

Specific examples of those proposed as acid-dissociable groups for base resins for chemically amplified resist compositions include an “acetal acid-dissociable group”, a “tertiary alkyl ester acid-dissociable group” and a “tertiary alkyloxycarbonyl acid-dissociable group”.

### Acetal Acid-Dissociable Group:

Among the polar groups, examples of the acid-dissociable group that protects a carboxy group or a hydroxy group include an acid-dissociable group represented by the following formula (a1-r-1) (hereinafter, may be referred to as an “acetal acid-dissociable group”).

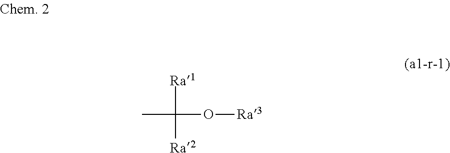

In the formula, Ra′¹ and Ra′² are a hydrogen atom or an alkyl group. Ra′³ is a hydrocarbon group, and Ra′³ may be bonded to either Ra′¹ or Ra′² to form a ring.

In the formula (a1-r-1), it is preferable that at least one of Ra′¹ and Ra′² be a hydrogen atom, and it is more preferable that both be a hydrogen atom.

When Ra′¹ or Ra′² is an alkyl group, examples of the alkyl group include those same as the alkyl group described as the substituent that may be bonded to the carbon atom at the α-position in the description of the above α-substituted acrylic ester, and an alkyl group having 1 to 5 carbon atoms is preferred. Specifically, preferred examples thereof include a linear or branched alkyl group. More specific examples thereof include a methyl group, an ethyl group, a propyl group, an isopropyl group, an n-butyl group, an isobutyl group, a tert-butyl group, a pentyl group, an isopentyl group, and a neopentyl group. A methyl group or an ethyl group is more preferred, and a methyl group is particularly preferred.

In the formula (a1-r-1), examples of the hydrocarbon group for Ra′³ include a linear or branched alkyl group, or a cyclic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferred, and a methyl group or an ethyl group is more preferred.

The branched alkyl group preferably has 3 to 10 carbon atoms, and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. An isopropyl group is preferred.

When Ra′³ is a cyclic hydrocarbon group, the hydrocarbon group may be an aliphatic hydrocarbon group or an aromatic hydrocarbon group, and may be a polycyclic group or a monocyclic group.

The aliphatic hydrocarbon group which is a monocyclic group is preferably a group in which one hydrogen atom is removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom is removed from a polycycloalkane. The polycycloalkane is preferably a group having 7 to 12 carbon atoms, and specific examples thereof include adamantane, norbornane, isobornane, tricyclodecane, and tetracyclododecane.

When the cyclic hydrocarbon group for Ra′³ is an aromatic hydrocarbon group, the aromatic hydrocarbon group is a hydrocarbon group having at least one aromatic ring.

The aromatic ring is not particularly limited as long as it is of a cyclic conjugated system having 4n+2 π electrons, and may be monocyclic or polycyclic. The number of carbon atoms of the aromatic ring is preferably 5 to 30, more preferably 5 to 20, still more preferably 6 to 15, and particularly preferably 6 to 12.

Specific examples of the aromatic ring include an aromatic hydrocarbon ring such as benzene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Specific examples of the aromatic hydrocarbon group in Ra′³ include a group in which one hydrogen atom is removed from the aromatic hydrocarbon ring or aromatic heterocyclic ring (an aryl group or a heteroaryl group); a group in which one hydrogen atom is removed from an aromatic compound containing two or more aromatic rings (for example, biphenyl or fluorene); and a group in which one hydrogen atom in the aromatic hydrocarbon ring or aromatic heterocyclic ring is replaced by an alkylene group (for example, an arylalkyl group such as a benzyl group, a phenethyl group, a 1-naphthyl methyl group, a 2-naphthyl methyl group, a 1-naphthyl ethyl group, or a 2-naphthyl ethyl group). The number of carbon atoms of the alkylene group bonded to the aromatic hydrocarbon ring or aromatic heterocyclic ring is preferably 1 to 4, more preferably 1 to 2, and particularly preferably 1.

The cyclic hydrocarbon group in Ra′³ may include a substituent. Examples of the substituent include —R^{P1}, —R^{P2}—O—R^{P1}, —R^{P2}—CO—R^{P1}, —R^{P2}—CO—OR^{P1}, —R^{P2}—O—CO—R^{P1}, —R^{P2}—OH, —R^{P2}—CN, and —R^{P2}—COOH (hereinafter, these substituents are collectively referred to as “Ra⁰⁵”).

Here, R^{P1} is a monovalent chain saturated hydrocarbon group having 1 to 10 carbon atoms, a monovalent aliphatic cyclic saturated hydrocarbon group having 3 to 20 carbon atoms, or a monovalent aromatic hydrocarbon group having 6 to 30 carbon atoms. R^{P2} is a single bond, a divalent chain saturated hydrocarbon group having 1 to 10 carbon atoms, a divalent aliphatic cyclic saturated hydrocarbon group having 3 to 20 carbon atoms, or a divalent aromatic hydrocarbon group having 6 to 30 carbon atoms. Part or all of hydrogen atoms of the chained saturated hydrocarbon group, the aliphatic cyclic saturated hydrocarbon group, and the aromatic hydrocarbon group for R^{P1} and R^{P2} may be replaced by fluorine atoms. The aliphatic cyclic hydrocarbon group may have one or more substituents of one kind mentioned above, or may have one or more substituents of each of two or more kinds mentioned above.

Examples of the monovalent chain saturated hydrocarbon group having 1 to 10 carbon atoms include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, and an adamantyl group.

Examples of the monovalent aromatic hydrocarbon group having 6 to 30 carbon atoms include a group in which one hydrogen atom is removed from an aromatic hydrocarbon ring such as benzene, biphenyl, fluorene, naphthalene, anthracene, and phenanthrene.

When Ra′³ is bonded to either Ra′¹ or Ra′² to form a ring, the cyclic group is preferably a 4- to 7-membered ring, and more preferably a 4- to 6-membered ring. Specific examples of the cyclic group include a tetrahydropyranyl group and a tetrahydrofuranyl group.

### Tertiary Alkyl Ester Acid-Dissociable Group:

Among the polar groups, examples of the acid-dissociable group that protects a carboxy group include an acid-dissociable group represented by the following general formula (a1-r-2).

Among the acid-dissociable group represented by the following formula (a1-r-2), those constituted by an alkyl group may be referred to as a “tertiary alkyl ester acid-dissociable group” hereinafter for convenience.

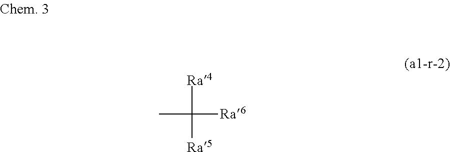

In the formula, Ra′⁴ to Ra′⁶ each represent a hydrocarbon group, and Ra′⁵ and Ra′⁶ may be bonded to each other to form a ring.]

Examples of the hydrocarbon group represented by Ra′⁴ include a linear or branched alkyl group, a chain or cyclic alkenyl group, or a cyclic hydrocarbon group.

Examples of the linear or branched alkyl group and the cyclic hydrocarbon group (the aliphatic hydrocarbon group which is a monocyclic group, the aliphatic hydrocarbon group which is a polycyclic group, and the aromatic hydrocarbon group) in Ra′⁴ include the same as the linear or branched alkyl group and cyclic hydrocarbon group in Ra′³.

The chain or cyclic alkenyl group in Ra′⁴ is preferably an alkenyl group having 2 to 10 carbon atoms.

Examples of the hydrocarbon group for Ra′⁵ and Ra′⁶ include the same as the hydrocarbon group for Ra′³.

When Ra′⁵ and Ra′⁶ are bonded to each other to form a ring, suitable examples of the acid-dissociable group represented by the above formula (a1-r-2) include a group represented by the following formula (a1-r2-1), a group represented by the following formula (a1-r2-2), and a group represented by the following formula (a1-r2-3).

On the other hand, when Ra′⁴ to Ra′⁶ are not bonded to each other and are independent a hydrocarbon group, suitable examples of the acid-dissociable group represented by the above formula (a1-r-2) include a group represented by the following formula (a1-r2-4).

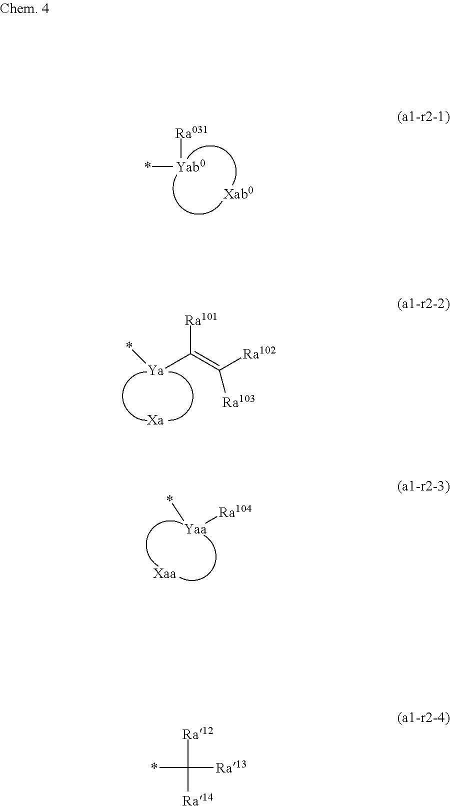

In the formula (a1-r2-1), Ra⁰³¹ represents an alkyl group, and Yab⁰ represents a carbon atom. Xab⁰ represents a group that forms an alicyclic hydrocarbon group together with Yab⁰, and part or all of hydrogen atoms of the alicyclic hydrocarbon group may be replaced. In the formula (a1-r2-2), Ya represents a carbon atom. Xa is a group that forms a cyclic hydrocarbon group together with Ya. Part or all of hydrogen atoms of the cyclic hydrocarbon group may be replaced. Ra¹⁰¹ to Ra¹⁰³ each independently represent a hydrogen atom, a monovalent chained saturated hydrocarbon group having 1 to 10 carbon atoms, or a monovalent aliphatic cyclic saturated hydrocarbon group having 3 to 20 carbon atoms. Part or all of hydrogen atoms of the chained saturated hydrocarbon group and the aliphatic cyclic saturated hydrocarbon group may be replaced. Two or more of Ra¹⁰¹ to Ra¹⁰³ may be bonded to each other to form a cyclic structure. In the formula (a1-r2-3), Yaa represents a carbon atom. Xaa is a group that forms an aliphatic cyclic group together with Yaa. Ra¹⁰⁴ represents an aromatic hydrocarbon group that may include a substituent. In the formula (a1-r2-4), Ra′¹² and Ra′¹³ each independently represent a monovalent chain saturated hydrocarbon group having 1 to 10 carbon atoms. Part or all of hydrogen atoms in the chain saturated hydrocarbon group may be replaced. Ra′¹⁴ is a hydrocarbon group which may have a substituent. * indicates a bond.

In the formula (a1-r2-1), Ra⁰³¹ is preferably a chained alkyl group, and is preferably a linear or branched alkyl group having 1 to 12 carbon atoms which may be partially replaced by a halogen atom or a heteroatom-containing group.

The linear alkyl group in Ra⁰³¹ has 1 to 12 carbon atoms, preferably has 1 to 10 carbon atoms, and particularly preferably has 1 to 5 carbon atoms.

Examples of the branched alkyl group in Ra⁰³¹ include groups same as those described above for Ra′⁴.

The alkyl group in Ra⁰³¹ may be partially replaced by a halogen atom or a heteroatom-containing group. For example, a part of hydrogen atoms constituting the alkyl group may be replaced by a halogen atom or a heteroatom-containing group. A part of carbon atoms (such as methylene groups) constituting the alkyl group may be replaced by a heteroatom-containing group.

Examples of the heteroatom used here include an oxygen atom, a sulfur atom, and a nitrogen atom. Examples of the heteroatom-containing group include (—O—), —C(═O)—O—, —O—C(═O)—, —C(═O)—, —O—C(═O)—O—, —C(═O)—NH—, —NH—, —S—, —S(═O)₂—, and —S(═O)₂—O—.

In the formula (a1-r2-1), Xab⁰ (a group which forms an alicyclic hydrocarbon group together with Yab⁰) is preferably a group described as the aliphatic hydrocarbon group (the alicyclic hydrocarbon group) which is a monocyclic group or polycyclic group for Ra′³ in the above formula (a1-r-1). Among them, an alicyclic hydrocarbon group is preferred, a monocyclic alicyclic hydrocarbon group is more preferred, and a group in which two or more hydrogen atoms are removed from a monocycloalkane is still more preferred. The monocycloalkane preferably has 3 to 8 carbon atoms, and specific examples thereof include cyclopentane, cyclohexane, cycloheptane, and cyclooctane.

In the formula (a1-r2-2), examples of the cyclic hydrocarbon group formed by Xa together with Ya include a group in which one or more hydrogen atoms are further removed from the cyclic monovalent hydrocarbon group (the aliphatic hydrocarbon group) in Ra′⁴ in the above formula (a1-r-2).

The cyclic hydrocarbon group formed by Xa together with Ya may include a substituent. Examples of the substituent include those same as the substituent that the cyclic hydrocarbon group in Ra′⁴ may have.

Examples of the monovalent chained saturated hydrocarbon group having 1 to 10 carbon atoms in Ra¹⁰¹ to Ra¹⁰³ in the formula (a1-r2-2) include a methyl group, an ethyl group, a propyl group, a butyl group, a pentyl group, a hexyl group, a heptyl group, an octyl group, and a decyl group.

Examples of the monovalent aliphatic cyclic saturated hydrocarbon group having 3 to 20 carbon atoms in Ra¹⁰¹ to Ra¹⁰³ include monocyclic aliphatic saturated hydrocarbon groups such as a cyclopropyl group, a cyclobutyl group, a cyclopentyl group, a cyclohexyl group, a cycloheptyl group, a cyclooctyl group, a cyclodecyl group, and a cyclododecyl group; and polycyclic aliphatic saturated hydrocarbon groups such as a bicyclo[2.2.2]octanyl group, a tricyclo[5.2.1.02,6]decanyl group, a tricyclo[3.3.1.13,7]decanyl group, a tetracyclo[6.2.1.13,6.02,7]dodecanyl group, and an adamantyl group.

Among them, from the viewpoint of ease of synthesis, Ra¹⁰¹ to Ra¹⁰³ are preferably a hydrogen atom or a monovalent chained saturated hydrocarbon group having 1 to 10 carbon atoms, more preferably a hydrogen atom, a methyl group, or an ethyl group, and particularly preferably a hydrogen atom.

Examples of the substituent that the chain saturated hydrocarbon group or the aliphatic cyclic saturated hydrocarbon group represented by any one of Ra¹⁰¹ to Ra¹⁰³ may include groups same as the above Ra⁰⁵.

Examples of the group containing a carbon-carbon double bond generated by two or more of Ra¹⁰¹ to Ra¹⁰³ bonding to each other to form a cyclic structure include a cyclopentenyl group, a cyclohexenyl group, a methylcyclopentenyl group, a methylcyclohexenyl group, a cyclopentylideneethenyl group, and a cyclohexylideneethenyl group. Among them, from the viewpoint of ease of synthesis, a cyclopentenyl group, a cyclohexenyl group, and a cyclopentylideneethenyl group are preferred.

In the formula (a1-r2-3), the aliphatic cyclic group formed by Xaa together with Yaa is preferably a group described as the aliphatic hydrocarbon group which is a monocyclic group or a polycyclic group for Ra′⁴ in the formula (a1-r-2).

Examples of the aromatic hydrocarbon group in Ra¹⁰⁴ in the formula (a1-r2-3) include a group in which one or more hydrogen atoms are removed from an aromatic hydrocarbon ring having 5 to 30 carbon atoms. Among them, Ra¹⁰⁴ is preferably a group in which one or more hydrogen atoms are removed from an aromatic hydrocarbon ring having 6 to 15 carbon atoms, more preferably a group in which one or more hydrogen atoms are removed from benzene, naphthalene, anthracene, or phenanthrene, still more preferably a group in which one or more hydrogen atoms are removed from benzene, naphthalene, or anthracene, particularly preferably a group in which one or more hydrogen atoms are removed from benzene or naphthalene, and most preferably a group in which one or more hydrogen atoms are removed from benzene.

Examples of the substituent that Ra¹⁰⁴ in the formula (a1-r2-3) may have include a methyl group, an ethyl group, a propyl group, a hydroxy group, a carboxy group, a halogen atom, an alkoxy group (such as a methoxy group, an ethoxy group, a propoxy group, and a butoxy group), and an alkyloxycarbonyl group.

In the formula (a1-r2-4), Ra′¹² and Ra′¹³ each independently represent a monovalent chained saturated hydrocarbon group having 1 to 10 carbon atoms. Examples of the monovalent chained saturated hydrocarbon group having 1 to 10 carbon atoms in Ra′¹² and Ra′¹³ include those same as the monovalent chained saturated hydrocarbon group having 1 to 10 carbon atoms in Ra¹⁰¹ to Ra¹⁰³. Part or all of hydrogen atoms of the chained saturated hydrocarbon group may be replaced.

Among them, Ra′¹² and Ra′¹³ are more preferably an alkyl group having 1 to 5 carbon atoms, still more preferably a methyl group or an ethyl group, and particularly preferably a methyl group.

When the chained saturated hydrocarbon groups represented by Ra′¹² and Ra′¹³ are replaced, examples of the substituent include groups same as those described above Ra⁰⁵.

In the formula (a1-r2-4), Ra′¹⁴ represents a hydrocarbon group that may include a substituent. Examples of the hydrocarbon group in Ra′¹⁴ include a linear or branched alkyl group and a cyclic hydrocarbon group.

The linear alkyl group in Ra′¹⁴ preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferred, and a methyl group or an ethyl group is more preferred.

The branched alkyl group in Ra′¹⁴ preferably has 3 to 10 carbon atoms, and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. An isopropyl group is preferred.

When Ra′¹⁴ is a cyclic hydrocarbon group, the hydrocarbon group may be an aliphatic hydrocarbon group or an aromatic hydrocarbon group, and may be a polycyclic group or a monocyclic group.

The aliphatic hydrocarbon group which is a monocyclic group is preferably a group in which one hydrogen atom is removed from a monocycloalkane. The monocycloalkane preferably has 3 to 6 carbon atoms, and specific examples thereof include cyclopentane and cyclohexane.

The aliphatic hydrocarbon group which is a polycyclic group is preferably a group in which one hydrogen atom is removed from a polycycloalkane. The polycycloalkane is preferably a group having 7 to 12 carbon atoms, and specific examples thereof include adamantane, norbornane, isobornane, tricyclo[5.2.1.02,6]decane, and tetracyclododecane.

Examples of the aromatic hydrocarbon group in Ra′¹⁴ include those same as the aromatic hydrocarbon group in Ra¹⁰⁴. Among them, Ra′¹⁴ is preferably a group in which one or more hydrogen atoms are removed from an aromatic hydrocarbon ring having 6 to 15 carbon atoms, more preferably a group in which one or more hydrogen atoms are removed from benzene, naphthalene, anthracene, or phenanthrene, still more preferably a group in which one or more hydrogen atoms are removed from benzene, naphthalene, or anthracene, particularly preferably a group in which one or more hydrogen atoms are removed from naphthalene or anthracene, and most preferably a group in which one or more hydrogen atoms are removed from naphthalene.

Examples of the substituent that Ra′¹⁴ may have include those same as the substituents that Ra¹⁰⁴ may have.

When Ra′¹⁴ in the formula (a1-r2-4) is a naphthyl group, a position at which Ra′¹⁴ bonds to a tertiary carbon atom in the formula (a1-r2-4) may be either 1-position or 2-position of the naphthyl group.

When Ra′¹⁴ in the formula (a1-r2-4) is an anthryl group, a position at which Ra′¹⁴ bonds to a tertiary carbon atom in the formula (a1-r2-4) may be any of 1-position, 2-position, and 9-position of the anthryl group.

Specific examples of the group represented by the formula (a1-r2-1) are shown below.

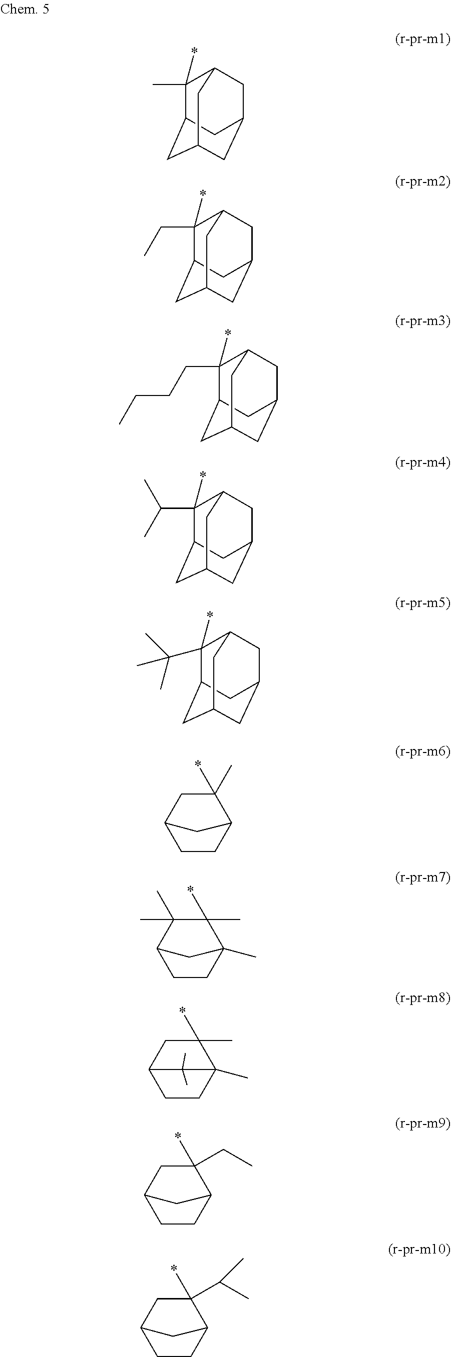

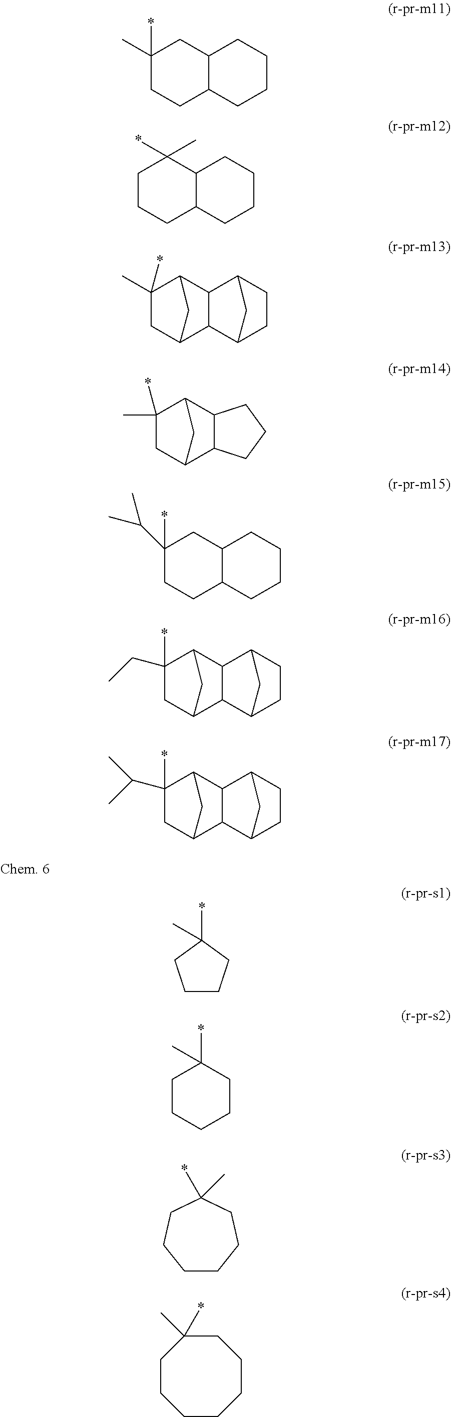

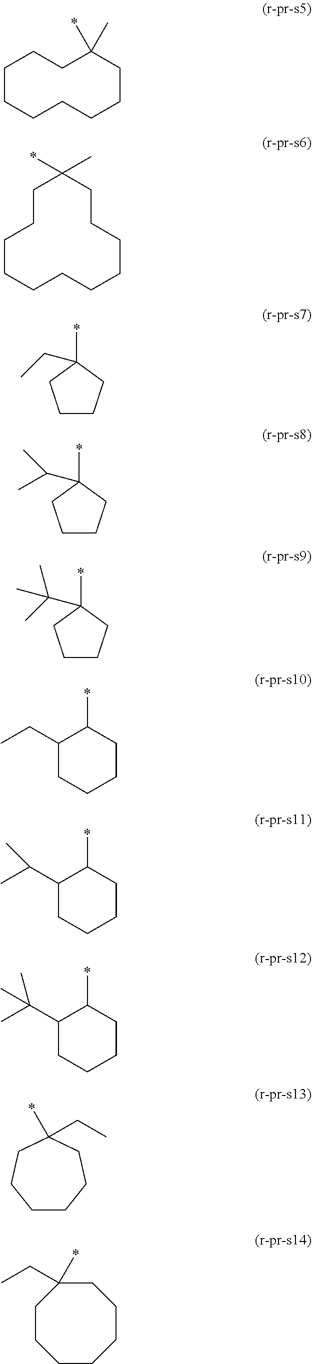

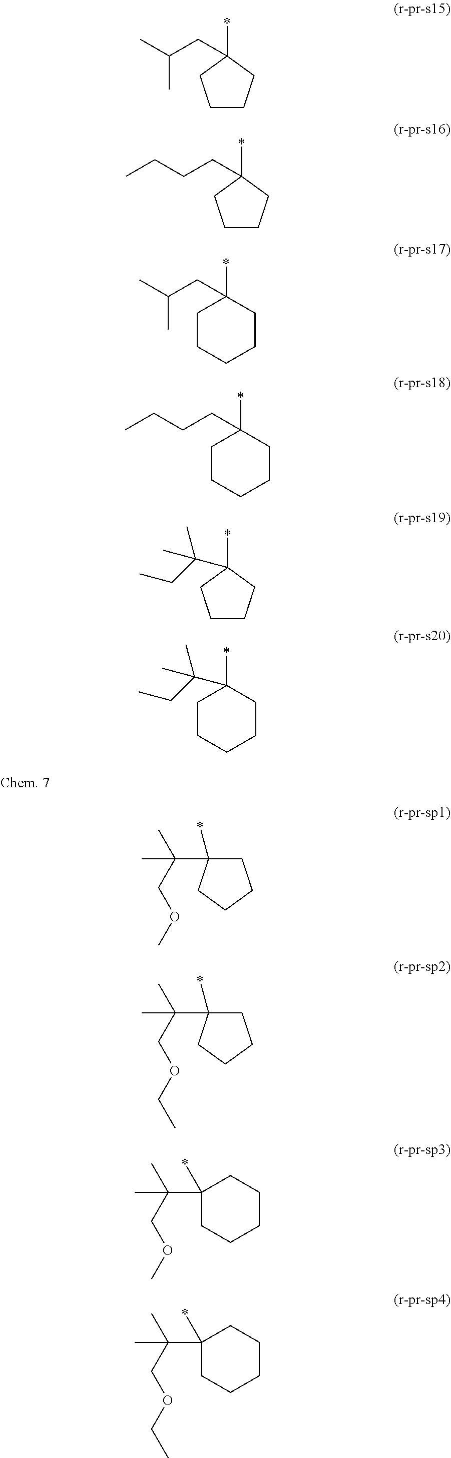

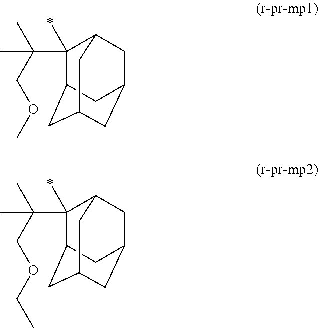

Specific examples of the group represented by the formula (a1-r2-2) are shown below.

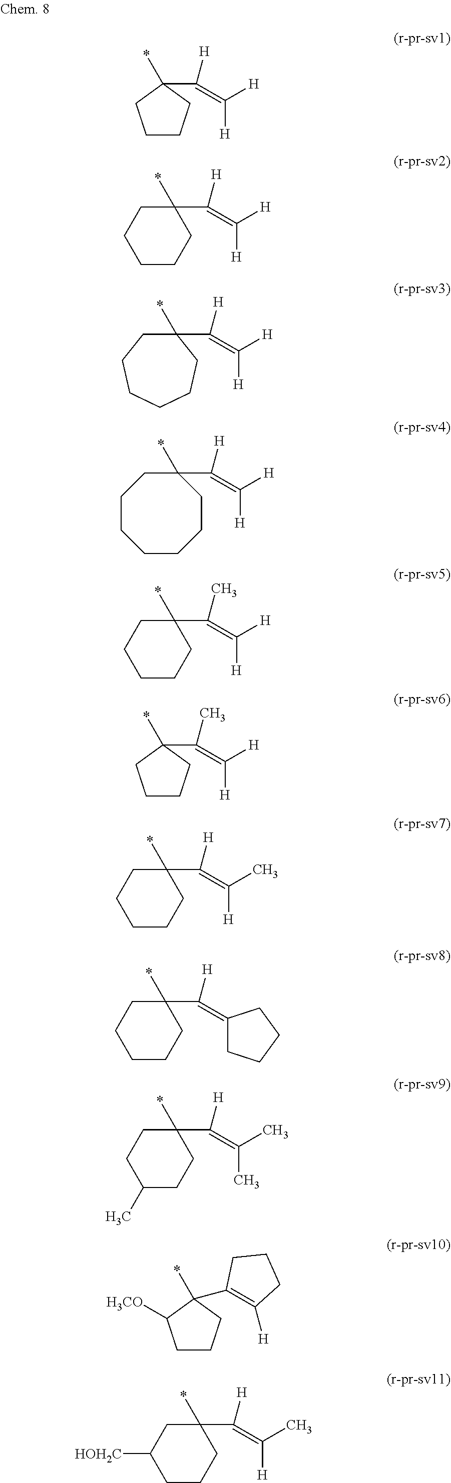

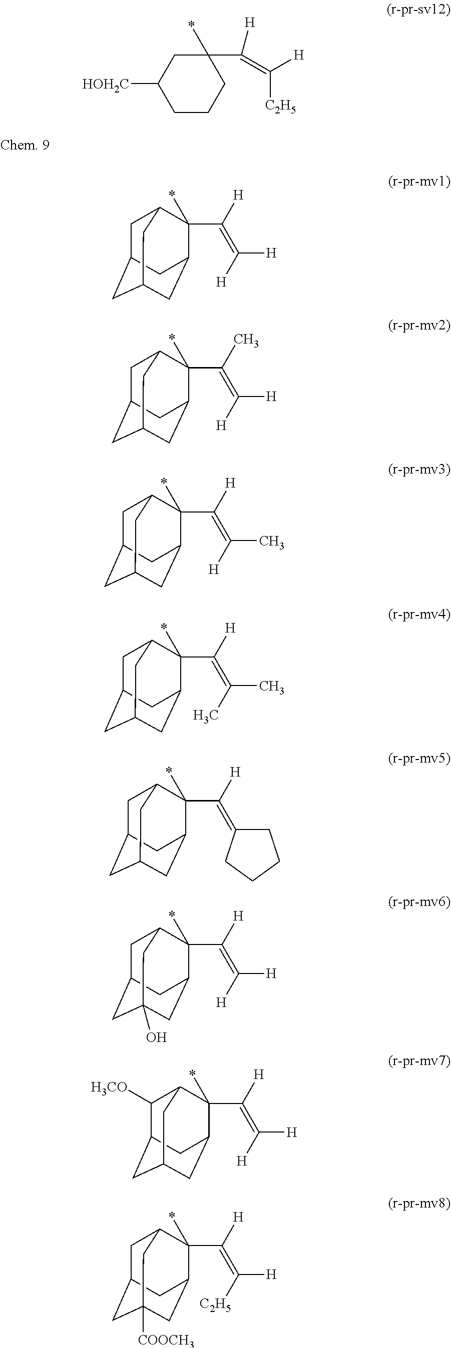

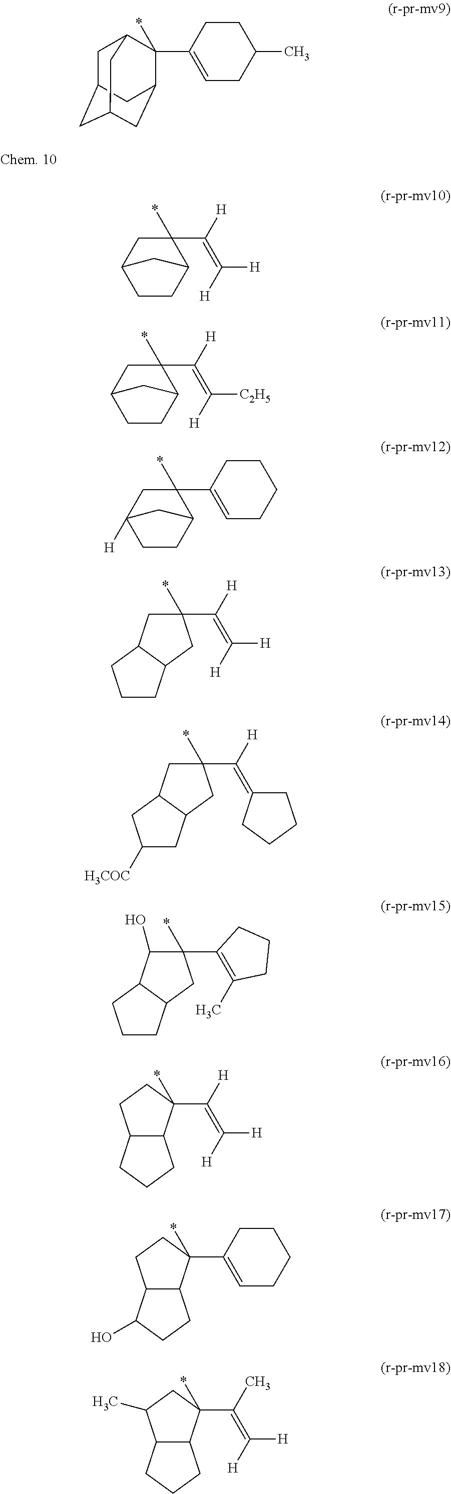

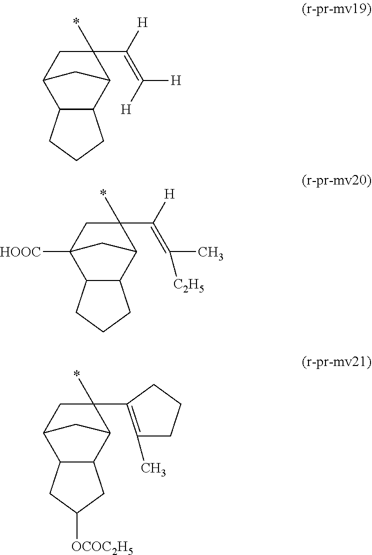

Specific examples of the group represented by the formula (a1-r2-3) are shown below.

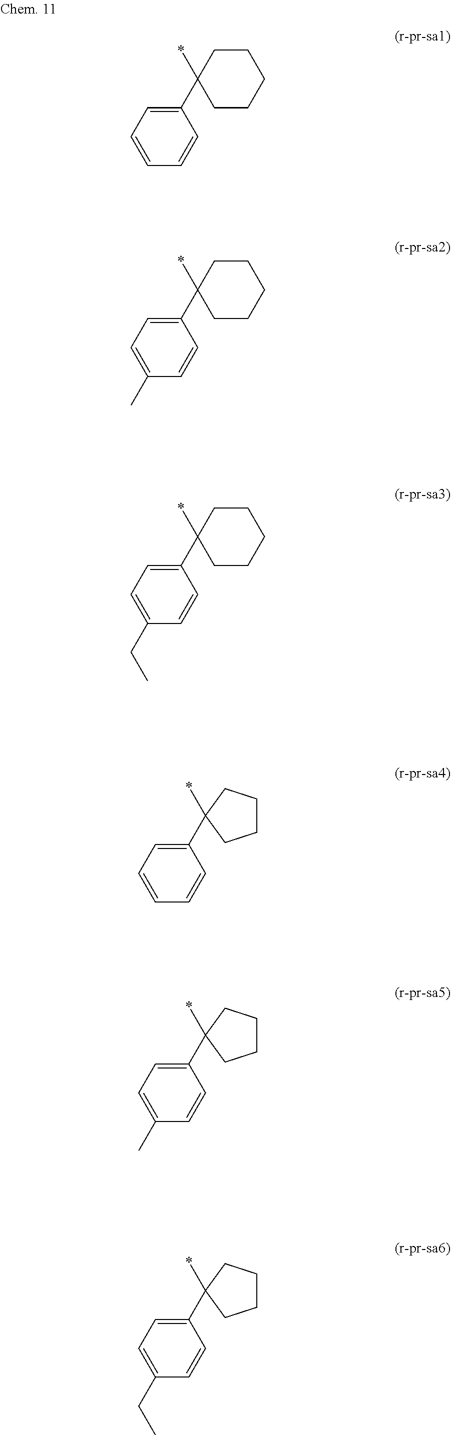 [SMILES: *C1(c2ccccc2)CCCCC1 ; *C1(c2ccc(C)cc2)CCCCC1 ; *C1(c2ccc(CC)cc2)CCCCC1 ; *C1(c2ccccc2)CCCC1 ; *C1(c2ccc(C)cc2)CCCC1 ; *C1(c2ccc(CC)cc2)CCCC1]

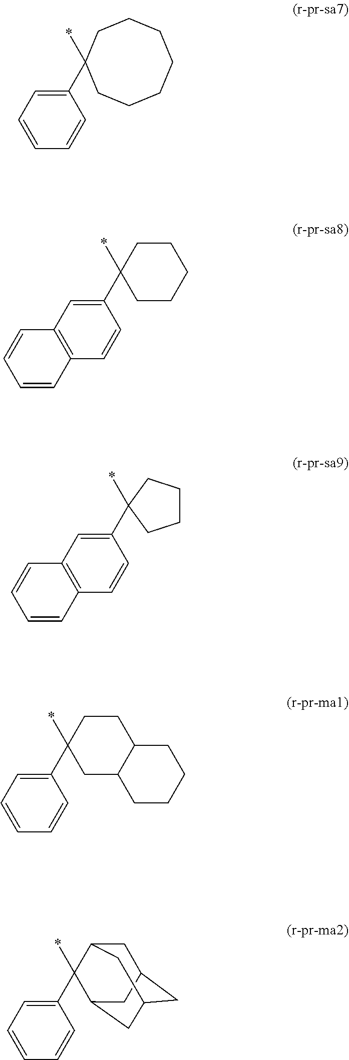 [SMILES: *C1(c2ccccc2)CCCCCCC1 ; *C1(c2ccc3ccccc3c2)CCCCC1 ; *C1(c2ccc3ccccc3c2)CCCC1 ; *C1(c2ccccc2)CCC2CCCCC2C1 ; *C1(c2ccccc2)C2CC3CC(C2)CC1C3]

Specific examples of the group represented by the formula (a1-r2-4) are shown below.

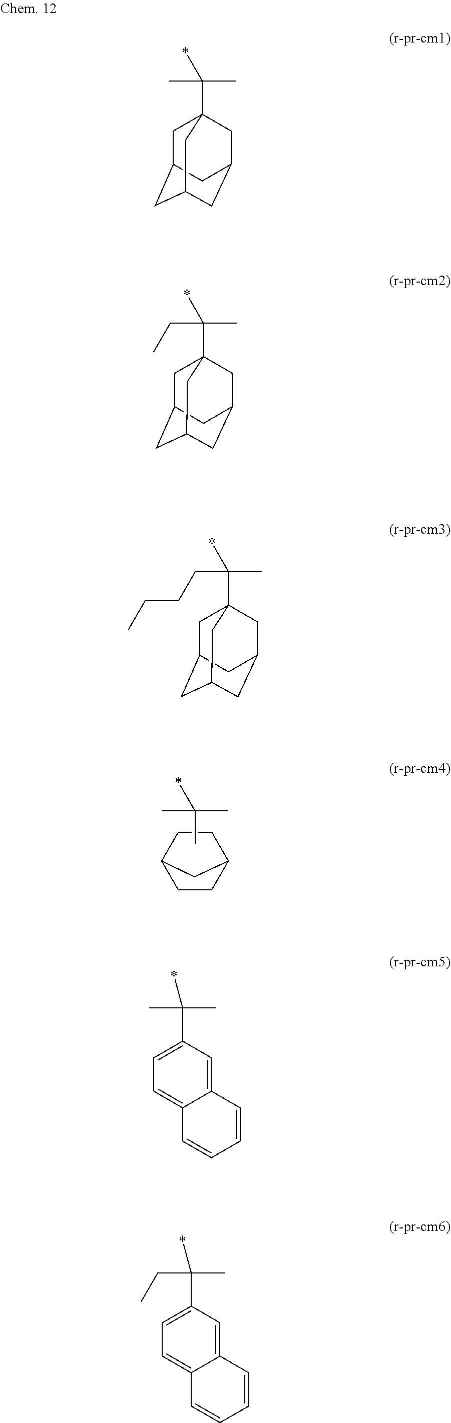

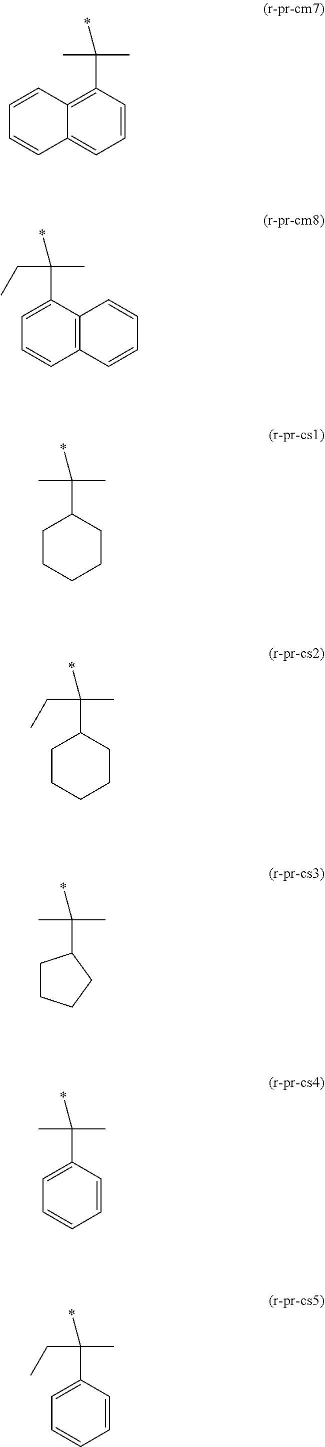

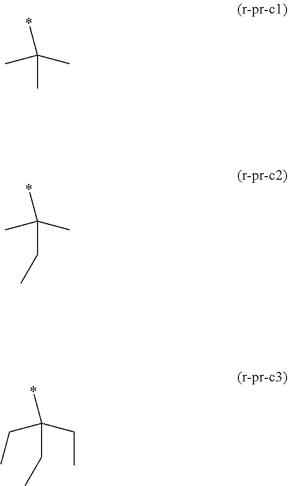

### Tertiary Alkyloxycarbonyl Acid-Dissociable Group:

Among the polar groups, examples of the acid-dissociable group that protects a hydroxy group include an acid-dissociable group represented by the following formula (a1-r-3) (hereinafter, for convenience, may be referred to as “tertiary alkyloxycarbonyl acid-dissociable group”).

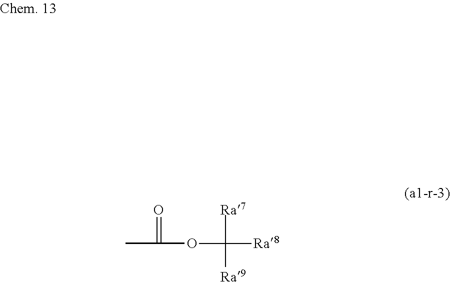

In the formula, Ra′⁷ to Ra′⁹ each represent an alkyl group.

In the formula (a1-r-3), Ra′⁷ to Ra′⁹ each preferably represent an alkyl group having 1 to 5 carbon atoms, and more preferably represent 1 to 3 carbon atoms.

The total number of carbon atoms in each alkyl group is preferably 3 to 7, more preferably 3 to 5, and most preferably 3 or 4.

As the acid-dissociable group, among the groups represented by the general formulae (a1-r2-1) to (a1-r2-4), a group represented by the general formula (a1-r2-1) or (a1-r2-4) is preferred.

Specific examples of the structural unit (a1) include a structural unit represented by the following general formula (a1-1).

Structural Unit (a1) Represented by General Formula (a1-1)

In the resist composition according to the embodiment of the present invention, the base material component (A) preferably includes the macromolecular compound (A1) having the structural unit (a1) represented by the following general formula (a1-1).

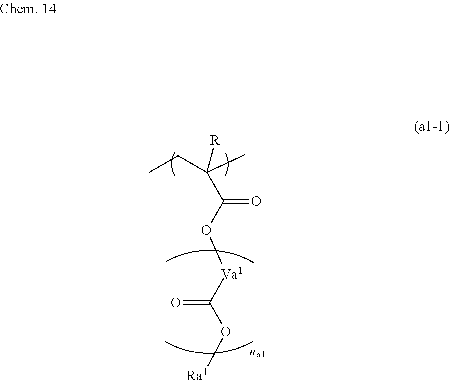

In the formula, R represents a hydrogen atom, an alkyl group having 1 to 5 carbon atoms, or a halogenated alkyl group having 1 to 5 carbon atoms. Va¹ represents a divalent hydrocarbon group which may have an ether bond. nₐ₁ is an integer of 0 to 2. Ra¹ represents an acid-dissociable group.

In the formula (a1-1), the alkyl group having 1 to 5 carbon atoms for R is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. The halogenated alkyl group having 1 to 5 carbon atoms is a group in which part or all of hydrogen atoms of the alkyl group having 1 to 5 carbon atoms are replaced by halogen atoms. Examples of the halogen atom include a fluorine atom, a chlorine atom, a bromine atom, and an iodine atom. A fluorine atom is particularly preferred.

R is preferably a hydrogen atom, an alkyl group having 1 to 5 carbon atoms, or a fluorinated alkyl group having 1 to 5 carbon atoms, and from the viewpoint of industrial availability, a hydrogen atom or a methyl group is most preferred.

In the above formula (a1-1), the divalent hydrocarbon group in Va¹ may be either an aliphatic hydrocarbon group or an aromatic hydrocarbon group.

The aliphatic hydrocarbon group as the divalent hydrocarbon group in Va¹ may be saturated or unsaturated, and generally, is preferably saturated.

More specific examples of the aliphatic hydrocarbon group include a linear or branched aliphatic hydrocarbon group, and an aliphatic hydrocarbon group containing a ring in the structure thereof.

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkyl alkylene groups such as alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkyl ethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyl trimethylene groups such as —CH(CH₃)CH₂CH₂— and —CH₂CH(CH₃)CH₂—; and alkyl tetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

Examples of the aliphatic hydrocarbon group containing a ring in the structure thereof include an alicyclic hydrocarbon group (a group in which two hydrogen atoms are removed from an aliphatic hydrocarbon ring), a group in which an alicyclic hydrocarbon group is bonded to the end of a linear or branched aliphatic hydrocarbon group, and a group in which an alicyclic hydrocarbon group is interposed in the middle of a linear or branched aliphatic hydrocarbon group. Examples of the linear or branched aliphatic hydrocarbon group include those same as the linear aliphatic hydrocarbon group or the branched aliphatic hydrocarbon group.

The alicyclic hydrocarbon group preferably has 3 to 20 carbon atoms, and more preferably has 3 to 12 carbon atoms.

The alicyclic hydrocarbon group may be polycyclic or monocyclic. The monocyclic alicyclic hydrocarbon group is preferably a group in which two hydrogen atoms are removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms are removed from a polycycloalkane. The polycycloalkane is preferably a group having 7 to 12 carbon atoms. Specific examples thereof include adamantane, norbornane, isobornane, tricyclo[5.2.1.02,6]decane, and tetracyclododecane.

The aromatic hydrocarbon group as the divalent hydrocarbon group in Va¹ is a hydrocarbon group having an aromatic ring.

The aromatic hydrocarbon group preferably has 3 to 30 carbon atoms, more preferably has 5 to 30 carbon atoms, still more preferably has 5 to 20 carbon atoms, particularly preferably has 6 to 15 carbon atoms, and most preferably has 6 to 12 carbon atoms. The number of the carbon atoms does not include the number of the carbon atoms in the substituent.

Specific examples of the aromatic ring contained in the aromatic hydrocarbon group include an aromatic hydrocarbon ring such as benzene, biphenyl, fluorene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom.

Specific examples of the aromatic hydrocarbon group include a group in which two hydrogen atoms are removed from the aromatic hydrocarbon ring (an arylene group); a group in which one hydrogen atom of a group in which one hydrogen atom is removed from the aromatic hydrocarbon ring (an aryl group) is replaced by an alkylene group (for example, a group in which one hydrogen atom is further removed from the aryl group in an arylalkyl group such as a benzyl group, a phenethyl group, a 1-naphthylmethyl group, a 2-naphthylmethyl group, a 1-naphthylethyl group, or a 2-naphthylethyl group). The number of carbon atoms of the alkylene group (an alkyl chain in the arylalkyl group) is preferably 1 to 4, more preferably 1 to 2, and particularly preferably 1.

In the formula (a1-1), Ra¹ represents an acid-dissociable group. Examples of the acid-dissociable group include those described above. The acid-dissociable group is preferably an acid-dissociable group represented by each of the above formulae (a1-r2-1) to (a1-r2-4), and more preferably an acid-dissociable group represented by the above formula (a1-r2-1) or (a1-r2-4).

In the above formula (a1-1), nₐ₁ is an integer of 0 to 2. nₐ₁ is preferably 0 or 1, and more preferably 0.

The formula (a1-1) is preferably the following formula (a1-2).

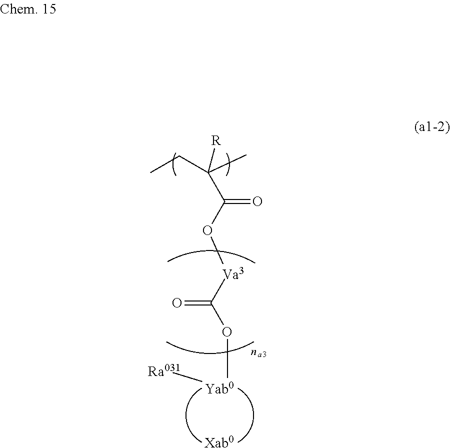

In the general formula (a1-2), R represents a hydrogen atom, an alkyl group having 1 to 5 carbon atoms, or a halogenated alkyl group having 1 to 5 carbon atoms. Va³ represents a divalent linking group. na₃ represents an integer of 0 to 2. Ra⁰³¹ represents an alkyl group, Yab⁰ represents a carbon atom. Xab⁰ represents a group which forms an alicyclic hydrocarbon group together with Yab⁰, and part or all of hydrogen atoms of the alicyclic hydrocarbon group may be replaced.

In the general formula (a1-2), R and Va³ are the same as R and Va¹ in the formula (a1-1), respectively.

In the general formula (a1-2), na₃ is an integer of 0 to 2, preferably 0 or 1, and more preferably 0.

In the general formula (a1-2), Ra⁰³¹, Xab⁰, and Yab⁰ are the same as Ra⁰³¹, Xab⁰, and Yab⁰ in the formula (a1-r2-1), respectively.

In the general formula (a1-2), Ra⁰³¹ is preferably a chained alkyl group among them, and is preferably a monovalent chained alkyl group having 1 to 3 carbon atoms. Specifically, a methyl group, an ethyl group, a propyl group, or an iso-propyl group is more preferred.

Specific examples of the structural unit (a1) are shown below.

In the following formulae, R^{α} represents a hydrogen atom, a methyl group, or a trifluoromethyl group.

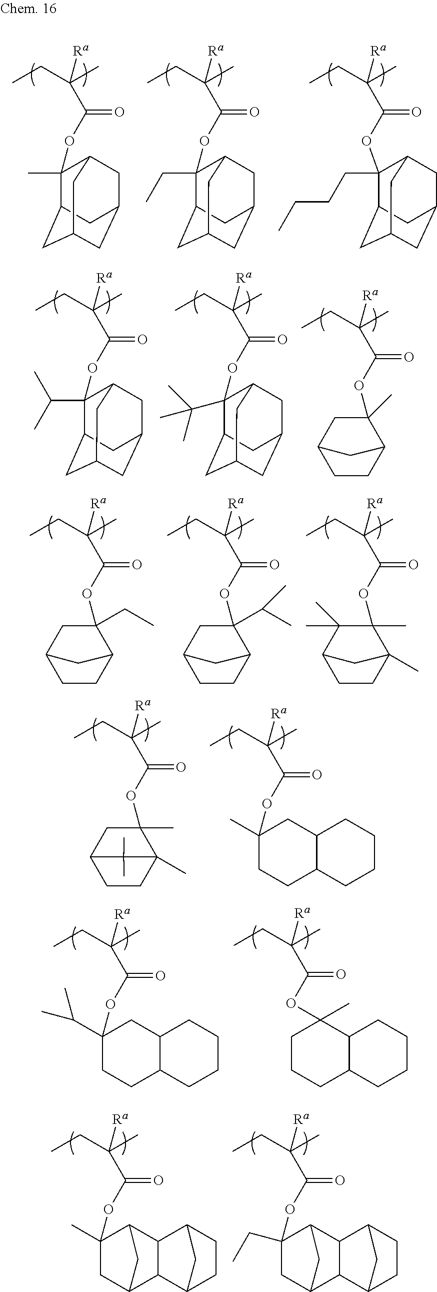

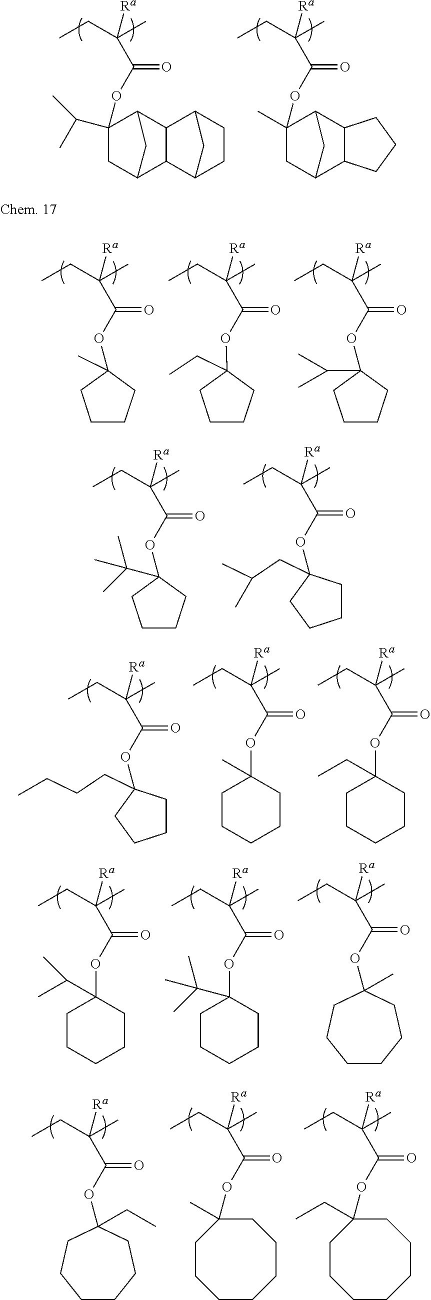

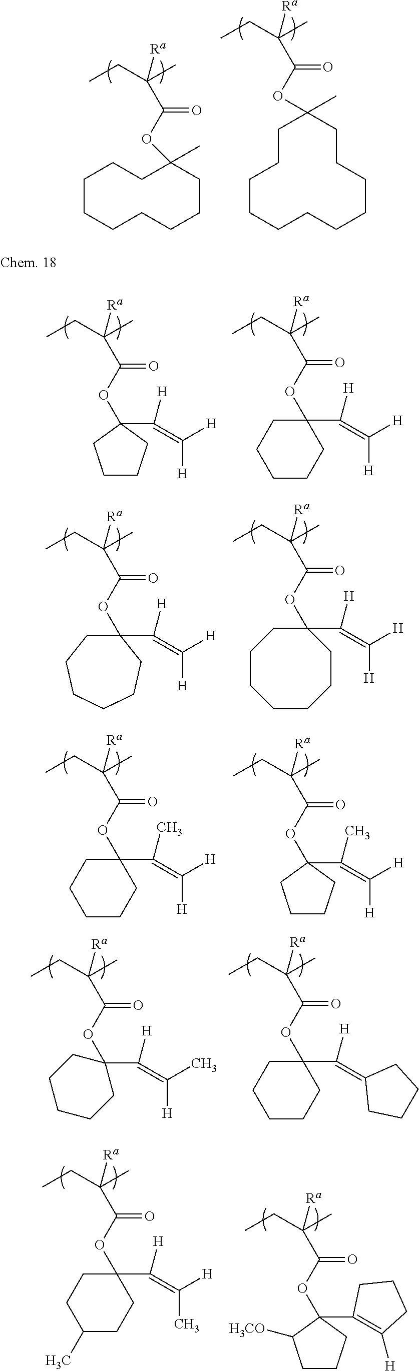

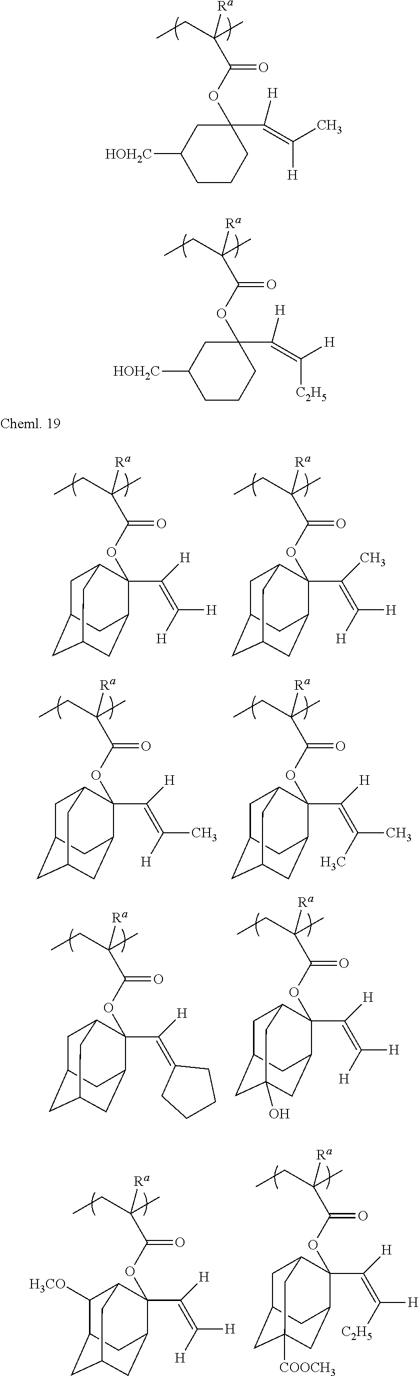

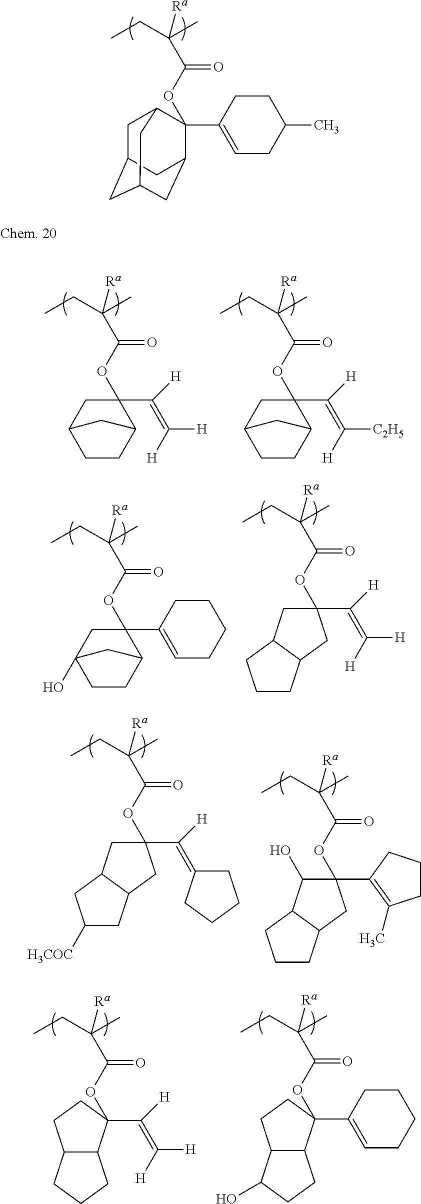

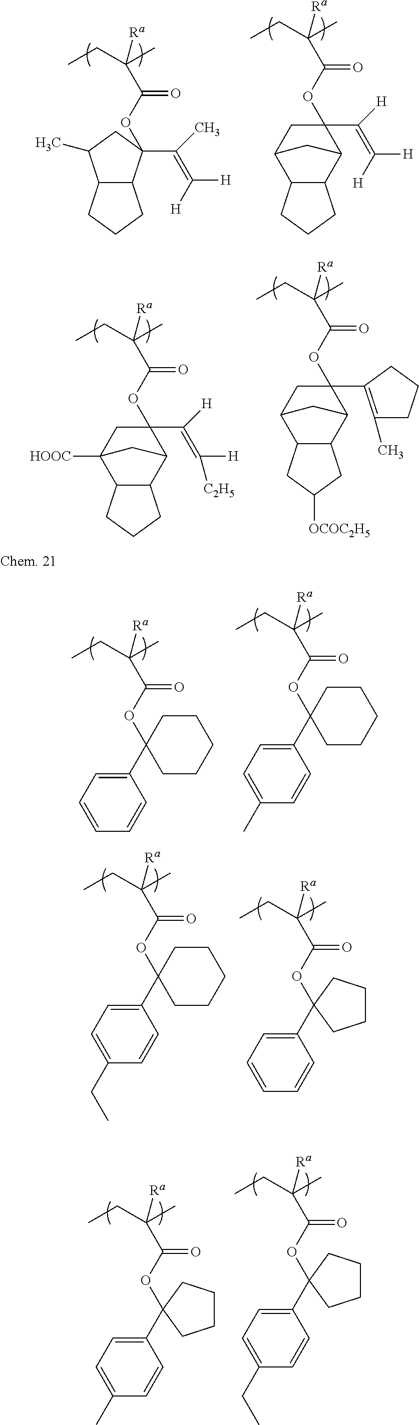

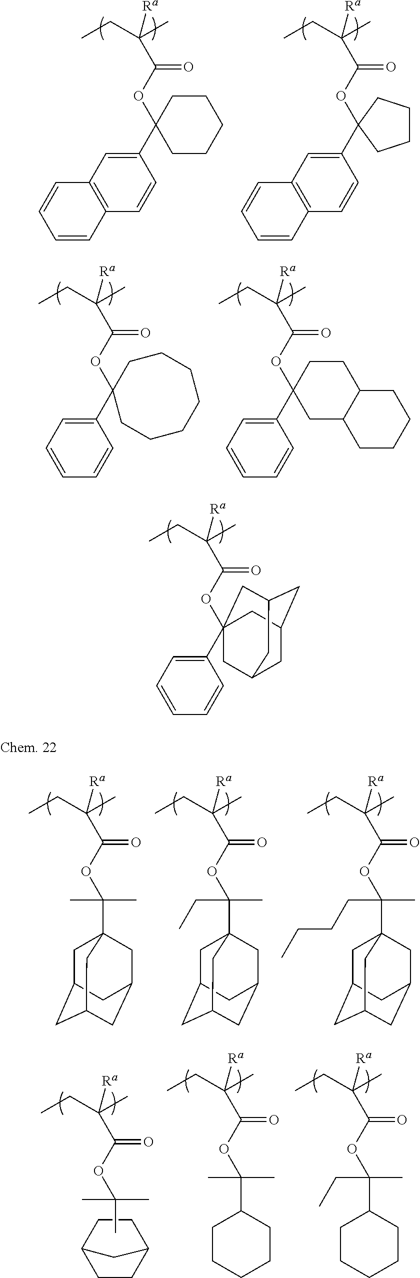

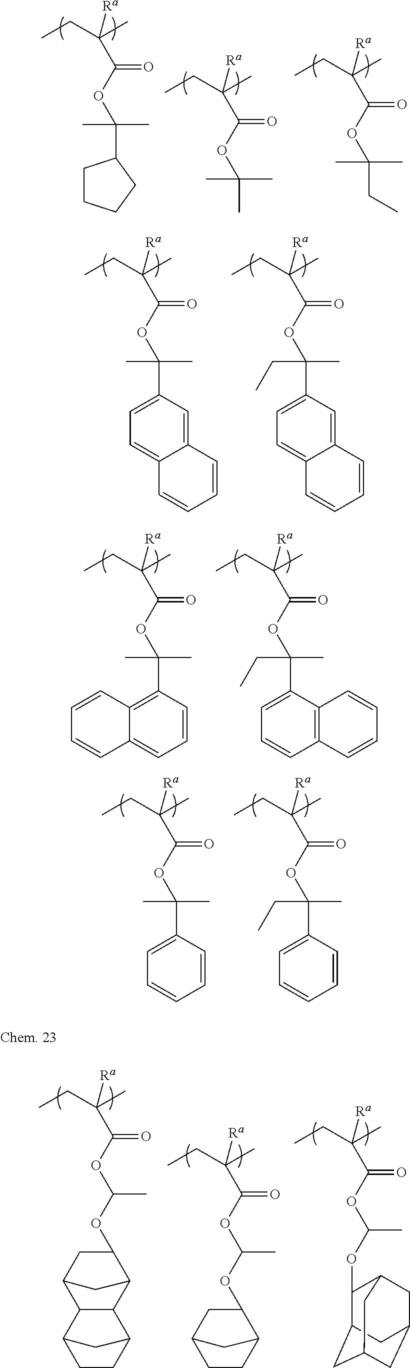

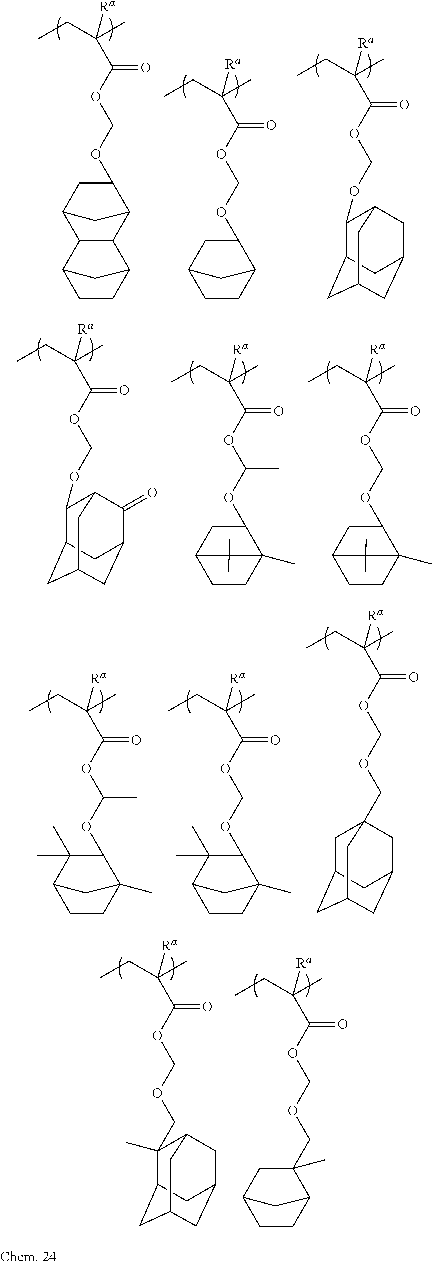

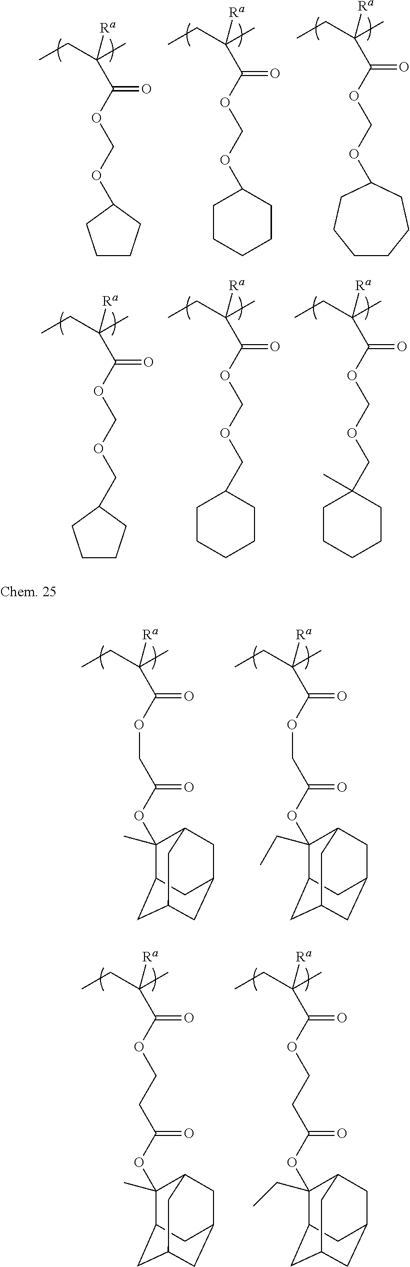

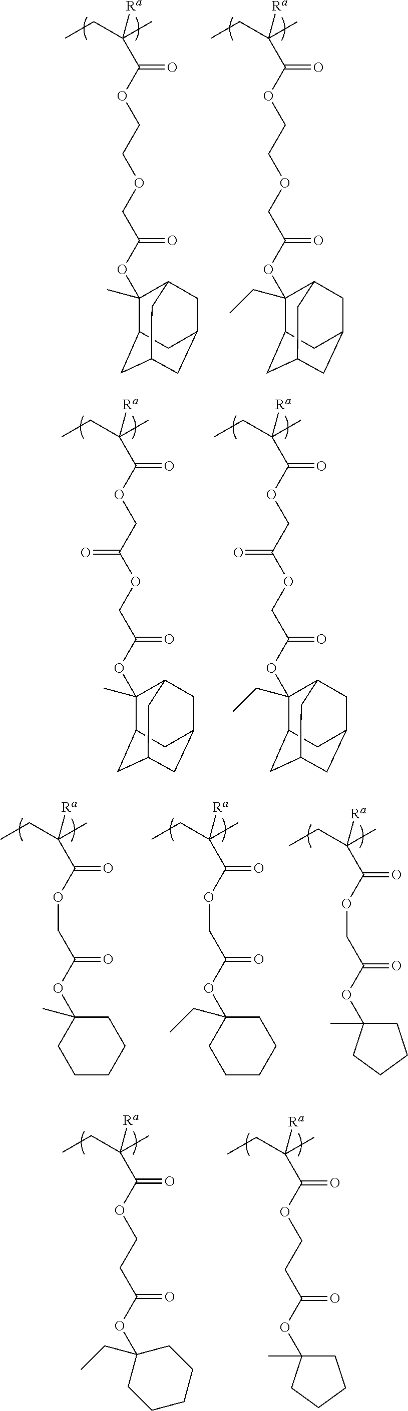

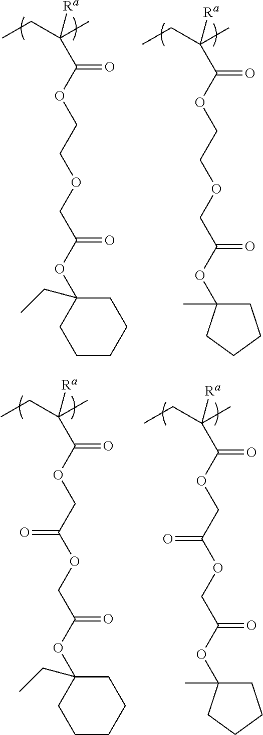

The structural unit (a1) that the component (A1) may have may be one kind or two or more kinds thereof.

In the component (A1), a proportion of the structural unit (a1) is preferably 20 mol % to 80 mol %, more preferably 30 mol % to 70 mol %, and still more preferably 40 mol % to 60 mol %, with respect to the total (100 mol %) of all structural units constituting the component (A1).

When the proportion of the structural unit (a1) is equal to or larger than a lower limit value of the preferred range, lithography properties such as a high sensitivity, a high resolution, and roughness improvement are improved. When the proportion is equal to or less than an upper limit value of the preferred range, a good balance with other structural units can be achieved, and various lithography properties are improved.

In the resist composition according to the embodiment of the present invention, the structural unit (a1) may not contain an acetal acid-dissociable group.

### Structural Unit (a2)

The component (A1) may further include a structural unit (a2) (excluding those corresponding to the structural unit (a1)) including a lactone-containing cyclic group, a —SO₂-containing cyclic group, or a carbonate-containing cyclic group.

The lactone-containing cyclic group, the —SO₂-containing cyclic group, or the carbonate-containing cyclic group of the structural unit (a2) is effective in enhancing adhesion of the resist film to the substrate when the component (A1) is used for forming a resist film. When the structural unit (a2) is contained, for example, an acid diffusion length can be appropriately adjusted, the adhesion of the resist film to the substrate can be enhanced, and the solubility during development can be appropriately adjusted, resulting in improved lithography properties.

The term “lactone-containing cyclic group” refers to a cyclic group containing a ring (a lactone ring) containing —O—C(═O)— in the ring skeleton thereof. When a lactone ring is counted as a first ring, a group having only a lactone ring is referred to as a monocyclic group, and a group further having another ring structure is referred to as a polycyclic group regardless of the structure. The lactone-containing cyclic group may be a monocyclic group or a polycyclic group.

The lactone-containing cyclic group in the structural unit (a2) is not particularly limited, and any group can be used. Specific examples thereof include a group represented by any one of the following general formulae (a2-r-1) to (a2-r-7).

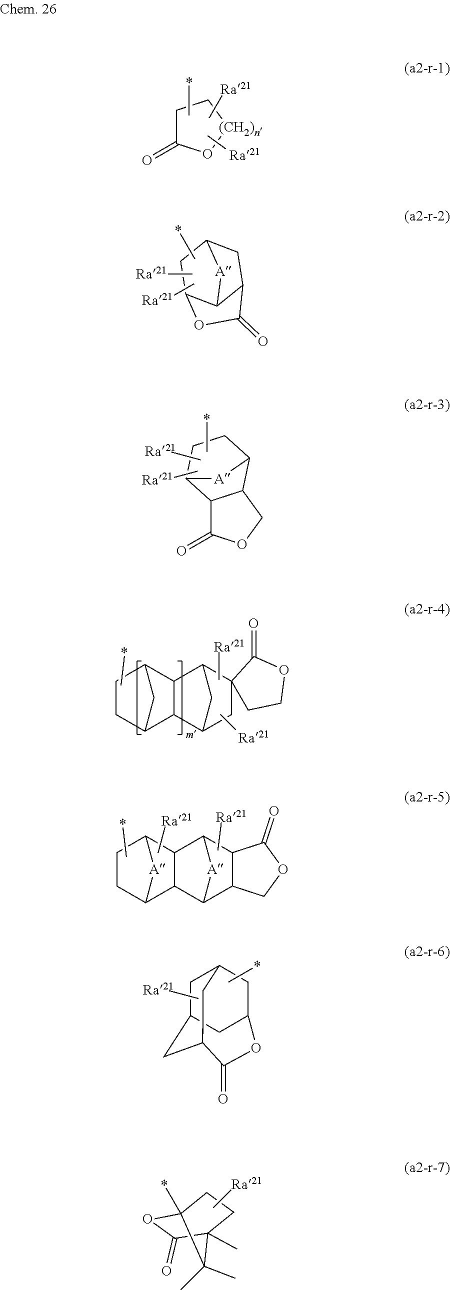

In the general formulae (a2-r-1) to (a2-r-7), Ra′²¹'s each independently represent a hydrogen atom, an alkyl group, an alkoxy group, a halogen atom, a halogenated alkyl group, a hydroxy group, —COOR″, —OC(═O)R″, a hydroxyalkyl group, or a cyano group. R″ represents a hydrogen atom, an alkyl group, a lactone-containing cyclic group, a carbonate-containing cyclic group, or a —SO₂-containing cyclic group. A″ represents an alkylene group having 1 to 5 carbon atoms that may contain an oxygen atom (—O—) or a sulfur atom (—S—), an oxygen atom, or a sulfur atom; n′ is an integer of 0 to 2, and m′ is 0 or 1. * indicates a bond.

In the general formulae (a2-r-1) to (a2-r-7), the alkyl group in Ra′²¹ is preferably an alkyl group having 1 to 6 carbon atoms. The alkyl group is preferably linear or branched. Specific examples thereof include a methyl group, an ethyl group, a propyl group, an iso-propyl group, an n-butyl group, an iso-butyl group, a tert-butyl group, a pentyl group, an iso-pentyl group, a neopentyl group, and a hexyl group. Among them, a methyl group or an ethyl group is preferred, and a methyl group is particularly preferred.

The alkoxy group in Ra′²¹ is preferably an alkoxy group having 1 to 6 carbon atoms. The alkoxy group is preferably linear or branched. Specific examples thereof include a group in which an oxygen atom (—O—) is linked to an alkyl group described as the alkyl group in Ra′²¹.

Examples of the halogen atom in Ra′²¹ include a fluorine atom, a chlorine atom, a bromine atom, and an iodine atom, and a fluorine atom is preferred.

Examples of the halogenated alkyl group in Ra′²¹ include a group in which a part or all of hydrogen atoms in the alkyl group in Ra′²¹ are replaced by halogen atoms. The halogenated alkyl group is preferably a fluorinated alkyl group, and particularly preferably a perfluoroalkyl group.

In —COOR″ and —OC(═O)R″ in Ra′²¹, R″ represents a hydrogen atom, an alkyl group, a lactone-containing cyclic group, a carbonate-containing cyclic group, or a —SO₂-containing cyclic group.

The alkyl group in R″ may be linear, branched, or cyclic, and preferably has 1 to 15 carbon atoms.

When R″ is a linear or branched alkyl group, R″ preferably has 1 to 10 carbon atoms, and more preferably has 1 to 5 carbon atoms, and is particularly preferably a methyl group or an ethyl group.

When R″ is a cyclic alkyl group, R″ preferably has 3 to 15 carbon atoms, more preferably has 4 to 12 carbon atoms, and most preferably has 5 to 10 carbon atoms. Specific examples thereof include a group in which one or more hydrogen atoms are removed from a monocycloalkane that may or may not be replaced by a fluorine atom or a fluorinated alkyl group; and a group in which one or more hydrogen atoms are removed from a polycycloalkane such as a bicycloalkane, a tricycloalkane, or a tetracycloalkane. More specific examples thereof include a group in which one or more hydrogen atoms are removed from a monocycloalkane such as cyclopentane or cyclohexane; and a group in which one or more hydrogen atoms are removed from a polycycloalkane such as adamantane, norbornane, isobornane, tricyclo[5.2.1.02,6]decane, or tetracyclododecane.

Examples of the lactone-containing cyclic group in R″ include those same as the group represented by any one of the general formulae (a2-r-1) to (a2-r-7).

The carbonate-containing cyclic group in R″ is the same as the carbonate-containing cyclic group described below, and specific examples thereof include a group represented by any one of general formulae (ax3-r-1) to (ax3-r-3).

The —SO₂-containing cyclic group for R″ is the same as the —SO₂— containing cyclic group described below, and specific examples thereof include a group represented by any one of general formulae (a5-r-1) to (a5-r-4).

The hydroxyalkyl group in Ra′²¹ preferably has 1 to 6 carbon atoms, and specific examples thereof include a group in which at least one hydrogen atom in the alkyl group in Ra′²¹ is replaced by a hydroxy group.

In the general formulae (a2-r-2), (a2-r-3), and (a2-r-5), the alkylene group having 1 to 5 carbon atoms in A″ is preferably a linear or branched alkylene group, and examples thereof include a methylene group, an ethylene group, an n-propylene group, and an isopropylene group. When the alkylene group contains an oxygen atom or a sulfur atom, specific examples thereof include a group in which —O— or —S— is interposed at the end of the alkylene group or between carbon atoms of the alkylene group, and examples thereof include —O—CH₂—, —CH₂—O—CH₂—, —S—CH₂—, and —CH₂—S—CH₂—. A″ is preferably an alkylene group having 1 to 5 carbon atoms or —O—, more preferably an alkylene group having 1 to 5 carbon atoms, and most preferably a methylene group.

Specific examples of the group represented by any one of the general formulae (a2-r-1) to (a2-r-7) are shown below.

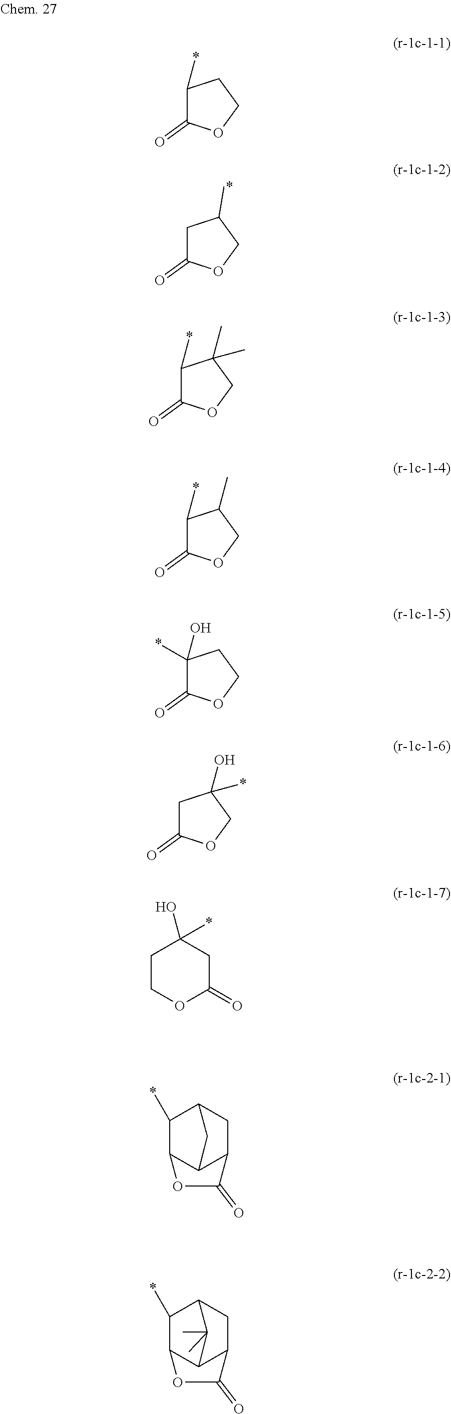

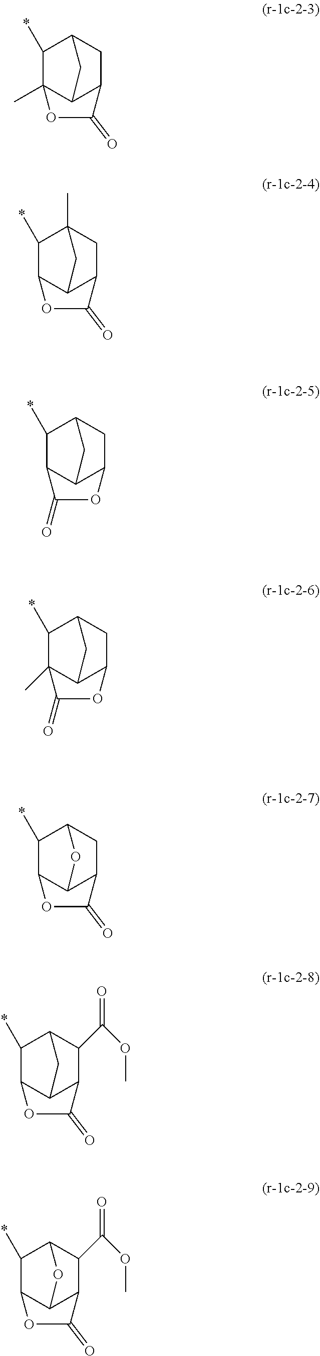

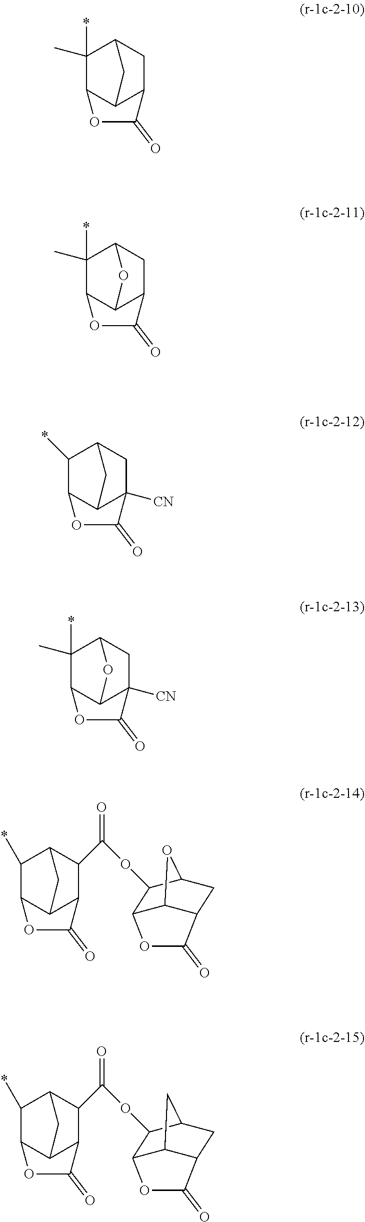

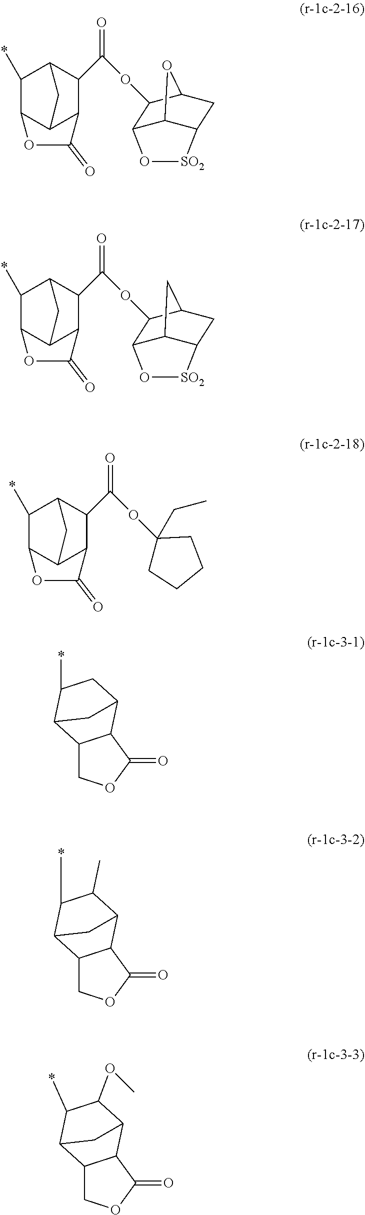

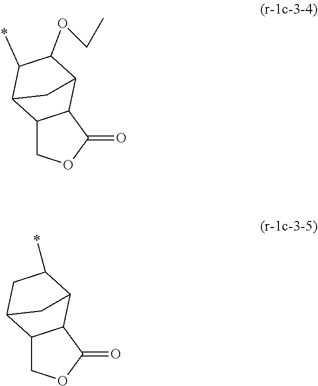

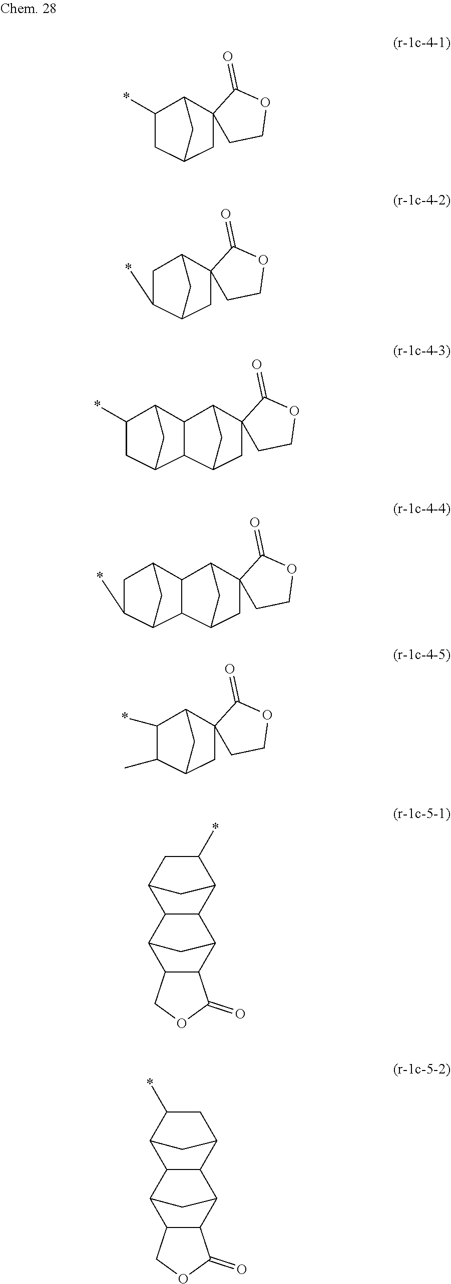

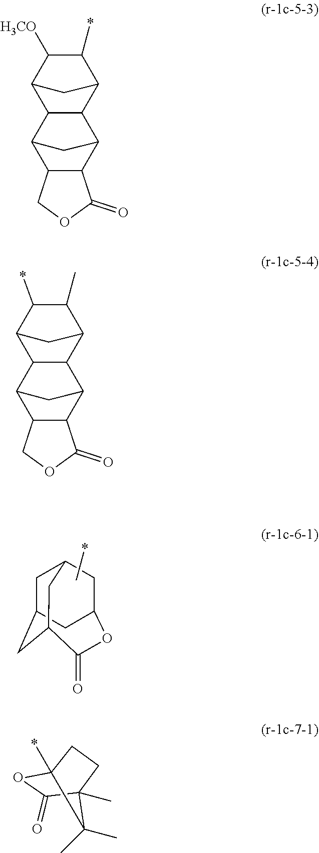

The term “—SO₂-containing cyclic group” refers to a cyclic group containing a ring containing —SO₂— in the ring skeleton thereof, and specifically, the —SO₂-containing cyclic group is a cyclic group in which a sulfur atom(S) in —SO₂-forms a part of the ring skeleton of the cyclic group. When a ring containing —SO₂— in the ring skeleton thereof is counted as a first ring, a group having only the ring is referred to as a monocyclic group, and a group further having another ring structure is referred to as a polycyclic group regardless of the structure. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂-forms a part of the ring skeleton.

Specific examples of the —SO₂-containing cyclic group include a group represented by any one of the following general formulae (a5-r-1) to (a5-r-4).

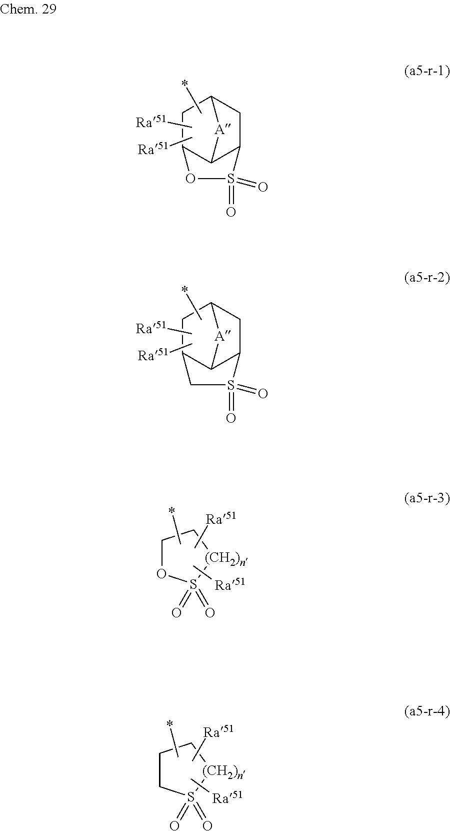

In the general formulae (a5-r-1) to (a5-r-4), Ra′⁵¹'s each independently represent a hydrogen atom, an alkyl group, an alkoxy group, a halogen atom, a halogenated alkyl group, a hydroxy group, —COOR″, —OC(═O)R″, a hydroxyalkyl group, or a cyano group. R″ represents a hydrogen atom, an alkyl group, a lactone-containing cyclic group, a carbonate-containing cyclic group, or a —SO₂-containing cyclic group. A″ represents an alkylene group having 1 to 5 carbon atoms that may contain an oxygen atom or a sulfur atom, an oxygen atom, or a sulfur atom. n′ is an integer of 0 to 2.

In the general formulae (a5-r-1) and (a5-r-2), A″ is the same as A″ in the general formulae (a2-r-2), (a2-r-3) and (a2-r-5).

Examples of the alkyl group, alkoxy group, halogen atom, halogenated alkyl group, —COOR″, —OC(═O)R″, and hydroxyalkyl group in Ra′⁵¹ include those same as those described in the description of Ra′²¹ in the general formulae (a2-r-1) to (a2-r-7).

Specific examples of the group represented by any one of the general formulae (a5-r-1) to (a5-r-4) are shown below. In the formula, “Ac” represents an acetyl group.

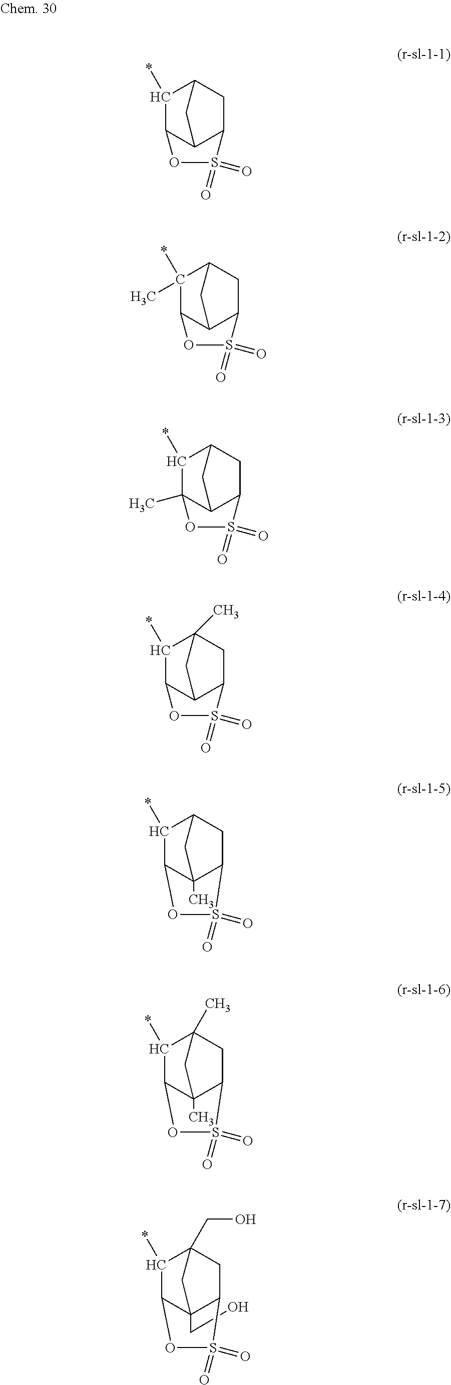

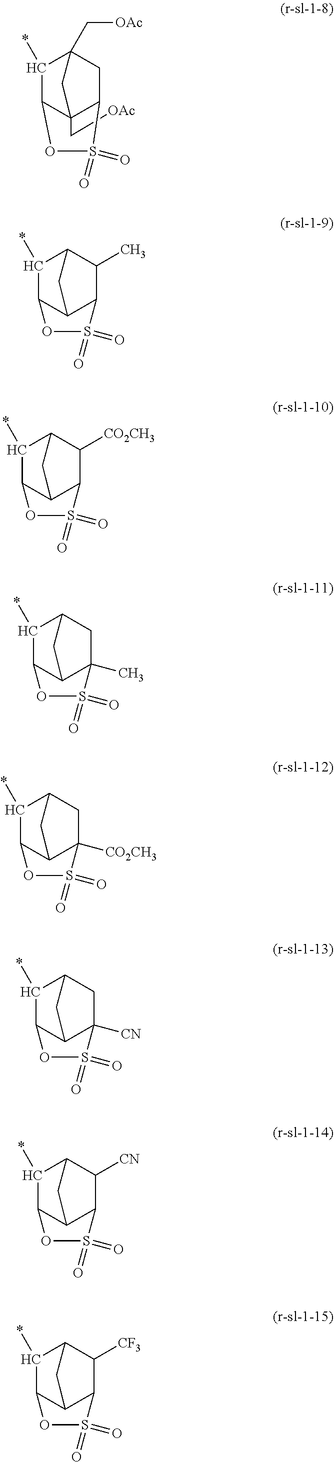

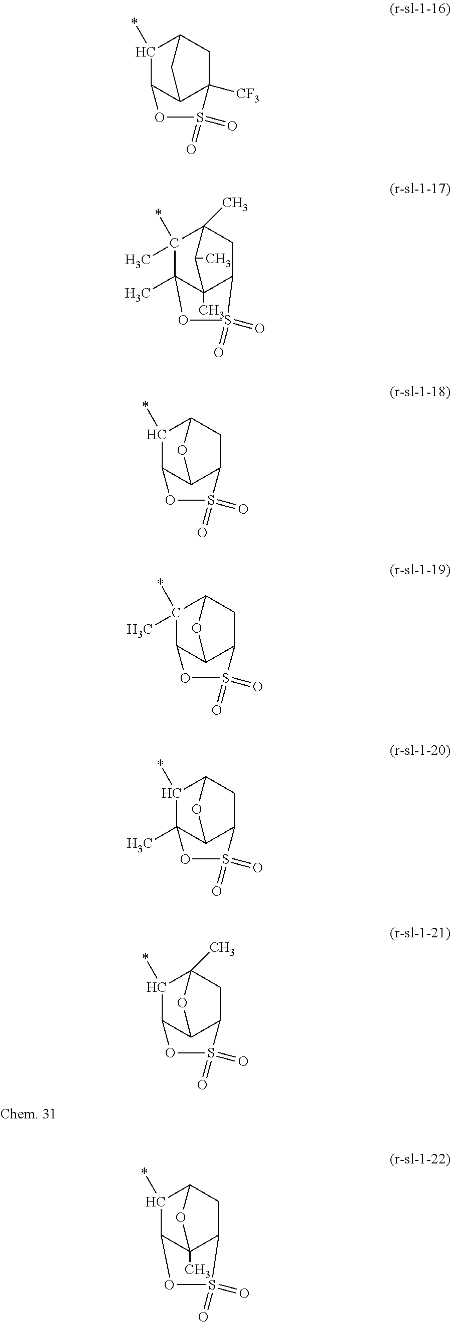

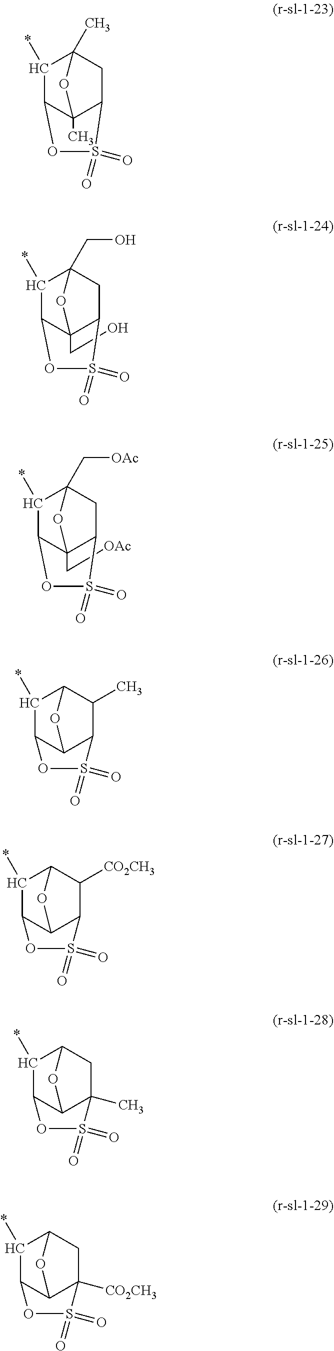

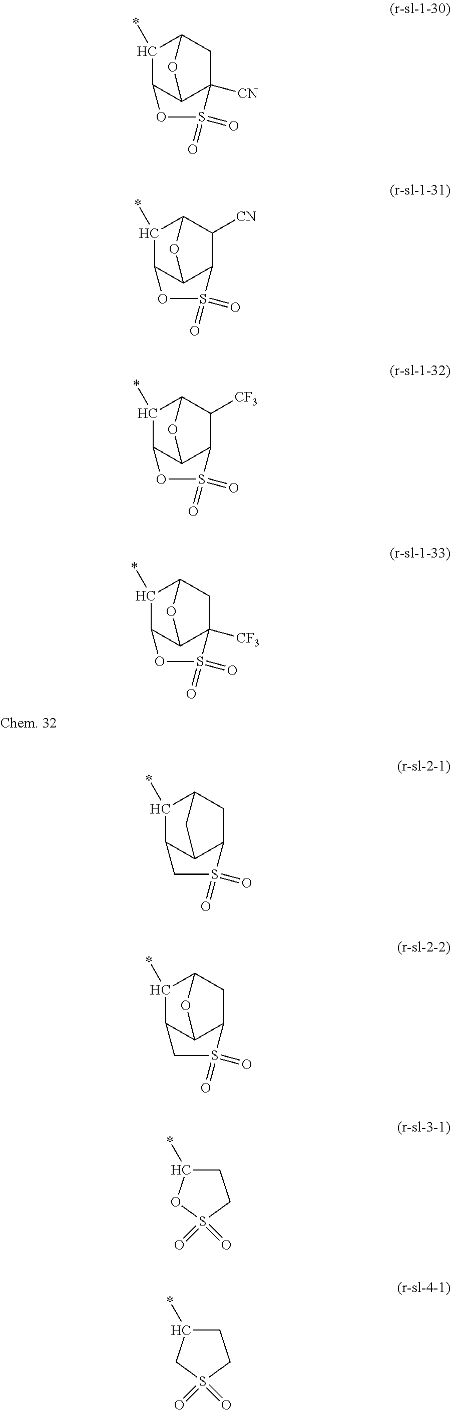

The term “carbonate-containing cyclic group” refers to a cyclic group containing a ring (a carbonate ring) containing —O—C(═O)—O— in the ring skeleton thereof. When a carbonate ring is counted as a first ring, a group having only a carbonate ring is referred to as a monocyclic group, and a group further having another ring structure is referred to as a polycyclic group regardless of the structure. The carbonate-containing cyclic group may be a monocyclic group or a polycyclic group.

The carbonate ring-containing cyclic group is not particularly limited, and any group can be used. Specific examples thereof include a group represented by any one of the following general formulae (ax3-r-1) to (ax3-r-3).

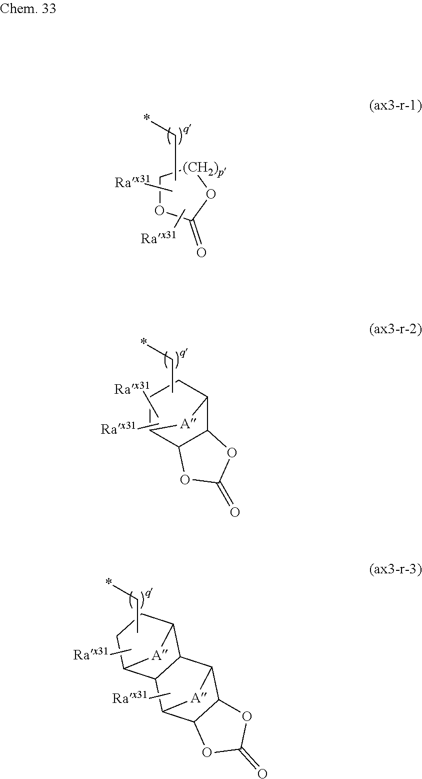

In the formulae, Ra′^{x31}'s each independently represent a hydrogen atom, an alkyl group, an alkoxy group, a halogen atom, a halogenated alkyl group, a hydroxy group, —COOR″, —OC(═O)R″, a hydroxyalkyl group, or a cyano group. R″ represents a hydrogen atom, an alkyl group, a lactone-containing cyclic group, a carbonate-containing cyclic group, or a —SO₂-containing cyclic group. A″ represents an alkylene group having 1 to 5 carbon atoms that may contain an oxygen atom or a sulfur atom, an oxygen atom, or a sulfur atom. p′ is an integer of 0 to 3, and q′ is 0 or 1. * indicates a bond.

In the general formulae (ax3-r-2) to (ax3-r-3), A″ is the same as A″ in the general formulae (a2-r-2), (a2-r-3) and (a2-r-5).

Examples of the alkyl group, alkoxy group, halogen atom, halogenated alkyl group, —COOR″, —OC(═O)R″, and hydroxyalkyl group in Ra′^{x31} include those same as those described in the description of Ra′²¹ in the general formulae (a2-r-1) to (a2-r-7).

Specific examples of the group represented by any one of the general formulae (ax3-r-1) to (ax3-r-3) are shown below.

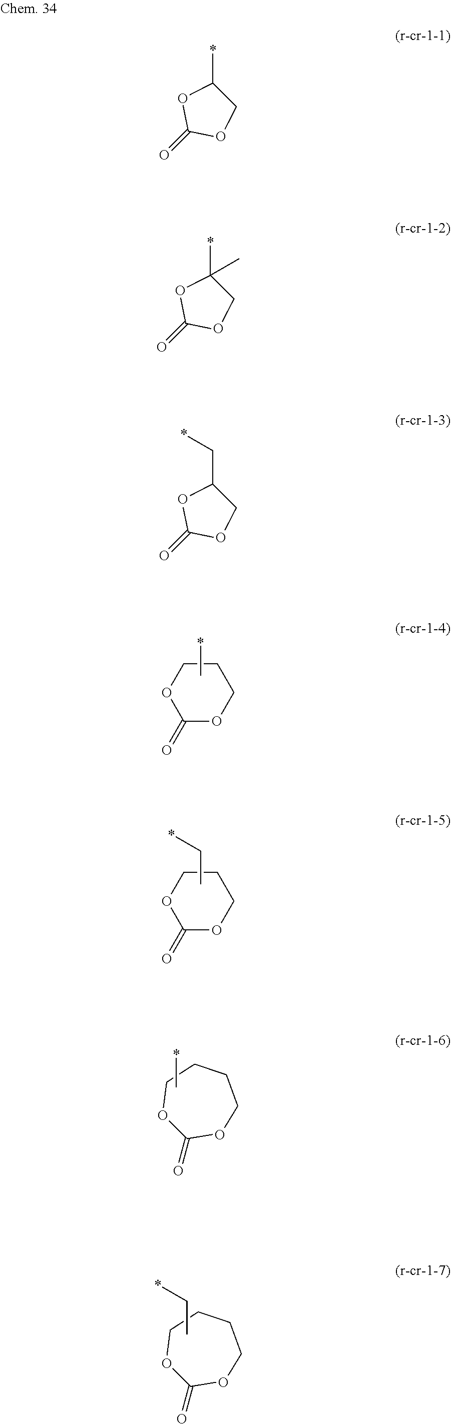

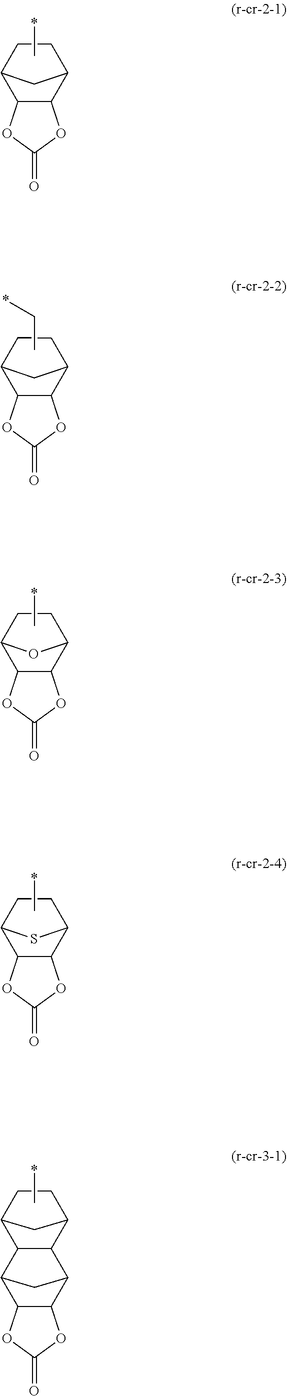

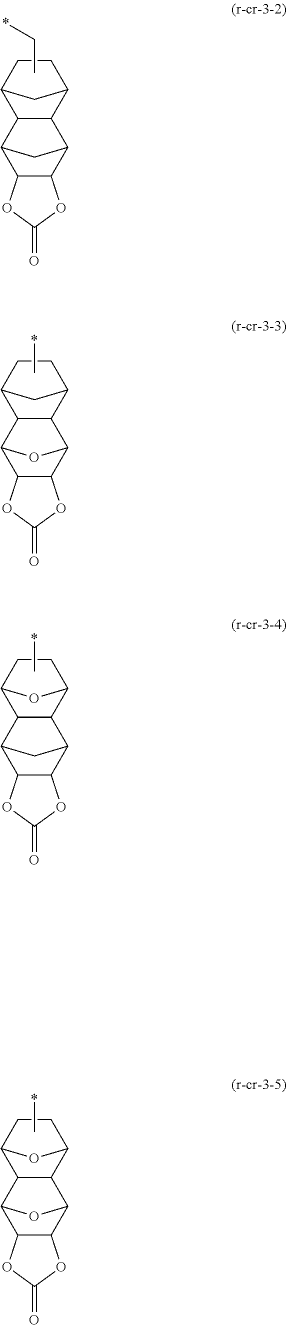

As the structural unit (a2), a structural unit derived from an acrylic ester in which a hydrogen atom bonded to a carbon atom at an α-position may be replaced by a substituent is preferred.

Such a structural unit (a2) is preferably a structural unit represented by the following general formula (a2-1).

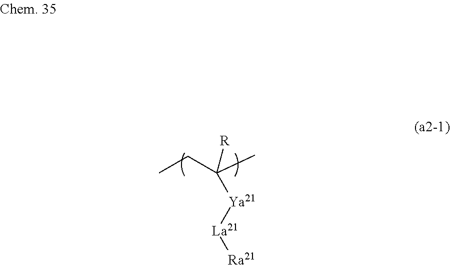

(In the general formula (a2-1), R represents a hydrogen atom, an alkyl group having 1 to 5 carbon atoms, or a halogenated alkyl group having 1 to 5 carbon atoms. Ya²¹ represents a single bond or a divalent linking group. La²¹ is-O—, —COO—, —CON(R′)—, —OCO—, —CONHCO— or —CONHCS—, R′ represents a hydrogen atom or a methyl group. When La²¹ is-O—, Ya²¹ does not represent-CO—. Ra²¹ is a lactone-containing cyclic group, a carbonate-containing cyclic group, or a —SO₂-containing cyclic group.]

In the formula (a2-1), R is the same as R in the formula (a1-1). R is preferably a hydrogen atom, an alkyl group having 1 to 5 carbon atoms, or a fluorinated alkyl group having 1 to 5 carbon atoms, and from the viewpoint of industrial availability, a hydrogen atom or a methyl group is particularly preferred.

In the formula (a2-1), the divalent linking group in Ya²¹ is not particularly limited, and suitable examples thereof include a divalent hydrocarbon group that may include a substituent, and a divalent linking group containing a heteroatom.

Divalent Hydrocarbon Group that May Include Substituent:

When Ya²¹ is a divalent hydrocarbon group that may include a substituent, the hydrocarbon group may be an aliphatic hydrocarbon group or an aromatic hydrocarbon group.

### Aliphatic Hydrocarbon Group in Ya²¹

An aliphatic hydrocarbon group means a hydrocarbon group having no aromaticity. The aliphatic hydrocarbon group may be saturated or unsaturated, and is generally preferably saturated.

Examples of the aliphatic hydrocarbon group include a linear or branched aliphatic hydrocarbon group and an aliphatic hydrocarbon group containing a ring in the structure thereof.

### Linear or Branched Aliphatic Hydrocarbon Group

The linear aliphatic hydrocarbon group preferably has 1 to 10 carbon atoms, more preferably has 1 to 6 carbon atoms, still more preferably has 1 to 4 carbon atoms, and most preferably has 1 to 3 carbon atoms.

The linear aliphatic hydrocarbon group is preferably a linear alkylene group, and specific examples thereof include a methylene group [—CH₂—], an ethylene group [—(CH₂)₂—], a trimethylene group [—(CH₂)₃—], a tetramethylene group [—(CH₂)₄—], and a pentamethylene group [—(CH₂)₅—].

The branched aliphatic hydrocarbon group preferably has 2 to 10 carbon atoms, more preferably has 3 to 6 carbon atoms, still more preferably has 3 or 4 carbon atoms, and most preferably has 3 carbon atoms.

The branched aliphatic hydrocarbon group is preferably a branched alkylene group, and specific examples thereof include alkyl alkylene groups such as alkylmethylene groups such as —CH(CH₃)—, —CH(CH₂CH₃)—, —C(CH₃)₂—, —C(CH₃)(CH₂CH₃)—, —C(CH₃)(CH₂CH₂CH₃)—, and —C(CH₂CH₃)₂—; alkyl ethylene groups such as —CH(CH₃)CH₂—, —CH(CH₃)CH(CH₃)—, —C(CH₃)₂CH₂—, —CH(CH₂CH₃)CH₂—, and —C(CH₂CH₃)₂—CH₂—; alkyl trimethylene groups such as —CH(CH₃)CH₂CH₂— and —CH₂CH(CH₃)CH₂—; and alkyl tetramethylene groups such as —CH(CH₃)CH₂CH₂CH₂— and —CH₂CH(CH₃)CH₂CH₂—. The alkyl group in the alkylalkylene group is preferably a linear alkyl group having 1 to 5 carbon atoms.

The linear or branched aliphatic hydrocarbon group may or may not include a substituent. Examples of the substituent include a fluorine atom, a fluorinated alkyl group having 1 to 5 carbon atoms and being replaced by a fluorine atom, and a carbonyl group.

### Aliphatic Hydrocarbon Group Containing Ring in Structure Thereof

Examples of the aliphatic hydrocarbon group containing a ring in the structure thereof include a cyclic aliphatic hydrocarbon group that may include a substituent containing a heteroatom in the ring structure thereof (a group in which two hydrogen atoms are removed from an aliphatic hydrocarbon ring), a group in which the cyclic aliphatic hydrocarbon group is bonded to the end of a linear or branched aliphatic hydrocarbon group, and a group in which the cyclic aliphatic hydrocarbon group is interposed in the middle of a linear or branched aliphatic hydrocarbon group. Examples of the linear or branched aliphatic hydrocarbon group include those same as described above.

The cyclic aliphatic hydrocarbon group preferably has 3 to 20 carbon atoms, and more preferably has 3 to 12 carbon atoms.

The cyclic aliphatic hydrocarbon group may be either a polycyclic group or a monocyclic group. The monocyclic alicyclic hydrocarbon group is preferably a group in which two hydrogen atoms are removed from a monocycloalkane. The monocycloalkane preferably has 3 to 6 carbon atoms, and specific examples thereof include cyclopentane and cyclohexane. The polycyclic alicyclic hydrocarbon group is preferably a group in which two hydrogen atoms are removed from a polycycloalkane. The polycycloalkane is preferably a group having 7 to 12 carbon atoms. Specific examples thereof include adamantane, norbornane, isobornane, tricyclo[5.2.1.02,6]decane, and tetracyclododecane.

The cyclic aliphatic hydrocarbon group may or may not include a substituent. Examples of the substituent include an alkyl group, an alkoxy group, a halogen atom, a halogenated alkyl group, a hydroxy group, and a carbonyl group.

The alkyl group as the substituent is preferably an alkyl group having 1 to 5 carbon atoms, and more preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group, or an ethoxy group.

Examples of the halogen atom as the substituent include a fluorine atom, a chlorine atom, a bromine atom, and an iodine atom. A fluorine atom is preferred.

Examples of the halogenated alkyl group as the substituent include a group in which a part or all of hydrogen atoms of the alkyl group are replaced by halogen atoms.

In the cyclic aliphatic hydrocarbon group, a part of carbon atoms constituting the ring structure thereof may be replaced by a substituent containing a heteroatom. The substituent containing a heteroatom is preferably-O—, —C(═O)—O—, —S—, —S(═O)₂—, or —S(═O)₂—O—.

### Aromatic Hydrocarbon Group in Ya²¹

The aromatic hydrocarbon group is a hydrocarbon group having at least one aromatic ring.

The aromatic ring is not particularly limited as long as it is of a cyclic conjugated system having 4n+2 π electrons, and may be monocyclic or polycyclic. The number of carbon atoms of the aromatic ring is preferably 5 to 30, more preferably 5 to 20, still more preferably 6 to 15, and particularly preferably 6 to 12. The number of the carbon atoms does not include the number of the carbon atoms in the substituent.

Specific examples of the aromatic ring include an aromatic hydrocarbon ring such as benzene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Specific examples of the aromatic hydrocarbon group include a group in which two hydrogen atoms are removed from the aromatic hydrocarbon ring or aromatic heterocyclic ring (an arylene group or a heteroarylene group); a group in which two hydrogen atoms are removed from an aromatic compound containing two or more aromatic rings (for example, biphenyl or fluorene); and a group in which one hydrogen atom of a group in which one hydrogen atom is removed from the aromatic hydrocarbon ring or aromatic heterocyclic ring (an aryl group or a heteroaryl group) is replaced by an alkylene group (for example, a group in which one hydrogen atom is further removed from the aryl group in the arylalkyl group, such as a benzyl group, a phenethyl group, a 1-naphthylmethyl group, a 2-naphthylmethyl group, a 1-naphthylethyl group, or a 2-naphthylethyl group). The number of carbon atoms of the alkylene group bonded to the aryl group or the heteroaryl group is preferably 1 to 4, more preferably 1 to 2, and particularly preferably 1.

In the aromatic hydrocarbon group, a hydrogen atom of the aromatic hydrocarbon group may be replaced by a substituent. For example, a hydrogen atom bonded to an aromatic ring in the aromatic hydrocarbon group may be replaced by a substituent. Examples of the substituent include an alkyl group, an alkoxy group, a halogen atom, a halogenated alkyl group, and a hydroxy group.

The alkyl group as the substituent is preferably an alkyl group having 1 to 5 carbon atoms, and more preferably a methyl group, an ethyl group, a propyl group, an n-butyl group, or a tert-butyl group.

Examples of the alkoxy group, halogen atom, and halogenated alkyl group as the substituent include those exemplified as the substituent for replacing the hydrogen atom in the cyclic aliphatic hydrocarbon group.

### Divalent Linking Group Containing Heteroatom:

When Ya²¹ is a divalent linking group containing a heteroatom, preferred examples of the linking group include-O—, —C(═O)—O—, —O—C(═O)—, —C(═O)—, —O—C(═O)—O—, —C(═O)—NH—, —NH—, —NH—C(═NH)—(H may be replaced by a substituent such as an alkyl group or an acyl group), —S—, —S(═O)₂—, —S(═O)₂—O—, and a group represented by a formula —Y₂₁—O—Y₂₂—, —Y₂₁—O—, —Y₂₁_C(═O)—O—, —C(═O)—O—Y₂₁—, —[Y₂₁—C(═O)—O]m″—Y₂₂, —Y₂₁—O—C(═O)—Y₂₂— or —Y₂₁—S(═O) 2-O—Y₂₂-[in the formulae, Y₂₁ and Y₂₂ each independently represent a divalent hydrocarbon group that may include a substituent, O is an oxygen atom, and m″ is an integer of 1 to 3].

When the divalent linking group containing a heteroatom is-C(═O)—NH—, —NH—, or —NH—C(═NH)—, H thereof may be replaced by a substituent such as an alkyl group or an acyl group. The substituent (an alkyl group, an acyl group, or the like) preferably has 1 to 10 carbon atoms, more preferably has 1 to 8 carbon atoms, and particularly preferably has 1 to 5 carbon atoms.

In the formulae-Y₂₁—O—Y₂₂—, —Y₂₁—O—, —Y₂₁—C(═O)—O—, —C(═O)—O—Y₂₁—, —[Y₂₁—C(═O)—O]_{m″}—Y₂₂—, —Y₂₁—O—C(═O)—Y₂₂— or —Y₂₁—S(═O) 2-O—Y₂₂—, Y₂₁ and Y₂₂ each independently represent a divalent hydrocarbon group that may include a substituent. Examples of the divalent hydrocarbon group include those same as those described above for the divalent linking group in Ya²¹ (the divalent hydrocarbon group that may include a substituent).

Y₂₁ is preferably a linear aliphatic hydrocarbon group, more preferably a linear alkylene group, still more preferably a linear alkylene group having 1 to 5 carbon atoms, and particularly preferably a methylene group or an ethylene group.

Y₂₂ is preferably a linear or branched aliphatic hydrocarbon group, and more preferably a methylene group, an ethylene group, or an alkylmethylene group. The alkyl group in the alkylmethylene group is preferably a linear alkyl group having 1 to 5 carbon atoms, more preferably a linear alkyl group having 1 to 3 carbon atoms, and most preferably a methyl group.

In the group represented by the formula —[Y₂₁—C(═O)—O]_{m″}—Y₂₂—, m″ is an integer of 0 to 3, preferably an integer of 0 to 2, more preferably 0 or 1, and particularly preferably 1. That is, the group represented by the formula-[Y₂₁—C(═O)—O]_{m″}—Y₂₂—, a group represented by the formula —Y₂₁—C(═O)—O—Y₂₂— is particularly preferred. Among them, a group represented by the formula —(CH₂) a′—C(═O)—O—(CH₂)_{b}′— is preferred. In the formula, the symbol a′ is an integer of 1 to 10, preferably an integer of 1 to 8, more preferably an integer of 1 to 5, still more preferably 1 or 2, and most preferably 1. The symbol b′ is an integer of 1 to 10, preferably an integer of 1 to 8, more preferably an integer of 1 to 5, still more preferably 1 or 2, and most preferably 1.

Among them, Ya²¹ is preferably a single bond, an ester bond [—C(═O)—O—], an ether bond (—O—), a linear or branched alkylene group, or a combination thereof.

In the formula (a2-1), Ra²¹ represents a lactone-containing cyclic group, a —SO₂-containing cyclic group, or a carbonate-containing cyclic group.

Suitable examples of the lactone-containing cyclic group, the —SO₂-containing cyclic group, and the carbonate-containing cyclic group in Ra²¹ include a group represented by any one of the general formulae (a2-r-1) to (a2-r-7), a group represented by any one of general formulae (a5-r-1) to (a5-r-4), and a group represented by any one of general formulae (ax3-r-1) to (ax3-r-3).

In the formula (a2-1), among the above, Ra²¹ is preferably a lactone-containing cyclic group or a —SO₂-containing cyclic group, more preferably a group represented by the general formula (a2-r-1), (a2-r-2), (a2-r-6), or (a5-r-1), and still more preferably a group represented by any one of the general formulae (a2-r-1) and (a2-r-2).

Specifically, any one of the groups represented by the chemical formulae (r-lc-1-1) to (r-lc-1-7), (r-lc-2-1) to (r-lc-2-18), and (r-lc-6-1) is preferred, and any one of the groups represented by the chemical formula (r-lc-1-1), (r-lc-2-1), and (r-lc-2-12) is more preferred.

The structural unit (a2) contained in the component (A1) may be used alone or in combination of two or more kinds thereof.

When the component (A1) contains the structural unit (a2), a proportion of the structural unit (a2) is preferably 20 mol % to 80 mol %, more preferably 30 mol % to 70 mol %, and particularly preferably 40 mol % to 60 mol %, with respect to the total (100 mol %) of all structural units constituting the component (A1).

When the proportion of the structural unit (a2) is equal to or larger than a preferred lower limit value, the effects of containing the structural unit (a2) can be sufficiently obtained due to the above-described effects. When the proportion of the structural unit (a2) is equal to or less than an upper limit value, a balance with other structural units can be achieved, and various lithography properties become excellent.

### Structural Unit (a3)

The component (A1) may contain, in addition to the structural unit (a1), a structural unit (a3) containing a polar group-containing aliphatic hydrocarbon group (provided that the structural unit (a1) or the structural unit (a2) is excluded). When the component (A1) contains the structural unit (a3), hydrophilicity of the component (A) is increased, which contributes to improvement of the resolution. The acid diffusion length can be appropriately adjusted.

Examples of the polar group include a hydroxy group, a cyano group, a carboxy group, and a hydroxyalkyl group in which some hydrogen atoms of an alkyl group are replaced by fluorine atoms, and a hydroxy group is particularly preferred.

Examples of the aliphatic hydrocarbon group include a linear or branched hydrocarbon group having 1 to 10 carbon atoms (preferably an alkylene group) and a cyclic aliphatic hydrocarbon group (a cyclic group). The cyclic group may be a monocyclic group or a polycyclic group, and for example, can be appropriately selected from a number of groups proposed for a resist composition for ArF excimer lasers.

When the cyclic group is a monocyclic group, the number of carbon atoms thereof is more preferably 3 to 10. Among them, a structural unit derived from an acrylic ester containing an aliphatic monocyclic group that contains a hydroxy group, a cyano group, a carboxy group, or a hydroxyalkyl group in which a part of hydrogen atoms of the alkyl group are replaced by fluorine atoms is more preferred. Examples of the monocyclic group include a group in which two or more hydrogen atoms are removed from a monocycloalkane. Specific examples thereof include a group in which two or more hydrogen atoms are removed from a monocycloalkane such as cyclopentane, cyclohexane, and cycclooctane. Among the monocyclic group, a group in which two or more hydrogen atoms are removed from cyclopentane and a group in which two or more hydrogen atoms are removed from cyclohexane are industrially preferred.

When the cyclic group is a polycyclic group, the number of carbon atoms of the polycyclic group is more preferably 7 to 30. Among them, a structural unit derived from an acrylic ester containing an aliphatic polycyclic group that contains a hydroxy group, a cyano group, a carboxy group, or a hydroxyalkyl group in which a part of hydrogen atoms of the alkyl group are replaced by fluorine atoms is more preferred. Examples of the polycyclic group include a group in which two or more hydrogen atoms are removed from a bicycloalkane, tricycloalkane, tetracycloalkane, or the like. Specific examples thereof include a group in which two or more hydrogen atoms are removed from a polycycloalkane such as adamantane, norbornane, isobornane, tricyclodecane, or tetracyclododecane. Among the polycyclic groups, a group in which two or more hydrogen atoms are removed from adamantane, a group in which two or more hydrogen atoms are removed from norbornane, and a group in which two or more hydrogen atoms are removed from tetracyclododecane are industrially preferred.

There are no particular limitations on the structural unit (a3), and any structural unit can be used as long as it contains a polar group-containing aliphatic hydrocarbon group.

As the structural unit (a3), a structural unit derived from an acrylic ester in which the hydrogen atom bonded to the carbon atom on the α-position may be replaced by a substituent and containing a polar group-containing aliphatic hydrocarbon group is preferred.

When the hydrocarbon group in the polar group-containing aliphatic hydrocarbon group is a linear or branched hydrocarbon group having 1 to 10 carbon atoms, the structural unit (a3) is preferably a structural unit derived from a hydroxy ethyl ester of acrylic acid.

When the hydrocarbon group in the polar group-containing aliphatic hydrocarbon group is a polycyclic group, preferred examples of the structural unit (a3) include a structural unit represented by the following formula (a3-1), a structural unit represented by the following formula (a3-2), and a structural unit represented by the following formula (a3-3). When the hydrocarbon group in the polar group-containing aliphatic hydrocarbon group is a monocyclic group, preferred examples of the structural unit (a3) include a structural unit represented by the following formula (a3-4).

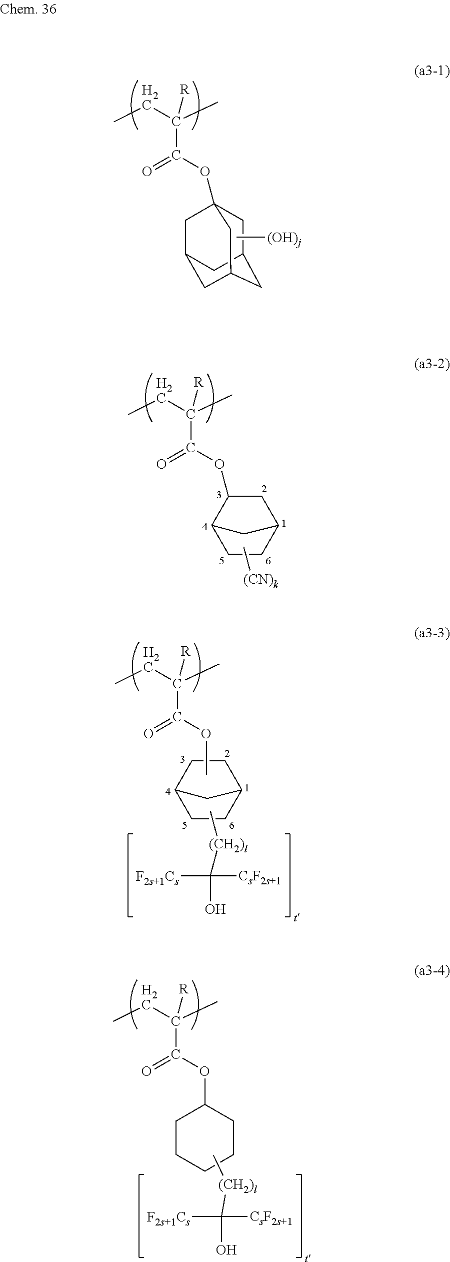

In the formula, R is the same as above, j is an integer of 1 to 3, k is an integer of 1 to 3, t′ is an integer of 1 to 3, 1 is an integer of 0 to 5, and s is an integer of 1 to 3.

In the formula (a3-1), j is preferably 1 or 2, and more preferably 1. When j is 2, the hydroxy groups are preferably bonded to the 3-position and 5-position of the adamantyl group. When j is 1, the hydroxy group is preferably bonded to the 3-position of the adamantyl group.

It is preferable that j be 1, and it is particularly preferable that the hydroxy group be bonded to the 3-position of the adamantyl group.

In the formula (a3-2), k is preferably 1. The cyano group is preferably bonded to the 5-position or 6-position of a norbornyl group.

In the formula (a3-3), t′ is preferably 1. 1 is preferably 1. s is preferably 1. A 2-norbornyl group or a 3-norbornyl group is preferably bonded to the end of the carboxy group of acrylic acid. The fluorinated alkyl alcohol is preferably bonded to the 5-position or 6-position of the norbornyl group.

In the formula (a3-4), t′ is preferably 1 or 2. 1 is preferably 0 or 1. s is preferably 1. The fluorinated alkyl alcohol is preferably bonded to the 3-position or 5-position of a cyclohexyl group.

The structural unit (a3) contained in the component (A1) may be used alone or in combination of two or more kinds thereof.

When the component (A1) contains the structural unit (a3), a proportion of the structural unit (a3) is preferably 1 mol % to 30 mol %, more preferably 2 mol % to 25 mol %, and still more preferably 5 mol % to 20 mol %, with respect to the total (100 mol %) of all structural units constituting the component (A1).

When the proportion of the structural unit (a3) is equal to or more than the preferred lower limit value, the effects of containing the structural unit (a3) can be sufficiently obtained due to the above-described effects. When the proportion of the structural unit (a3) is equal to or less than the preferred upper limit value, a balance with other structural units can be achieved, and various lithography properties become excellent.

Structural Unit (a10)

The component (A1) may further contain a structural unit (a10) represented by a general formula (a10-1).

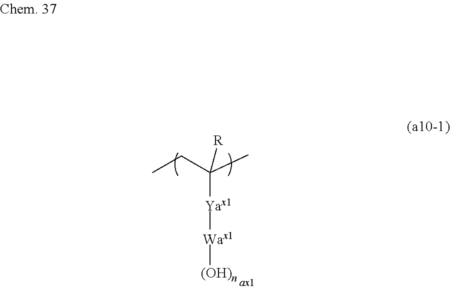

In the formula, R is a hydrogen atom, an alkyl group having 1 to 5 carbon atoms, or a halogenated alkyl group having 1 to 5 carbon atoms. Ya^{x1} is a single bond or a divalent linking group. Wa^{x1} is a (nₐₓ₁+1)-valent aromatic hydrocarbon group. nₐₓ₁ is an integer of 1 or more.

In the formula (a10-1), R is the same as R in the formula (a1-1). R is preferably a hydrogen atom, an alkyl group having 1 to 5 carbon atoms, or a fluorinated alkyl group having 1 to 5 carbon atoms, and from the viewpoint of industrial availability, a hydrogen atom or a methyl group is particularly preferred.

In the above formula (a10-1), Ya^{x1} represents a single bond or a divalent linking group.

In the above chemical formula, examples of the divalent linking group in Ya^{x1} include those same as those described for the divalent linking group in Ya²¹ in the above formula (a2-1).

Ya^{x1} is preferably a single bond, an ester bond [—C(═O)—O—, —O—C(═O)—], an ether bond (—O—), a linear or branched alkylene group, or a combination thereof, more preferably a single bond or an ester bond [—C(═O)—O—, —O—C(═O)—], and even more preferably a single bond.

In the above formula (a10-1), Wa^{x1} is a (nₐₓ₁+1)-valent aromatic hydrocarbon group.

Examples of the aromatic hydrocarbon group in Wa^{x1} include a group in which (nₐₓ₁+1) hydrogen atoms are removed from an aromatic ring. The aromatic ring here is not particularly limited as long as it is of a cyclic conjugated system having 4n+2 π electrons, and may be monocyclic or polycyclic. The number of carbon atoms of the aromatic ring is preferably 5 to 30, more preferably 5 to 20, still more preferably 6 to 15, and particularly preferably 6 to 12. Specific examples of the aromatic ring include an aromatic hydrocarbon ring such as benzene, naphthalene, anthracene, and phenanthrene; and an aromatic heterocyclic ring in which a part of carbon atoms constituting the aromatic hydrocarbon ring is replaced by a heteroatom. Examples of the heteroatom in the aromatic heterocyclic ring include an oxygen atom, a sulfur atom, and a nitrogen atom. Specific examples of the aromatic heterocyclic ring include a pyridine ring and a thiophene ring.

Examples of the aromatic hydrocarbon group in Wa^{x1} include a group in which (nₐₓ₁+1) hydrogen atoms are removed from an aromatic compound containing two or more aromatic rings (for example, biphenyl and fluorene).

Among them, Wa^{x1} is preferably a group in which (nₐₓ₁+1) hydrogen atoms are removed from benzene, naphthalene, anthracene, or biphenyl, more preferably a group in which (nₐₓ₁+1) hydrogen atoms are removed from benzene or naphthalene, and still more preferably a group in which (nₐₓ₁+1) hydrogen atoms are removed from benzene.

The aromatic hydrocarbon group in Wa^{x1} may or may not have a substituent, but preferably has no substituent. Examples of the substituent include an alkyl group, an alkoxy group, a halogen atom, and a halogenated alkyl group. Examples of the alkyl group, alkoxy group, halogen atom, and halogenated alkyl group as the substituent include groups same as those described for the substituent of the cyclic aliphatic hydrocarbon group in Ya^{x1}. When Wa^{x1} is a group in which (nₐₓ₁+1) hydrogen atoms are removed from an aromatic ring, the substituent is a substituent for substituting a hydrogen atom in the group in which (nₐₓ₁+1) hydrogen atoms are removed from an aromatic ring. The substituent is preferably a linear or branched alkyl group having 1 to 5 carbon atoms, more preferably a linear or branched alkyl group having 1 to 3 carbon atoms, still more preferably an ethyl group or a methyl group, and particularly preferably a methyl group.

In the above formula (a10-1), nₐₓ₁ is an integer of 1 or more, preferably an integer of 1 to 10, more preferably an integer of 1 to 5, still more preferably 1, 2 or 3, and particularly preferably 1 or 2. Among them, nₐₓ₁ is preferably 1.

Specific examples of the structural unit (a10) represented by the formula (a10-1) are shown below.

In the following formulae, R^{α} represents a hydrogen atom, a methyl group, or a trifluoromethyl group.

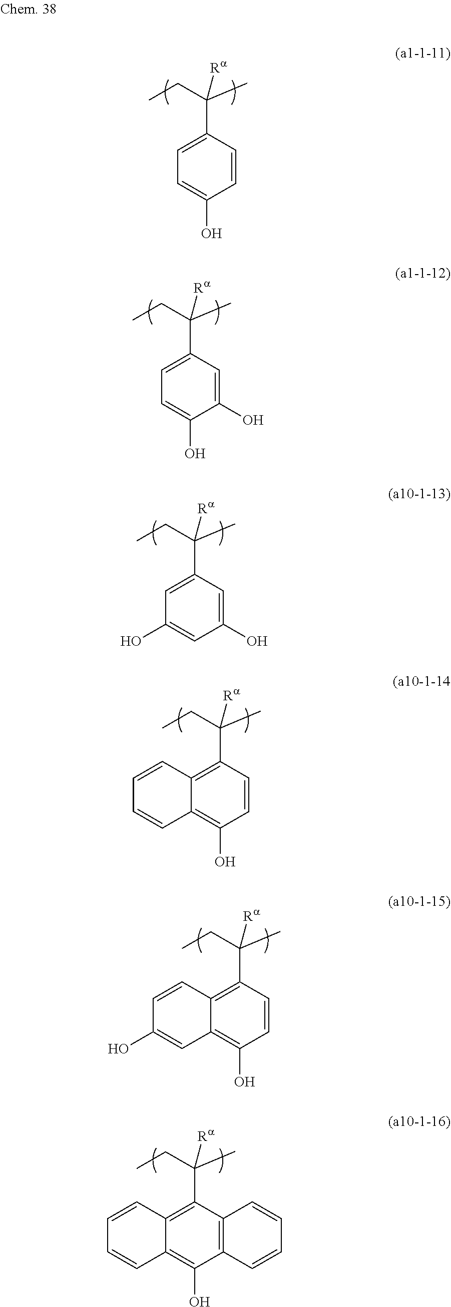

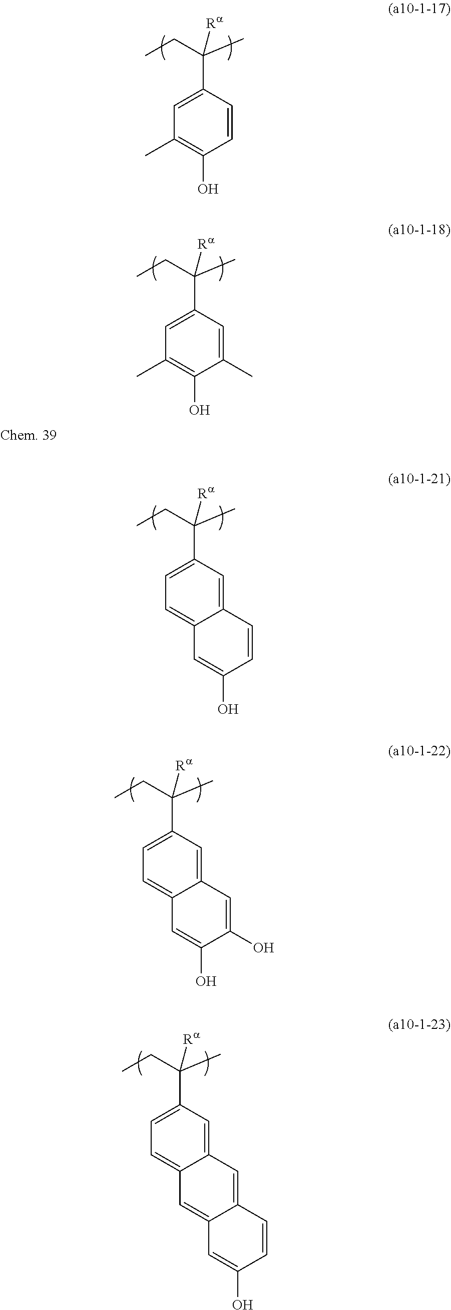

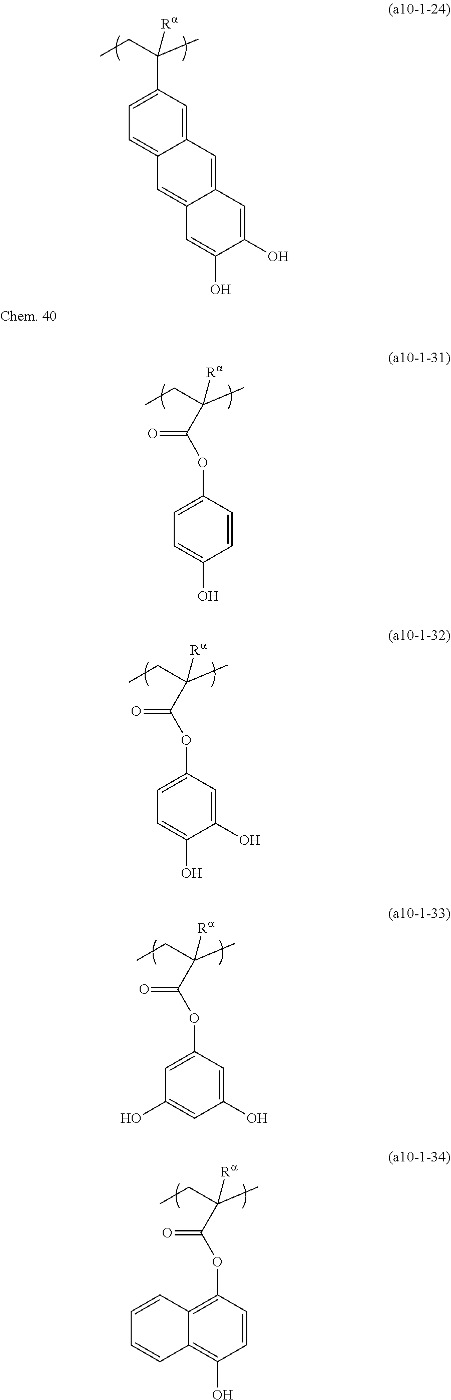

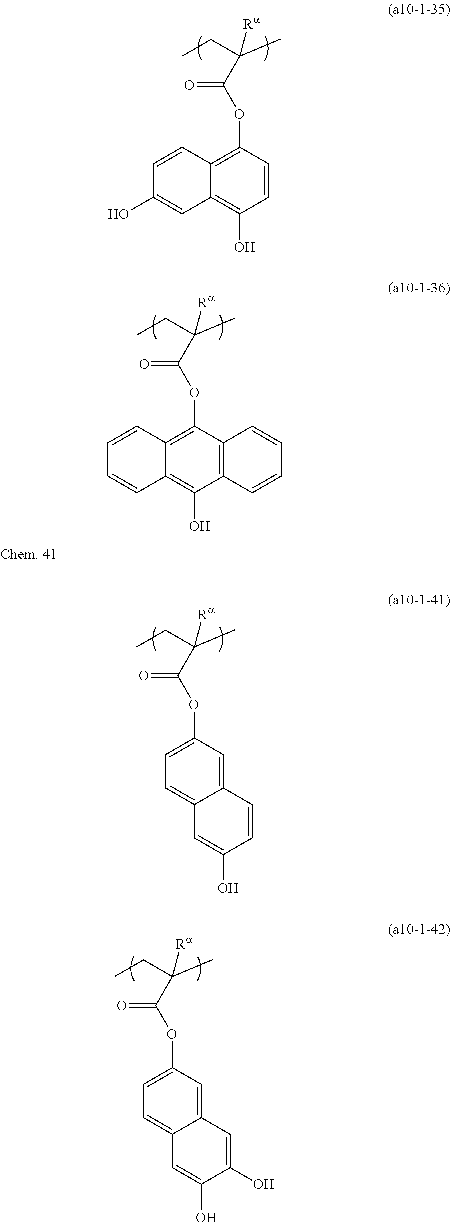

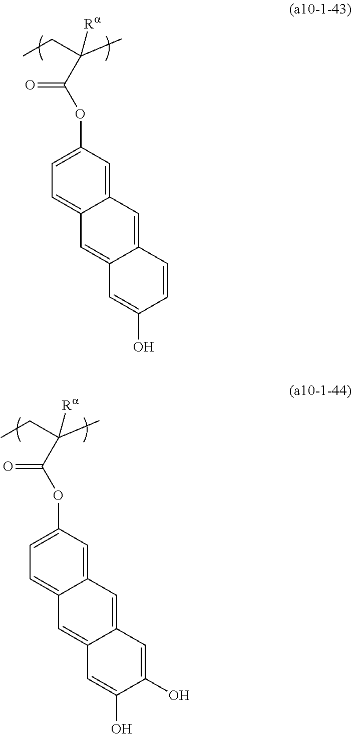

The structural unit (a10) contained in the component (A1) may be used alone or in combination of two or more kinds thereof.

When the component (A1) contains the structural unit (a10), a proportion of the structural unit (a10) in the component (A1) is preferably 5 mol % to 80 mol %, more preferably 5 mol % to 75 mol %, and still more preferably 10 mol % to 70 mol %, with respect to the total (100 mol %) of all structural units constituting the component (A1).

When the proportion of the structural unit (a10) is equal to or more than the lower limit value, the sensitivity can be further enhanced. On the other hand, when the proportion of the structural unit (a10) is equal to or less than the upper limit value, a balance with other structural units is easily achieved.

### Structural Unit (st)

The component (A1) may contain, in addition to the structural unit (a1), a structural unit (st) derived from styrene or a styrene derivative.

The structural unit (st) is a structural unit derived from styrene or a styrene derivative. The term “structural unit derived from styrene” means a structural unit formed by cleavage of an ethylenic double bond of styrene. The term “structural unit derived from a styrene derivative” means a structural unit formed by cleavage of an ethylenic double bond of a styrene derivative.

The term “styrene derivative” means a compound in which at least a part of hydrogen atoms of styrene is replaced by a substituent. Examples of the styrene derivative include those in which the hydrogen atom of styrene on the α-position is replaced by a substituent, those in which one or more hydrogen atoms of a benzene ring of styrene are replaced by a substituent, and those in which the hydrogen atom of styrene on the α-position and one or more hydrogen atoms of the benzene ring are replaced by a substituent.

Examples of the substituent for replacing the hydrogen atom of styrene on the α-position include an alkyl group having 1 to 5 carbon atoms and a halogenated alkyl group having 1 to 5 carbon atoms.

The alkyl group having 1 to 5 carbon atoms is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

The halogenated alkyl group having 1 to 5 carbon atoms is a group in which part or all of hydrogen atoms of the alkyl group having 1 to 5 carbon atoms are replaced by halogen atoms. Examples of the halogen atom include a fluorine atom, a chlorine atom, a bromine atom, and an iodine atom. A fluorine atom is particularly preferred.

The substituent for replacing the hydrogen atom of styrene on the α-position is preferably an alkyl group having 1 to 5 carbon atoms or a fluorinated alkyl group having 1 to 5 carbon atoms, more preferably an alkyl group having 1 to 3 carbon atoms or a fluorinated alkyl group having 1 to 3 carbon atoms, and still more preferably a methyl group from the viewpoint of industrial availability.

Examples of the substituent for replacing the hydrogen atom of the benzene ring of styrene include an alkyl group, an alkoxy group, a halogen atom, and a halogenated alkyl group.

The alkyl group as the substituent is preferably an alkyl group having 1 to 5 carbon atoms, and more preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group, or an ethoxy group.

Examples of the halogen atom as the substituent include a fluorine atom, a chlorine atom, a bromine atom, and an iodine atom. A fluorine atom is preferred.

Examples of the halogenated alkyl group as the substituent include a group in which a part or all of hydrogen atoms of the alkyl group are replaced by halogen atoms.

The substituent for replacing the hydrogen atom of the benzene ring of styrene is preferably an alkyl group having 1 to 5 carbon atoms, more preferably a methyl group or an ethyl group, and still more preferably a methyl group.

The structural unit (st) is preferably a structural unit derived from styrene or a structural unit derived from a styrene derivative in which the hydrogen atom of styrene at the α-position is replaced by an alkyl group having 1 to 5 carbon atoms or a halogenated alkyl group having 1 to 5 carbon atoms, more preferably a structural unit derived from styrene or a structural unit derived from a styrene derivative in which the hydrogen atom of styrene at the α-position is replaced by a methyl group, and still more preferably a structural unit derived from styrene.

The structural unit (st) contained in the component (A1) may be used alone or in combination of two or more kinds thereof.

When the component (A1) contains the structural unit (st), a proportion of the structural unit (st) is preferably 1 mol % to 30 mol %, and more preferably 3 mol % to 30 mol %, with respect to the total (100 mol %) of all structural units constituting the component (A1).

### <<Other Structural Units>>

The component (A1) may contain other structural units other than the structural unit (a1), the structural unit (a2), the structural unit (a3), the structural unit (a10), and the structural unit (st) described above.

Examples of the other structural units include a structural unit (a8) containing a non-acid-dissociable aliphatic cyclic group. As the structural unit (a8), a large number of related-art known structural units used in a resin component of a resist composition can be used.

### <<Acid Generating Agent Component (Component (B))>>

The resist composition according to the embodiment of the present invention contains, in addition to the component (A), the acid generating agent component (B) (hereinafter, also referred to as “component (B)”) that generates an acid upon exposure, and the acid generating agent component (B) contains an acid generating agent (B1) (hereinafter, also referred to as “component (B1)”) composed of a compound represented by the following general formula (b1-1).

In the present embodiment, the acid generating agent component (B) contains a compound represented by the following general formula (b1-1) (hereinafter, may be referred to as “component (B1)”).

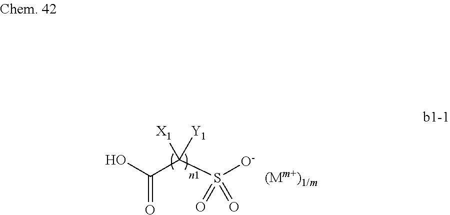

In the general formula (b1-1), X₁ and Y₁ each independently represent a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group. X₁ and Y₁ may be bonded to each other to form a ring. n1 represents an integer of 1 to 10. m is an integer of 1 or more. M^{m+} represents an m-valent organic cation.

The component (B) does not contain any compound that falls under the PFAS. The component (B) contains only compounds that do not fall under the PFAS. That is, the component (B) does not contain any compound having a trifluoromethyl group or any compound having a difluoromethylene group. Further, it is preferable that the component (B) does not contain fluorine atoms.

Anion Moiety (HO—(C═O)—(CX₁Y₁)ₙ₁—SO₃⁻)

In the general formula (b1-1), X₁ and Y₁ each independently represent a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group.

n1 preferably represents 1 or 2, and more preferably represents 1.

Examples of the aliphatic hydrocarbon group represented by X₁ and Y₁ include a linear alkyl group, a branched alkyl group, a polycyclic aliphatic hydrocarbon group, and a monocyclic aliphatic hydrocarbon group.

The linear alkyl group preferably has 1 to 5 carbon atoms, more preferably has 1 to 4 carbon atoms, and still more preferably has 1 or 2 carbon atoms. Specific examples thereof include a methyl group, an ethyl group, an n-propyl group, an n-butyl group, and an n-pentyl group. Among them, a methyl group, an ethyl group, or an n-butyl group is preferred, and a methyl group or an ethyl group is more preferred.

The branched alkyl group preferably has 3 to 10 carbon atoms, and more preferably has 3 to 5 carbon atoms. Specific examples thereof include an isopropyl group, an isobutyl group, a tert-butyl group, an isopentyl group, a neopentyl group, a 1,1-diethylpropyl group, and a 2,2-dimethylbutyl group. An isopropyl group is preferred.

The aliphatic hydrocarbon group which is a monocyclic group is preferably a group in which one hydrogen atom is removed from a monocycloalkane. The monocycloalkane preferably has 3 to 12 carbon atoms, more preferably 3 to 6 carbon atoms, and specific examples thereof include cyclopropane, cyclobutane, cyclopentane, and cyclohexane.

The aliphatic hydrocarbon group which is a polycyclic group is preferably a group in which one hydrogen atom is removed from a polycycloalkane. The polycycloalkane is preferably a group having 7 to 12 carbon atoms, and specific examples thereof include adamantane, norbornane, isobornane, tricyclo[5.2.1.02,6]decane, and tetracyclododecane.

The aromatic hydrocarbon group represented by X₁ and Y₁ preferably has 5 to 30 carbon atoms, more preferably 5 to 25, still more preferably 5 to 20, particularly preferably 6 to 15, and most preferably 6 to 12. Examples thereof include a group in which one hydrogen atom is removed from an aromatic hydrocarbon ring such as benzene, biphenyl, fluorene, naphthalene, anthracene, and phenanthrene.

Examples of the heterocyclic group represented by X₁ and Y₁ include an aromatic heterocyclic group in which a part of carbon atoms constituting the aromatic hydrocarbon group are replaced by a heteroatom. Examples of the heteroatom in the aromatic heterocyclic group include an oxygen atom, a sulfur atom, and a nitrogen atom. Examples of the heterocyclic group include a group in which one hydrogen atom is removed from an aromatic heterocyclic ring such as furan, benzofuran, thiophene, benzothiophene, pyrrole, pyrazole, imidazole, oxadiazole, indole, carbazole, pyrroloimidazole, pyrrolopyrazole, pyrrolopyrrole, thienopyrrole, thienothiophene, fluoropyrrole, furan, thienofurane, benzisoxazole, benzisothiazole, benzimidazole, pyridine, pyrazine, pyridazine, pyrimidine, and triazine.

Examples of the electron-withdrawing group represented by X₁ and Y₁ include a hydroxy group, a carbonyl group, a nitro group, a cyano group, a carboxy group, an acyl group, a dialkylphosphono group, a diarylphosphono group, an alkylsulfonyl group, a cycloalkylsulfonyl group, an arylsulfonyl group, a sulfonyloxy group, an acylthio group, a sulfamoyl group, a thiocyanate group, and a thiocarbonyl group.

Among them, as the electron-withdrawing group, a hydroxy group, a carbonyl group, a nitro group, a cyano group, a carboxy group, an acyl group, a dialkylphosphono group, a diarylphosphono group, and an alkylsulfonyl group are preferred, and a nitro group and a cyano group are more preferred.

When X₁ and Y₁ are bonded to each other to form a ring, the cyclic group is preferably a cyclic hydrocarbon group. Examples of the cyclic hydrocarbon group include an aliphatic hydrocarbon group which is a monocyclic group, an aliphatic hydrocarbon group which is a polycyclic group, and an aromatic hydrocarbon group. An aliphatic hydrocarbon group which is a monocyclic group is preferred.

When X₁ and Y₁ are bonded to each other to form a ring, the aliphatic hydrocarbon group which is a monocyclic group is preferably a group in which two hydrogen atoms are removed from a monocycloalkane. The monocycloalkane include those same as described above.

When X₁ and Y₁ are bonded to each other to form a ring, the aliphatic hydrocarbon group which is a polycyclic group is preferably a group in which two hydrogen atoms are removed from a polycycloalkane, and examples of the polycycloalkane include those same as described above.

X₁ and Y₁ may each independently have a substituent, and examples of the substituent include an alkyl group, an alkoxy group, a hydroxy group, a carbonyl group, a carboxy group, and a sulfo group.

The alkyl group as the substituent is preferably an alkyl group having 1 to 5 carbon atoms, more preferably a methyl group, an ethyl group, a propyl group, an n-butyl group, or a tert-butyl group, and still more preferably a methyl group. The alkyl group as the substituent may further have a substituent such as a carboxy group or —SO₃ (M^{m+}) 1/m (wherein m is an integer of 1 or more, and M^{m+} represents an m-valent organic cation).

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and still more preferably a methoxy group, or an ethoxy group.

In the formula (b1-1), it is preferable that X₁ and Y₁ each independently represent a hydrogen atom, an aliphatic hydrocarbon group, or an aromatic hydrocarbon group, and it is more preferable that X₁ and Y₁ each independently represent a hydrogen atom or an aliphatic hydrocarbon group. Among them, it is preferable that X₁ and Y₁ each independently represent a hydrogen atom, a group in which one hydrogen atom is removed from cyclohexane, or a group in which one hydrogen atom is removed from benzene, and it is more preferable that X₁ and Y₁ each independently represent a hydrogen atom or a group in which one hydrogen atom is removed from cyclohexane.

In the formula (b1-1), at least one of X₁ and Y₁ is preferably a hydrogen atom.

In the above formula (b1-1), X₁ is preferably an aliphatic hydrocarbon group from the viewpoint of providing the component (B) with excellent solvent solubility and ultraviolet light transmittance.

The compound represented by the general formula (b1-1) preferably does not contain a fluorine atom.

Specific examples of the anion moiety in the component (B1) are shown below. The anion moiety in the component (B1) is not limited to these specific examples. In the following specific examples, “1-11” indicates that the number of repetitions of the repeating unit represented in [ ] is 1 to 11. “Ph” represents a phenyl group.

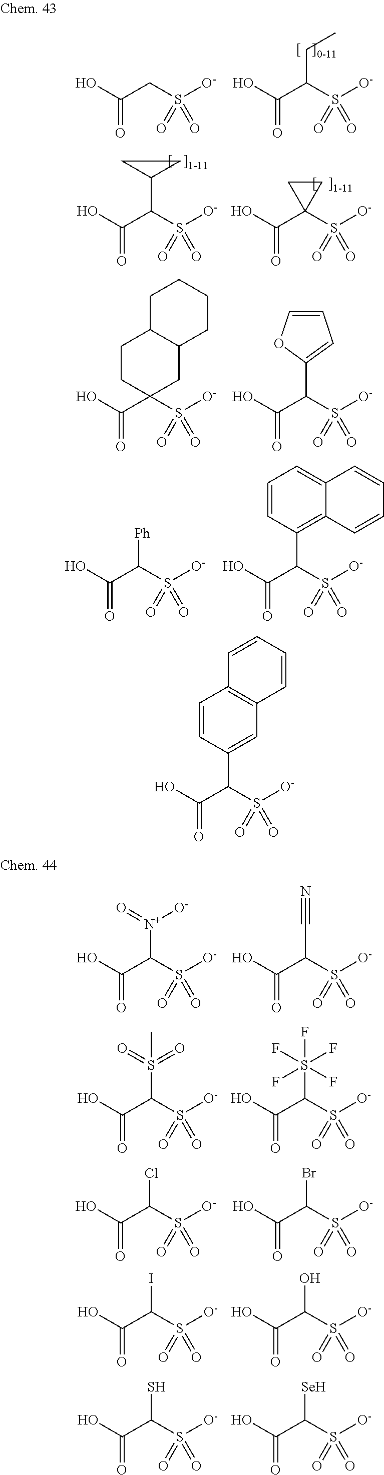

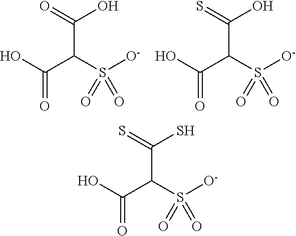

Cation Moiety: (M^{m+})_{1/m}

In the general formula (b1-1), M^{m+} represents an m-valent organic cation.

The organic cation in M^{m+} is preferably an onium cation, more preferably a sulfonium cation, an iodonium cation, or an ammonium cation, and still more preferably a sulfonium cation or an iodonium cation. m is an integer of 1 or more.

Preferred examples of the cation moiety ((M^{m+}) 1/m) include an organic cation represented by any of the following general formulae (ca-1) to (ca-3).

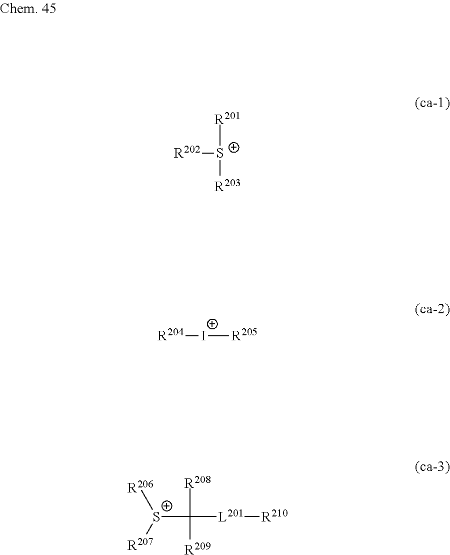

In the general formulae (ca-1) to (ca-3), R²⁰¹ to R²⁰⁷ each independently represent an aryl group that may include a substituent, an alkyl group that may include a substituent, or an alkenyl group that may include a substituent, and R²⁰¹ to R²⁰³ and R²⁰⁶ to R²⁰⁷ may be bonded to each other to form a ring together with the sulfur atom in the formula. R²⁰⁸ and R²⁰⁹ each independently represent a hydrogen atom or an alkyl group having 1 to 5 carbon atoms, R²¹⁰ represents an aryl group that may include a substituent, an alkyl group that may include a substituent, an alkenyl group that may include a substituent, or a —SO₂-containing cyclic group that may include a substituent, and L²⁰¹ represents —C(═O)— or —C(═O)—O—.

Examples of the aryl group in R²⁰¹ to R²⁰⁷ include an unreplaced aryl group having 6 to 20 carbon atoms, and a phenyl group and a naphthyl group are preferred.

The alkyl group in R²⁰¹ to R²⁰⁷ is preferably a chain or cyclic alkyl group having 1 to 30 carbon atoms.

The alkenyl group in R²⁰¹ to R²⁰⁷ preferably has 2 to 10 carbon atoms.

Examples of the substituent that R²⁰¹ to R²⁰⁷ may have include an alkyl group, a halogen atom, a halogenated alkyl group, a carbonyl group, a cyano group, an amino group, an aryl group, an arylthio group, and a group represented by any one of the following formulae (ca-r-1) to (ca-r-7).

Examples of the aryl group in the arylthio group as the substituent include an aryl group having 6 to 20 carbon atoms, and a phenyl group, a naphthyl group, and a biphenyl group are preferred. Examples of the arylthio group include a phenylthio group, a naphthylthio group, and a biphenylthio group.

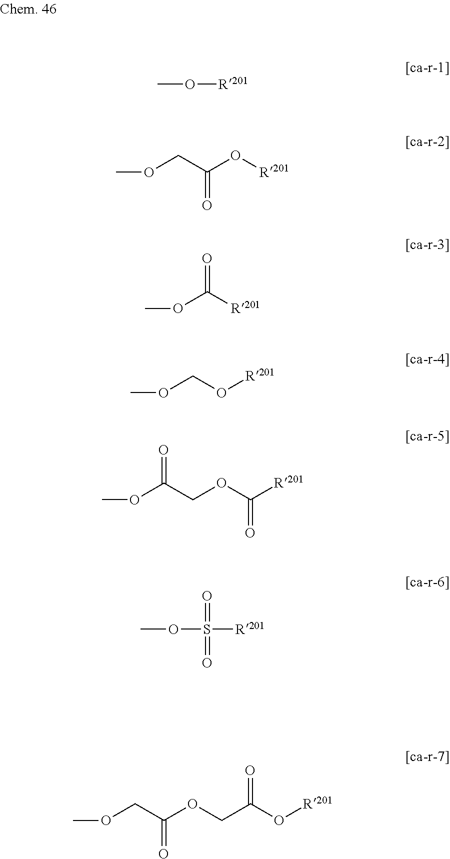

In the formulae, R′²⁰¹'s each independently represent a hydrogen atom, a cyclic group that may include a substituent, a chained alkyl group that may include a substituent, or a chained alkenyl group that may include a substituent.

The cyclic group that may include a substituent and is represented by R′²⁰¹ is preferably a cyclic hydrocarbon group, and the cyclic hydrocarbon group may be an aromatic hydrocarbon group or an aliphatic hydrocarbon group.

Examples of the aromatic hydrocarbon group include an aryl group in which one hydrogen atom is removed from an aromatic hydrocarbon ring or an aromatic compound containing two or more aromatic rings, and a phenyl group and a naphthyl group are preferred.

Examples of the aliphatic hydrocarbon group include a group in which one hydrogen atom is removed from a monocycloalkane or a polycycloalkane, and an adamantyl group and a norbornyl group are preferred.

The chained alkyl group that may include a substituent and is represented by R′²⁰¹ may be either linear or branched.

The linear alkyl group preferably has 1 to 20 carbon atoms, more preferably has 1 to 15 carbon atoms, and most preferably has 1 to 10 carbon atoms. Specific examples thereof include a methyl group, an ethyl group, a propyl group, a butyl group, a pentyl group, a hexyl group, a heptyl group, an octyl group, a nonyl group, a decyl group, an undecyl group, a dodecyl group, a tridecyl group, an isotridecyl group, a tetradecyl group, a pentadecyl group, a hexadecyl group, an isohexadecyl group, a heptadecyl group, an octadecyl group, a nondecyl group, an icosyl group, a henicosyl group, and a docosyl group.

The chained alkenyl group that may include a substituent and is represented by R′²⁰¹ may be either linear or branched, and preferably has 2 to 10 carbon atoms, more preferably has 2 to 5 carbon atoms, still more preferably has 2 to 4 carbon atoms, and particularly preferably has 3 carbon atoms. Examples of the linear alkenyl group include a vinyl group, a propenyl group (an allyl group), and a butenyl group. Examples of the branched alkenyl group include a 1-methylpropenyl group and a 2-methylpropenyl group.

Among them, the chained alkenyl group is particularly preferably a propenyl group.

Examples of the cyclic group that may include a substituent and is represented by R′²⁰¹ or the chained alkyl group that may include a substituent and is represented by R′²⁰¹ include those same as the acid-dissociable group represented by the formula (a1-r2-1).

Examples of the substituent in the cyclic group, the chained alkyl group, or the chain alkenyl group which are represented by R′²⁰¹ include an alkyl group, an alkoxy group, a halogen atom, a halogenated alkyl group, a hydroxy group, a carbonyl group, and a nitro group.

The alkyl group as the substituent is preferably an alkyl group having 1 to 5 carbon atoms, and most preferably a methyl group, an ethyl group, a propyl group, an n-butyl group, or a tert-butyl group.

The alkoxy group as the substituent is preferably an alkoxy group having 1 to 5 carbon atoms, more preferably a methoxy group, an ethoxy group, an n-propoxy group, an iso-propoxy group, an n-butoxy group, or a tert-butoxy group, and most preferably a methoxy group or an ethoxy group.

Examples of the halogen atom as the substituent include a fluorine atom, a chlorine atom, a bromine atom, and an iodine atom. A fluorine atom is preferred.

Examples of the halogenated alkyl group as the substituent include an alkyl group having 1 to 5 carbon atoms, for example, a group in which a part or all of hydrogen atoms of a methyl group, an ethyl group, a propyl group, an n-butyl group, a tert-butyl group, or the like are replaced by halogen atoms.

When R²⁰¹ to R²⁰³ and R²⁰⁶ to R²⁰⁷ are bonded to each other to form a ring together with the sulfur atom in the formula, R²⁰¹ to R²⁰³ and R²⁰⁶ to R²⁰⁷ may be bonded via a heteroatom such as a sulfur atom, an oxygen atom or a nitrogen atom, or a functional group such as a carbonyl group, —SO—, —SO₂—, —SO₃—, —COO—, —CONH— or —N(R_{N})— (the R_{N} is an alkyl group having 1 to 5 carbon atoms). Regarding the ring to be formed, one ring containing the sulfur atom of the formula in the ring skeleton is preferably a 3-membered to 10-membered ring, and particularly preferably a 5-membered to 7-membered ring, including the sulfur atom. Specific examples of the ring to be formed include a thiophene ring, a thiazole ring, a benzothiophene ring, a thianthlene ring, a dibenzthiophene ring, a 9H-thioxanthene ring, a thioxanthone ring, a thianthrene ring, a phenoxathiin ring, a tetrahydrothiophenium ring, a tetrahydrothiopyranium ring, and a thioxanium ring.

R²⁰⁸ and R²⁰⁹ each independently represent a hydrogen atom or an alkyl group having 1 to 5 carbon atoms, and are preferably a hydrogen atom or an alkyl group having 1 to 3 carbon atoms. When R²⁰⁸ and R²⁰⁹ each independently represent an alkyl group, they may be bonded to each other to form a ring.

R²¹⁰ represents an aryl group that may include a substituent, an alkyl group that may include a substituent, an alkenyl group that may include a substituent, or a —SO₂-containing cyclic group that may include a substituent.

Examples of the aryl group in R²¹⁰ include an unreplaced aryl group having 6 to 20 carbon atoms, and a phenyl group and a naphthyl group are preferred.

The alkyl group in R²¹⁰ is preferably a chained or cyclic alkyl group having 1 to 30 carbon atoms.

The alkenyl group in R²¹⁰ preferably has 2 to 10 carbon atoms.

In the —SO₂-containing cyclic group that may include a substituent in R²¹⁰, the “—SO₂-containing cyclic group” refers to a cyclic group containing a ring containing —SO₂-in the ring skeleton thereof, and specifically, the —SO₂-containing cyclic group is a cyclic group in which the sulfur atom(S) in —SO₂— forms a part of the ring skeleton of the cyclic group. When a ring containing —SO₂— in the ring skeleton thereof is counted as a first ring, a group having only the ring is referred to as a monocyclic group, and a group further having another ring structure is referred to as a polycyclic group regardless of the structure. The —SO₂-containing cyclic group may be a monocyclic group or a polycyclic group.

The —SO₂-containing cyclic group is particularly preferably a cyclic group containing —O—SO₂— in the ring skeleton thereof, that is, a cyclic group containing a sultone ring in which —O—S— in —O—SO₂-forms a part of the ring skeleton.

The —SO₂-containing cyclic group that may include a substituent in R²¹⁰ is preferably a group represented by the formula (a5-r-1).

The cation represented by the formula (ca-1) is preferably a cation represented by the following formula (b-2).

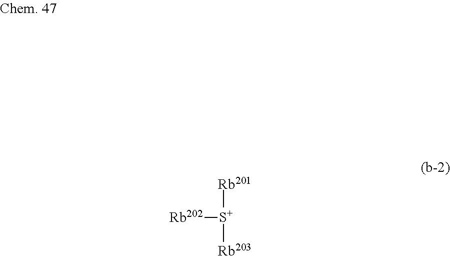

In the formula (b-2), Rb²⁰¹ and Rb²⁰² each represent an aryl group which may have a substituent, and Rb²⁰³ represents an aryl group which may have a substituent, an alkyl group which may have a substituent, or an alkenyl group which may have a substituent. Rb²⁰¹ to Rb²⁰³ may be bonded to each other to form a ring together with a sulfur atom in the formula (b-2).

The aryl group that may include a substituent and is represented by any one of Rb²⁰¹ and Rb²⁰² has the same meaning as the aryl group that may include a substituent as any one of R²⁰¹ to R²⁰⁷, and preferred examples of the aryl group of Rb²⁰¹ and Rb²⁰² are the same as those of the aryl group of R²⁰¹ to R²⁰⁷

The aryl group that may include a substituent, the alkyl group that may include a substituent, or the alkenyl group that may include a substituent that are represented by Rb²⁰³ has the same meaning as the aryl group that may include a substituent, the alkyl group that may include a substituent, or the alkenyl group that may include a substituent as any one of R²⁰¹ to R²⁰⁷, and preferred examples of the groups of Rb²⁰³ are the same as those of the groups of R²⁰¹ to R²⁰⁷.

Specific examples of the suitable cation represented by the formula (ca-1) include a cation represented by any one of the following formulae (ca-1-1) to (ca-1-67).

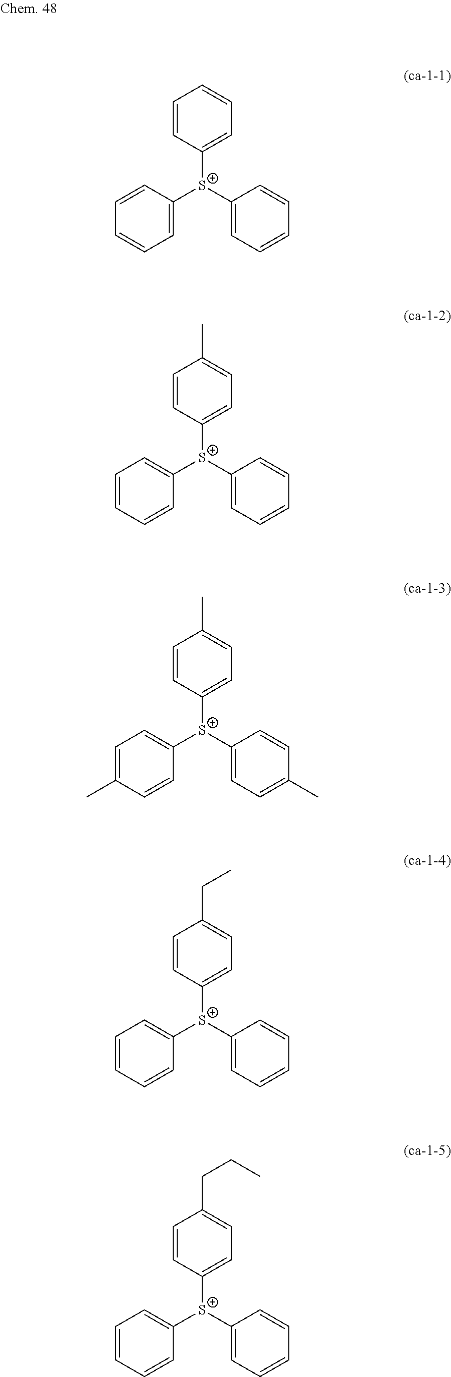

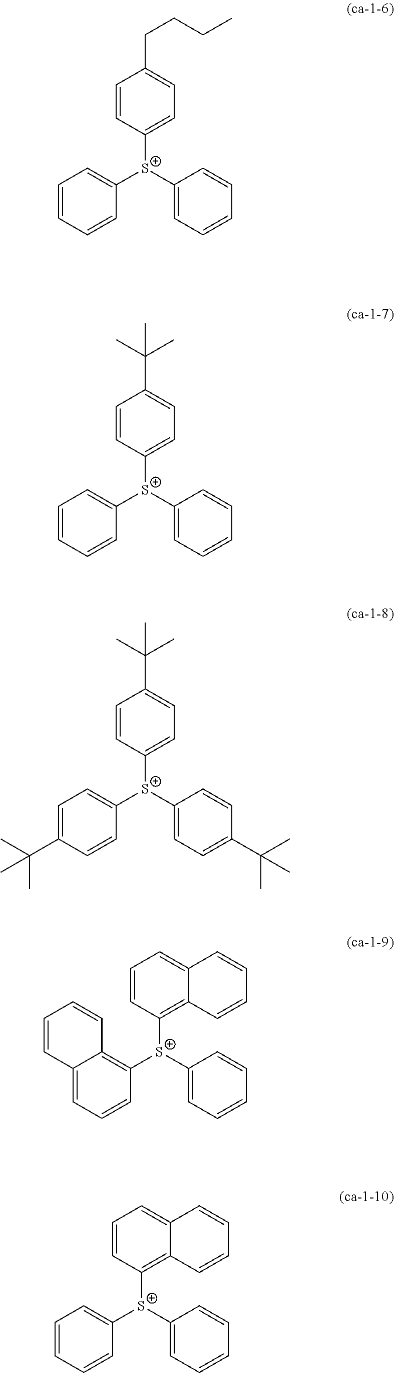

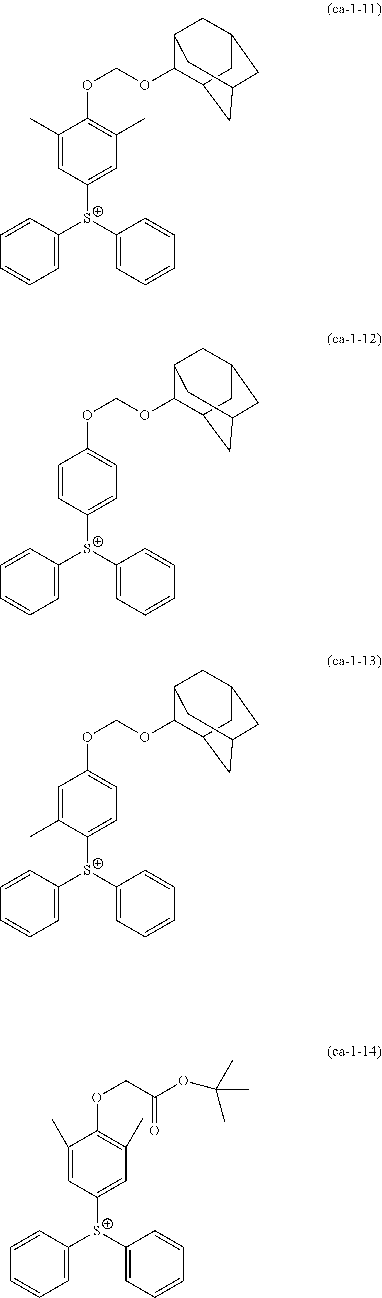

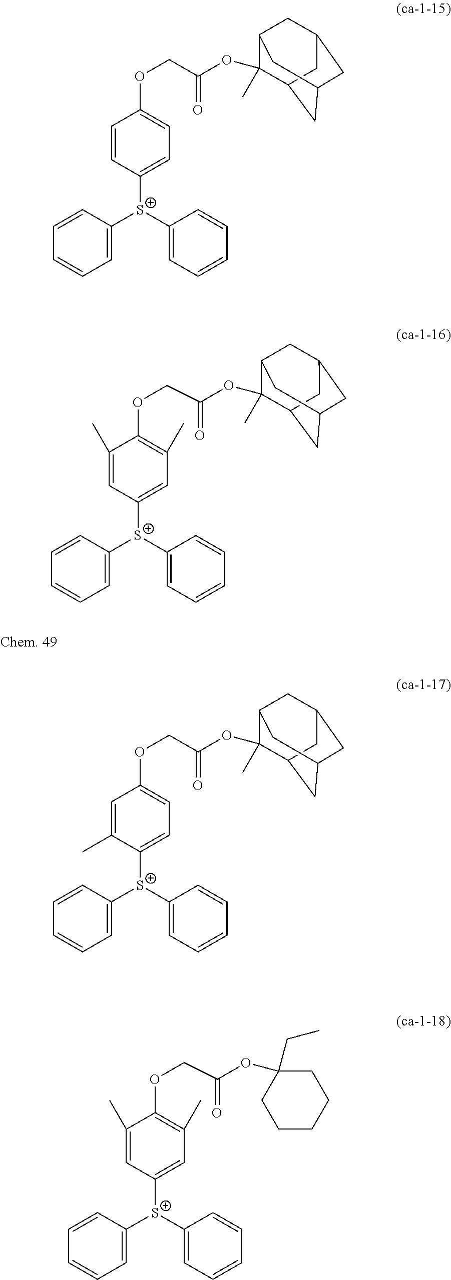

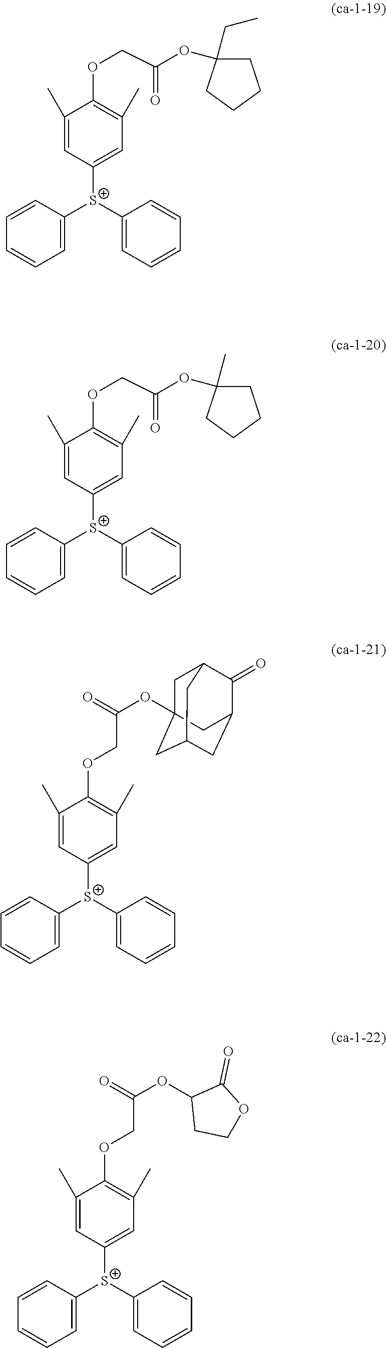

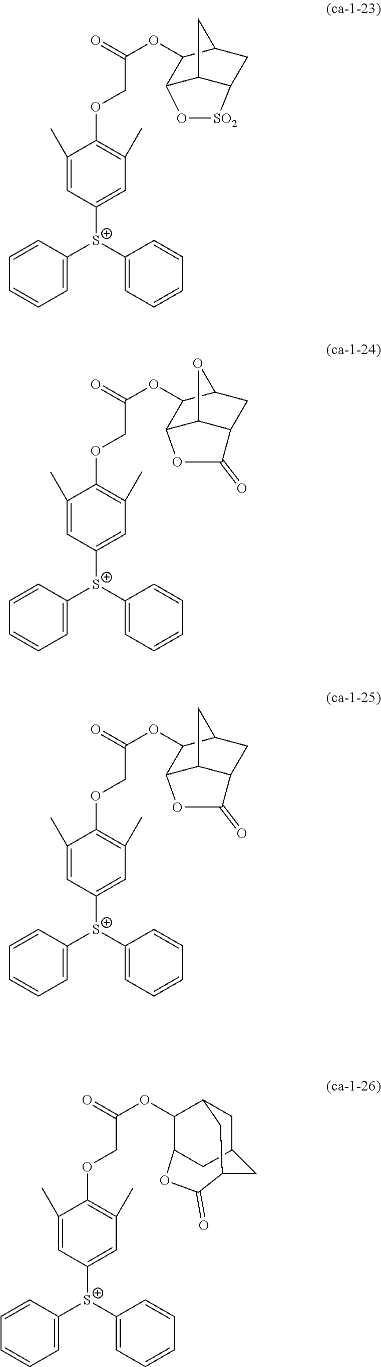

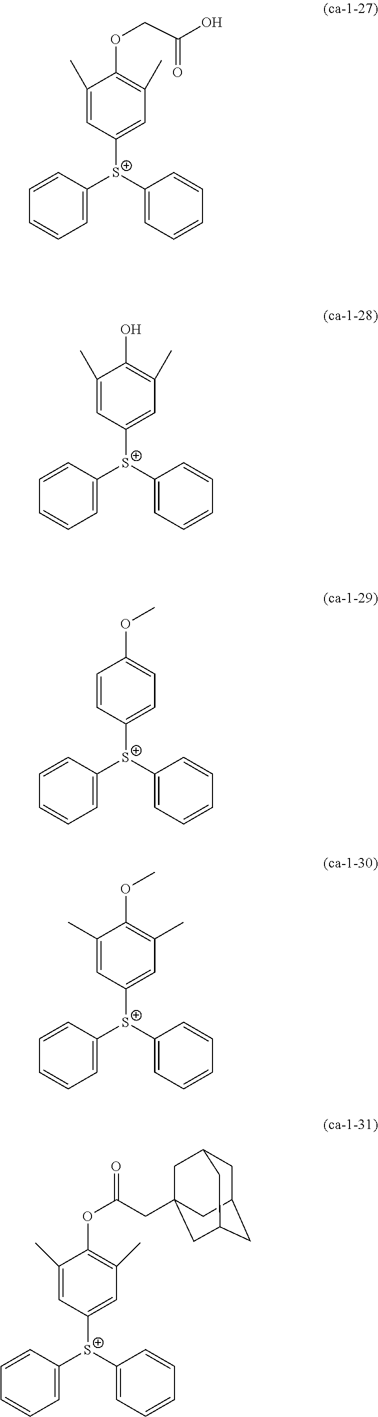

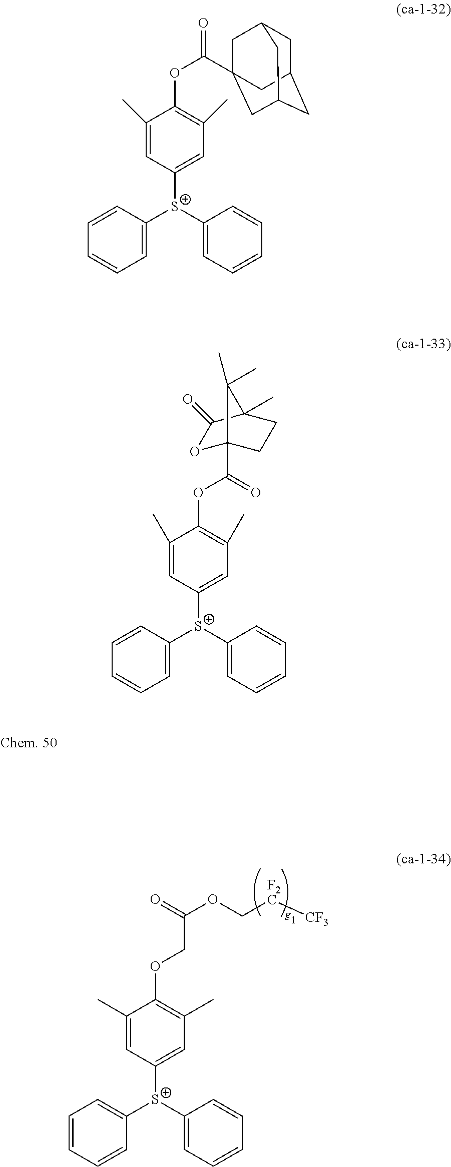

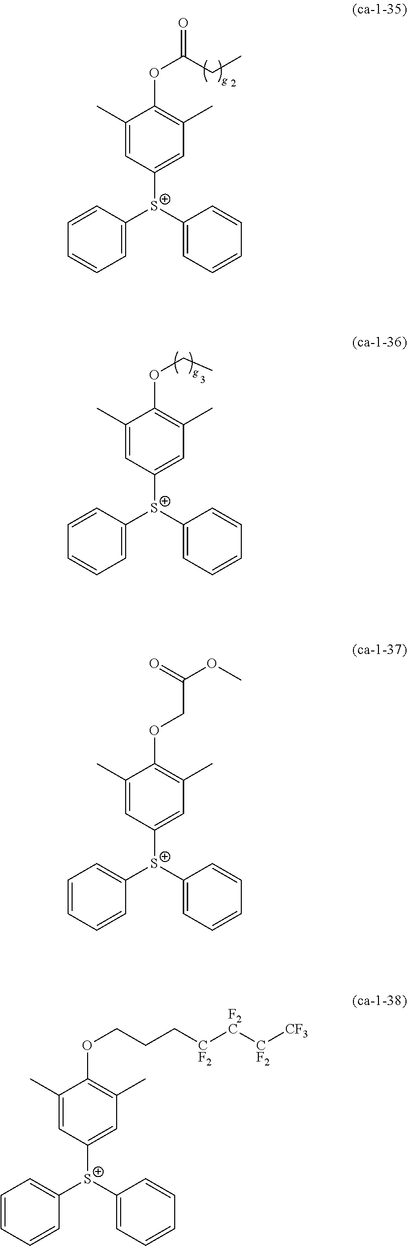

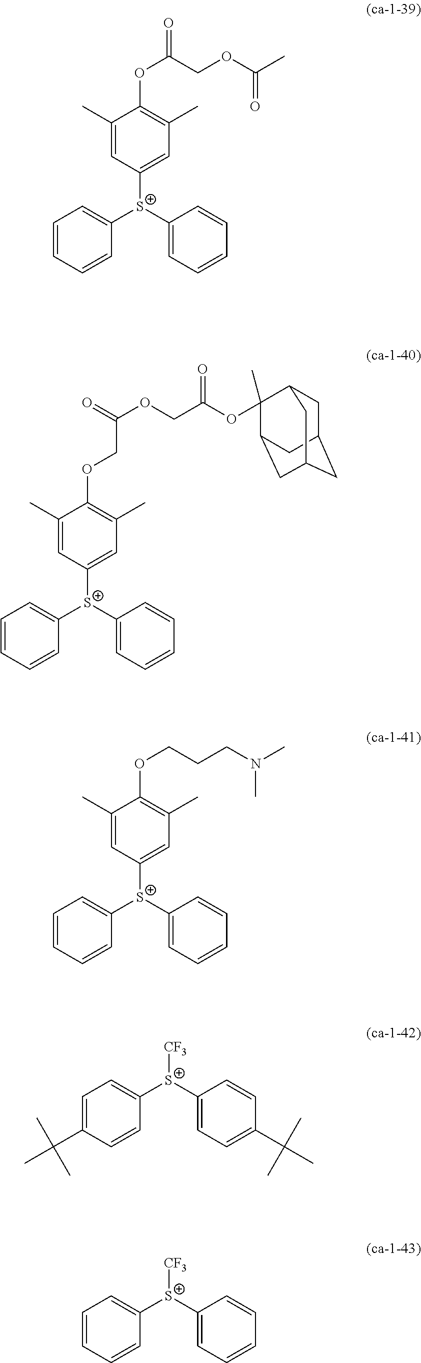

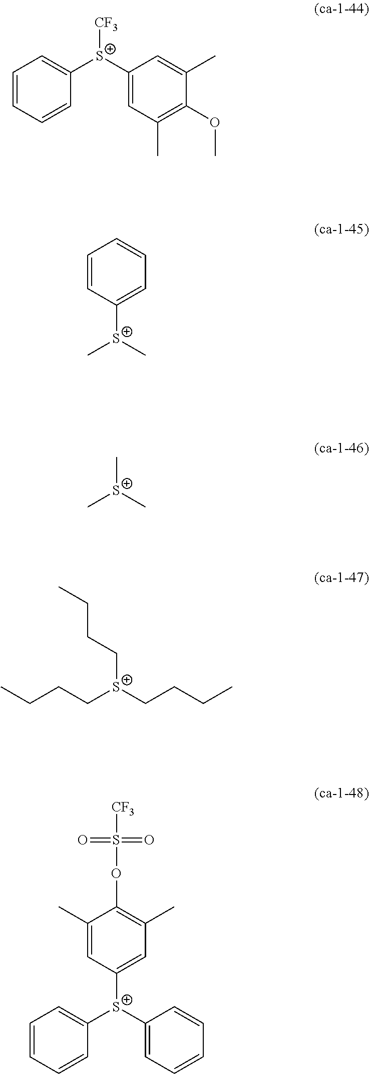

In the formulae, g1, g2, and g3 represent the number of repetitions, g1 is an integer of 1 to 5, g2 is an integer of 0 to 20, and g3 is an integer of 1 to 20.

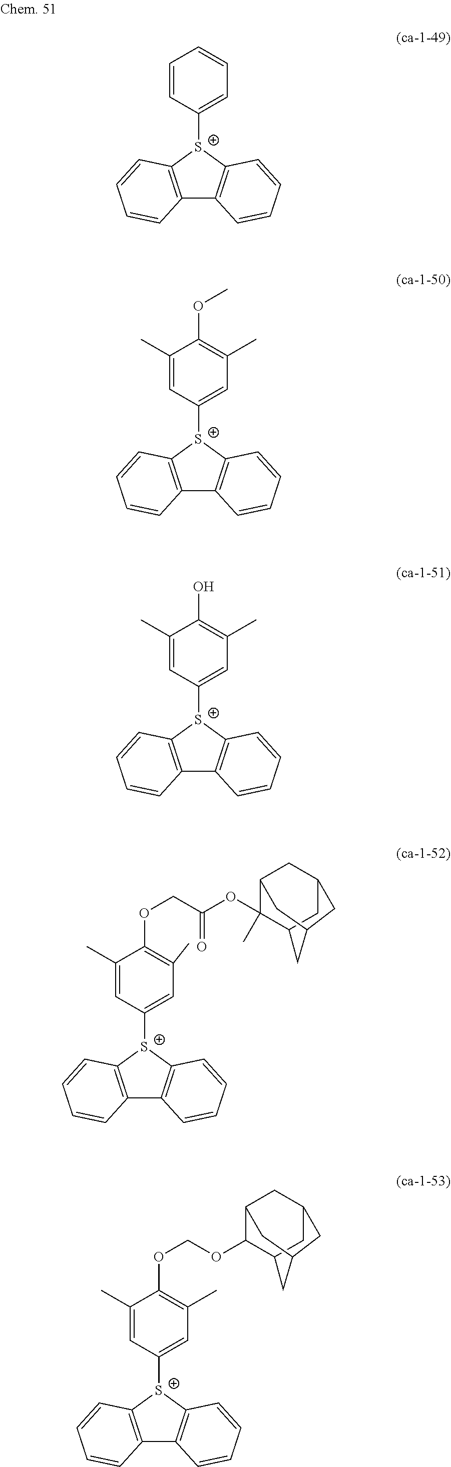

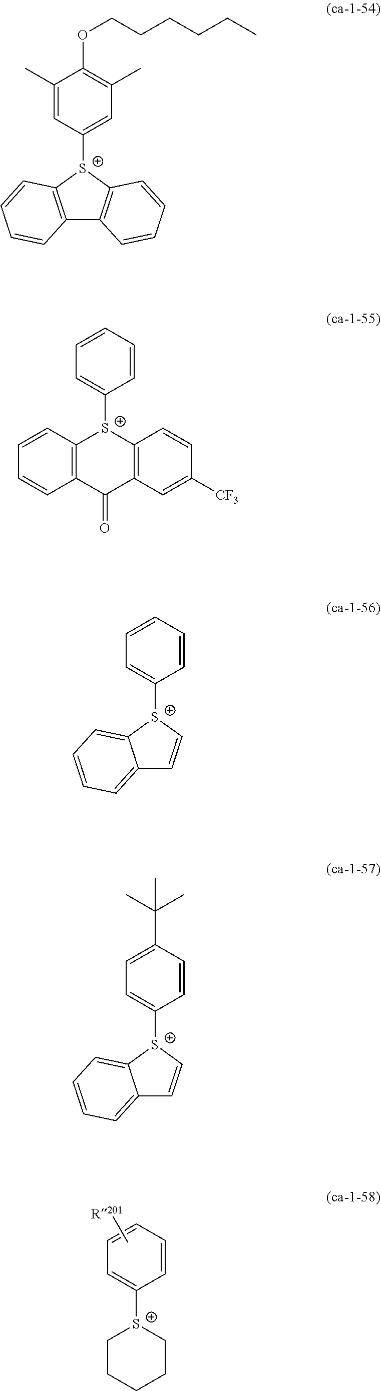

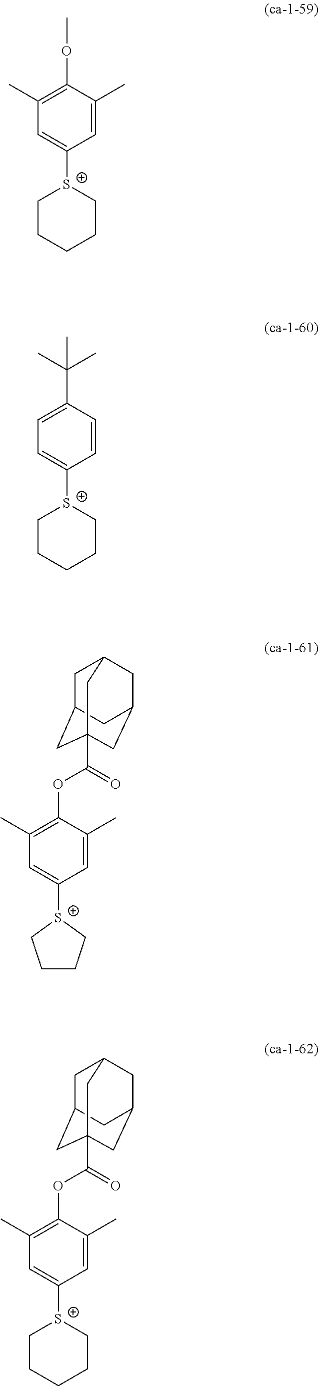

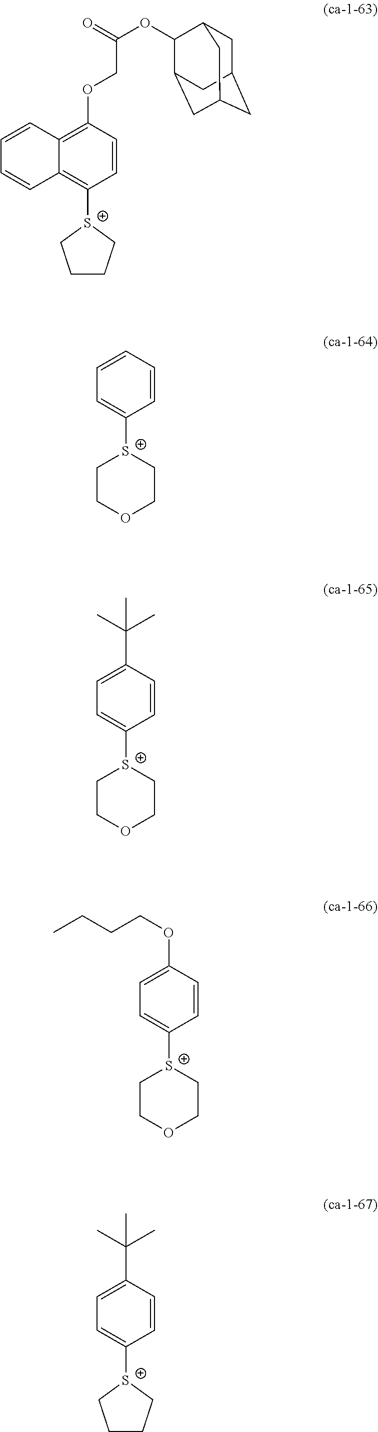

In the formulae, R″²⁰¹ represents a hydrogen atom or a substituent, and the substituent is the same as the substituent that R²⁰¹ to R²⁰⁷, and R²¹⁰ may have.

Specific examples of the suitable cation represented by the formula (ca-3) include a cation represented by any one of the following formulae (ca-3-1) to (ca-3-6).

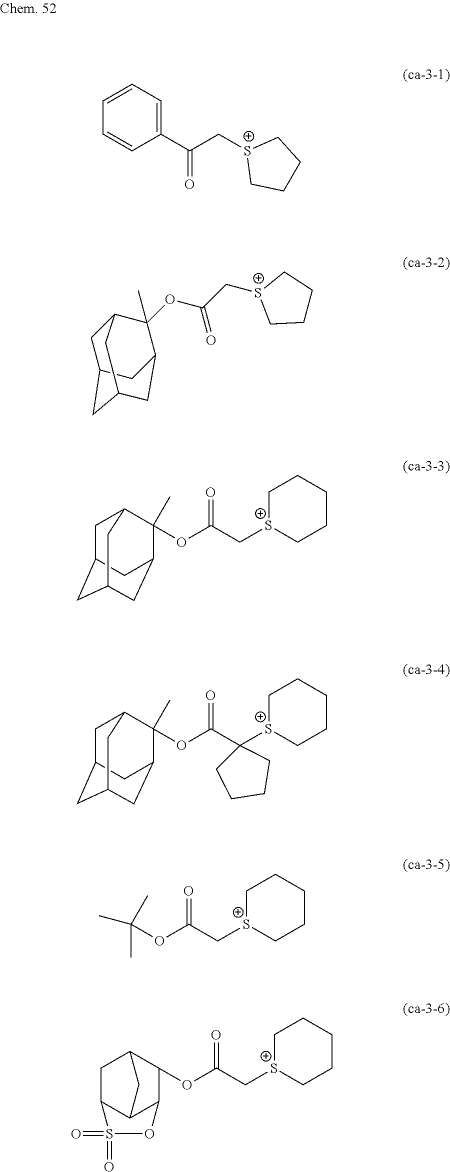 [SMILES: O=C(C[S+]1CCCC1)c1ccccc1 ; CC1(OC(=O)C[S+]2CCCC2)C2CC3CC(C2)CC1C3 ; CC1(OC(=O)C[S+]2CCCCC2)C2CC3CC(C2)CC1C3 ; CC1(OC(=O)C2([S+]3CCCCC3)CCCC2)C2CC3CC(C2)CC1C3 ; CC(C)(C)OC(=O)C[S+]1CCCCC1 ; O=C(C[S+]1CCCCC1)OC1C2CC3C1OS(=O)(=O)C3C2]

In the present embodiment, the compound represented by the general formula (b1-1) is preferably a compound represented by the following general formula (b1-2).

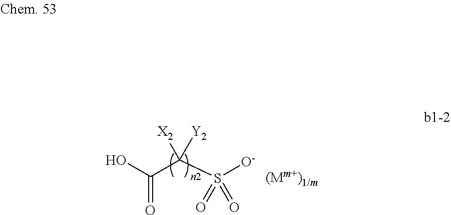

In the general formula (b1-2), X₂ and Y₂ each independently represent a hydrogen atom, an aliphatic hydrocarbon group, or an aromatic hydrocarbon group. n2 represents an integer of 1 or 2. m is an integer of 1 or more. M^{m+} represents an m-valent organic cation.

In the general formula (b1-2), examples of the aliphatic hydrocarbon group and aromatic hydrocarbon group represented by X₂ and Y₂ are the same as the aliphatic hydrocarbon group and aromatic hydrocarbon group represented by X₁ and Y₁ in the general formula (b1-1), respectively.

M^{m+} is an m-valent organic cation, and is the same as M^{m+} in the above formula (b1-1).

In the present embodiment, the compounds represented by the general formulae (b1-1) and (b1-2) are preferably a compound represented by the following general formula (b1-3).

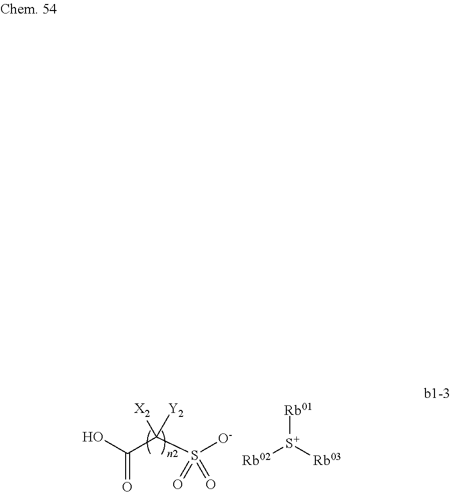

In the general formula (b1-3), X₂ and Y₂ each independently represent a hydrogen atom, an aliphatic hydrocarbon group, or an aromatic hydrocarbon group. n2 represents an integer of 1 or 2. Rb⁰¹ to Rb⁰³ each independently represent an aryl group, an alkyl group, or an alkenyl group. The aryl group, alkyl group, and alkenyl group may each independently have one or more substituents selected from the group consisting of an alkyl group, an aldehyde group, an acyl group, a hydroxyl group, and a halogen atom as a substituent. Rb⁰¹ to Rb⁰³ may be bonded to each other to form a ring together with a sulfur atom in the general formula (b1-3).

In the general formula (b1-3), X₂ and Y₂ are the same as X₂ and Y₂ in the general formula (b1-2), respectively.

The aryl group, alkyl group, or alkenyl group in Rb⁰¹ to Rb⁰³ and the substituent which these may have are the same as the aryl group, alkyl group, or alkenyl group in Rb²⁰¹ to Rb²⁰³ in the general formula (ca-1) and the substituent which these may have, respectively.

Specific examples of the suitable component (B1) are shown below.

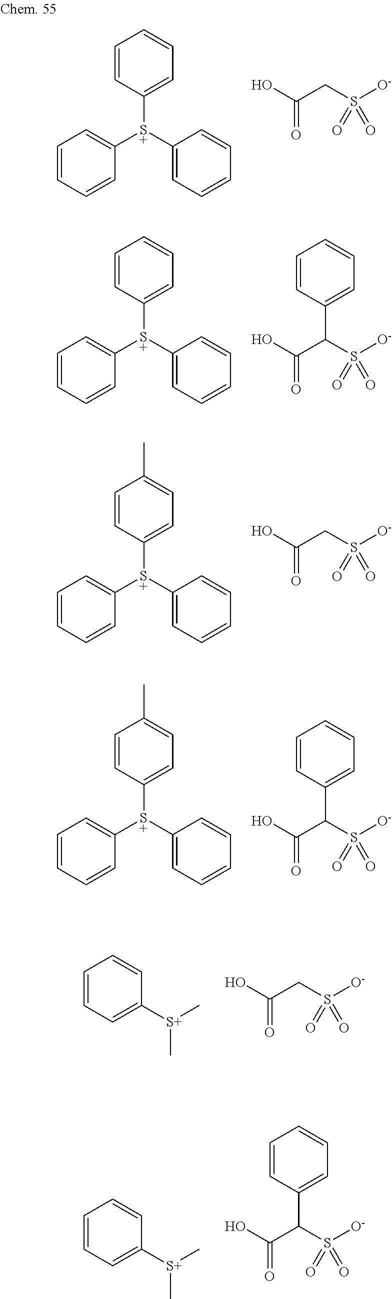

In the resist composition of the present embodiment, the component (B1) may be used alone or in combination of two or more kinds thereof.

In the resist composition of the present embodiment, a content of the component (B1) is preferably 1 part by mass to 40 parts by mass, more preferably 1 part by mass to 30 parts by mass, and still more preferably 1 part by mass to 25 parts by mass, with respect to 100 parts by mass of the component (A).

When the content of the component (B1) is equal to or more than the lower limit value of the above preferred range, lithography properties such as a sensitivity and CDU are further improved in the formation of a resist pattern. On the other hand, when the content of the component (B1) is equal to or less than the upper limit value of the above preferred range, a uniform solution is easily obtained when each component of the resist composition is dissolved in an organic solvent, and the storage stability of the resist composition is further improved.

### Regarding Component (B2)

The resist composition of the present embodiment may contain an acid generating agent component (hereinafter, referred to as “component (B2)”) other than the component (B1) as long as the effects of the present invention are not impaired.

The component (B2) is not particularly limited, and those proposed as an acid generating agent for a chemically amplified resist composition can be used. The component (B2) preferably does not contain a fluorine atom.

Examples of such an acid generating agent include various acid generating agents such as onium salt acid generating agents such as an iodonium salt and a sulfonium salt; oxime sulfonate acid generating agents; diazomethane acid generating agents such as bisalkyl or bisarylsulfonyl diazomethanes and poly(bissulfonyl) diamethanes; nitrobenzyl sulfonate acid generating agents; iminoosulfonate acid generating agents; and disulfone acid generating agents.

In the resist composition of the present embodiment, the component (B2) may be used alone or in combination of two or more kinds thereof.

When the resist composition contains the component (B2), a content of the component (B2) in the resist composition is preferably 50 parts by mass or less, more preferably 1 part by mass to 40 parts by mass, and still more preferably 5 parts by mass to 30 parts by mass, with respect to 100 parts by mass of the component (A).

When the content of the component (B2) is within the range, pattern formation is sufficiently performed. It is preferable that the content of the component (B2) is within the range since a uniform solution is easily obtained when each component of the resist composition is dissolved in an organic solvent, and storage stability of the resist composition is improved.

### <<Component (D)>>

The resist composition in the present embodiment may further contain an acid diffusion control agent component (hereinafter, referred to as “component (D)”) in addition to the component (A) and the component (B). The component (D) acts as a quencher that traps acid generated in the resist composition by exposure.

The component (D) may be a photodegradable base (D1) (hereinafter, referred to as “component (D1)”) which is decomposed by exposure and loses acid diffusion controllability, or may be a nitrogen-containing organic compound (D2) (hereinafter, referred to as “component (D2)”) not corresponding to the component (D1), and is preferably the component (D1).

By using a resist composition containing the component (D), a contrast between an exposed portion and an unexposed portion of the resist film can be further improved when forming a resist pattern.

The component (D) does not contain any compound that falls under the PFAS. The component (D) contains only compounds that do not fall under the PFAS. That is, the component (D) does not contain any compound having a trifluoromethyl group or any compound having a difluoromethylene group. Further, it is preferable that the component (D) do not contain fluorine atoms.

### Regarding Component (D1)

When the resist composition contains the component (D1), a contrast between an exposed portion and an unexposed portion of the resist film can be further improved when forming a resist pattern.

The component (D1) may be used as an acid generating agent in addition to the component (B) or in place of the component (B).

The component (D1) is not particularly limited as long as it is decomposed by exposure and loses acid diffusion controllability, and is preferably one or more compounds selected from the group consisting of a compound represented by the following general formula (d1-1) (hereinafter, referred to as “component (d1-1)”), a compound represented by the following general formula (d1-2) (hereinafter, referred to as “component (d1-2)”), and a compound represented by the following general formula (d1-3) (hereinafter, referred to as “component (d1-3)”). As the components (d1-1) to (d1-3), those corresponding to the general formula (b1-1) are excluded.

The components (d1-1) to (d1-3) do not act as a quencher in an exposed portion of the resist film because they are decomposed and lose acid diffusion controllability (basicity), but act as a quencher in an unexposed portion of the resist film.

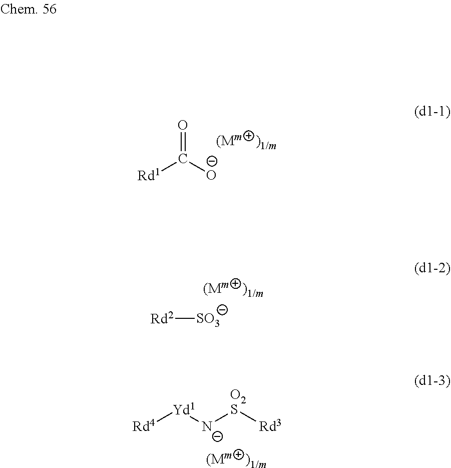

In the formulae, Rd¹ to Rd⁴ represent a cyclic group that may include a substituent, a chained alkyl group that may include a substituent, or a chained alkenyl group that may include a substituent. In Rd² in the formula (d1-2), no fluorine atom is bonded to a carbon atom adjacent to the S atom. Yd¹ represents a single bond or a divalent linking group. m represents an integer of 1 or more. M^{m+}'s each independently represent an m-valent organic cation.

{Component (d1-1)}

### Anion Moiety

In the formula (d1-1), Rd¹ represents a cyclic group that may include a substituent, a chained alkyl group that may include a substituent, or a chained alkenyl group that may include a substituent, each of which is the same as R′201 described above.

Among them, as Rd¹, an aromatic hydrocarbon group that may include a substituent, an aliphatic cyclic group that may include a substituent, and a chained alkyl group that may include a substituent are preferred. Examples of the substituent that the groups may have include a hydroxy group, an oxo group, an alkyl group, an aryl group, a fluorine atom, a fluorinated alkyl group, a lactone-containing cyclic group, an ether bond, an ester bond, or a combination thereof. When an ether bond or an ester bond is contained as the substituent, an alkylene group may be interposed, and as the substituent in this case, a linking group represented any one of by the following formulae (y-a1-1) to (y-a1-8) is preferred.

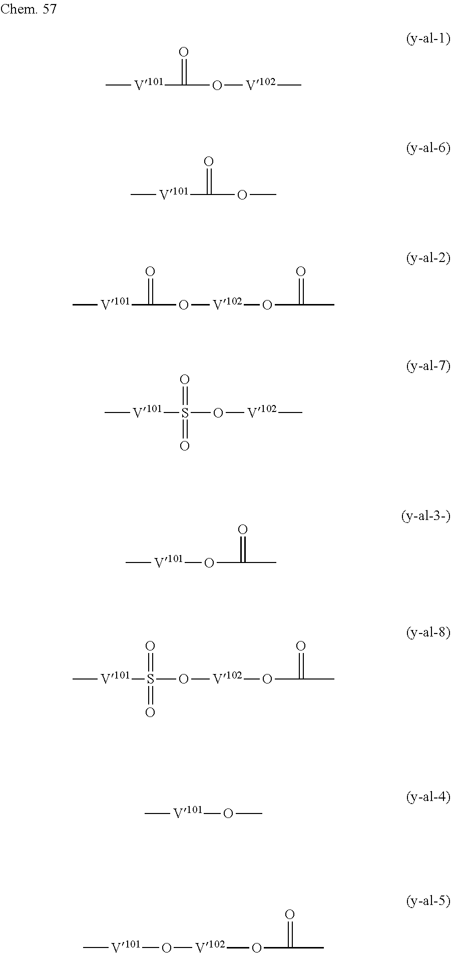

In the formulae, V′¹⁰¹ represents a single bond or an alkylene group having 1 to 5 carbon atoms, and V′¹⁰² represents a divalent saturated hydrocarbon group having 1 to 30 carbon atoms.

In the formulae, the divalent saturated hydrocarbon group represented by V′¹⁰² is preferably an alkylene group having 1 to 30 carbon atoms. The alkylene group in V′¹⁰² is preferably an alkylene group having 1 to 30 carbon atoms, more preferably an alkylene group having 1 to 10 carbon atoms, and still more preferably an alkylene group having 1 to 5 carbon atoms.

Suitable examples of the aromatic hydrocarbon group include a phenyl group, a naphthyl group, and a polycyclic structure containing a bicyclooctane skeleton (a polycyclic structure consisting of a bicyclooctane skeleton and other ring structures).

The aliphatic cyclic group is more preferably a group in which one or more hydrogen atoms are removed from a polycycloalkane such as adamantane, norbornane, isobornane, tricyclo[5.2.1.02,6]decane, or tetracyclododecane.

The chained alkyl group preferably has 1 to 10 carbon atoms. Specific examples thereof include linear alkyl groups such as a methyl group, an ethyl group, a propyl group, a butyl group, a pentyl group, a hexyl group, a heptyl group, an octyl group, a nonyl group, and a decyl group; and branched alkyl groups such as a 1-methylethyl group, a 1-methylpropyl group, a 2-methylpropyl group, a 1-methylbutyl group, a 2-methylbutyl group, a 3-methylbutyl group, a 1-ethylbutyl group, a 2-ethylbutyl group, a 1-methylpentyl group, a 2-methylpentyl group, a 3-methylpentyl group, and a 4-methylpentyl group.

When the chained alkyl group is a fluorinated alkyl group having a fluorine atom or a fluorinated alkyl group as a substituent, the number of carbon atoms of the fluorinated alkyl group is preferably 1 to 11, more preferably 1 to 8, and still more preferably 1 to 4. The fluorinated alkyl group may contain an atom other than a fluorine atom. Examples of the atom other than a fluorine atom include an oxygen atom, a sulfur atom, and a nitrogen atom.

Rd¹ is preferably a fluorinated alkyl group in which a part or all of hydrogen atoms constituting a linear alkyl group are replaced by fluorine atoms, and particularly preferably a fluorinated alkyl group in which all of the hydrogen atoms constituting the linear alkyl group are replaced by fluorine atoms (a linear perfluoroalkyl group).

Specific preferred examples of the anion moiety of the component (d1-1) are shown below.

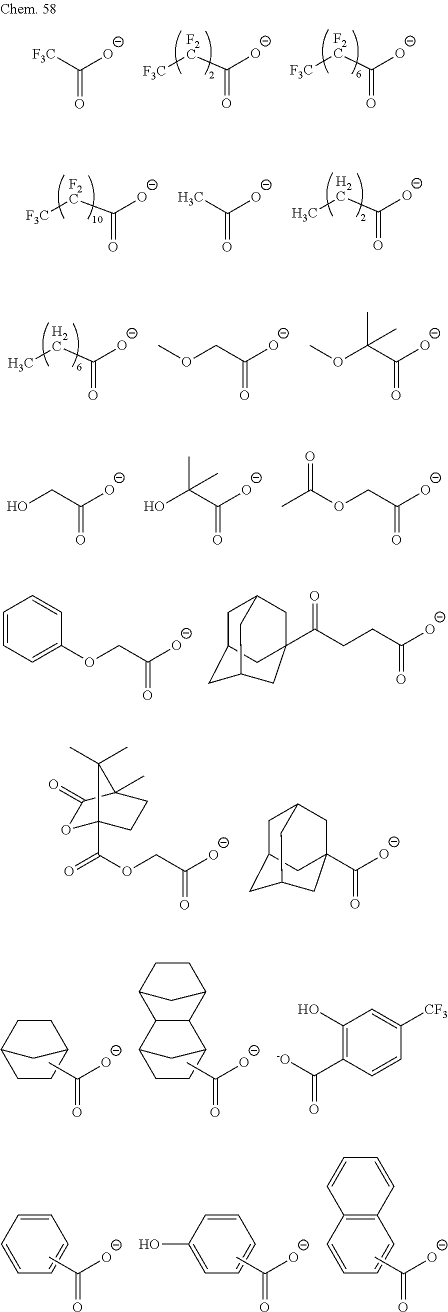

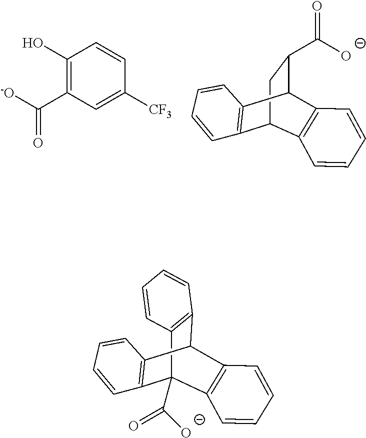

### Cation Moiety

In the formula (d1-1), M^{m+} represents an m-valent organic cation.

Suitable examples of the organic cation of M^{m+} include those same as a cation represented by any one of the general formulae (ca-1) to (ca-3). A cation represented by the general formula (ca-1) is more preferred, and a cation represented by any one of the formulae (ca-1-1) to (ca-1-67) is even more preferred.

The component (d1-1) may be used alone or in combination of two or more kinds thereof.

{Component (d1-2)}

### Anion Moiety

In the formula (d1-2), Rd² represents a cyclic group that may include a substituent, a chained alkyl group that may include a substituent, or a chained alkenyl group that may include a substituent, each of which is the same as R′201 described above.

In Rd², a carbon atom adjacent to the S atom does not have a fluorine atom bonded thereto (is not replaced by fluorine). Accordingly, the anion of the component (d1-2) becomes an appropriate weak acid anion, and a quenching ability of the component (D) is improved.

Rd² is preferably a chained alkyl group that may include a substituent, or an aliphatic cyclic group that may include a substituent. The chained alkyl group preferably has 1 to 10 carbon atoms, and more preferably has 3 to 10 carbon atoms. The aliphatic cyclic group is more preferably a group (that may include a substituent) in which one or more hydrogen atoms are removed from adamantane, norbornane, isobornane, tricyclo[5.2.1.02,6]decane, tetracyclododecane, or the like; or a group in which one or more hydrogen atoms are removed from camphor, or the like.

The hydrocarbon group for Rd² may include a substituent, and examples of the substituent include those same as substituents that the hydrocarbon group (the aromatic hydrocarbon group, the aliphatic cyclic group, or the chained alkyl group) for Rd¹ in the formula (d1-1) may have.

Specific preferred examples of the anion moiety of the component (d1-2) are shown below.

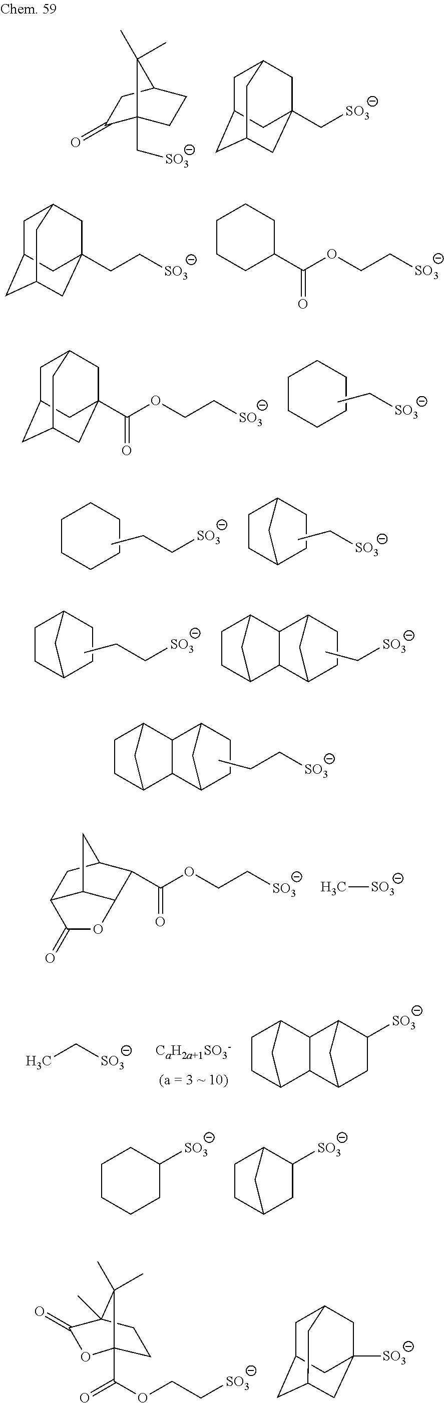

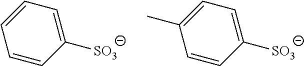

### Cation Moiety

In the formula (d1-2), M^{m+} represents an m-valent organic cation, and is the same as M^{m+} in the formula (d1-1).

The component (d1-2) may be used alone or in combination of two or more kinds thereof.

{Component (d1-3)}

### Anion Moiety

In the formula (d1-3), Rd³ represents a cyclic group that may include a substituent, a chained alkyl group that may include a substituent, or a chained alkenyl group that may include a substituent, each of which is the same as R′²⁰¹ described above. A fluorine atom-containing cyclic group, a chained alkyl group, or a chained alkenyl group is preferred. Among them, a fluorinated alkyl group is preferred, and a fluorinated alkyl group same as those described above for Rd¹ is more preferred.

In the formula (d1-3), Rd⁴ represents a cyclic group that may include a substituent, a chained alkyl group that may include a substituent, or a chained alkenyl group that may include a substituent, each of which is the same as R′²⁰¹ described above.

Among them, an alkyl group, an alkoxy group, an alkenyl group, or a cyclic group, each of which may have a substituent, is preferred.

The alkyl group in Rd⁴ is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group. A part of hydrogen atoms of the alkyl group for Rd⁴ may be replaced by a hydroxy group, a cyano group, or the like.

The alkoxy group in Rd⁴ is preferably an alkoxy group having 1 to 5 carbon atoms, and specific examples of the alkoxy group having 1 to 5 carbon atoms include a methoxy group, an ethoxy group, an n-propoxy group, an iso-propoxy group, an n-butoxy group, and a tert-butoxy group. Among them, a methoxy group and an ethoxy group are preferred.

Examples of the alkenyl group in Rd⁴ include those same as the alkenyl group in R′²⁰¹, and a vinyl group, a propenyl group (an allyl group), a 1-methylpropenyl group, and a 2-methylpropenyl group are preferred. These groups may further have an alkyl group having 1 to 5 carbon atoms or a halogenated alkyl group having 1 to 5 carbon atoms as a substituent.

Examples of the cyclic group in Rd⁴ include those same as the cyclic group in R′²⁰¹. The cyclic group in Rd⁴ is preferably an alicyclic group in which one or more hydrogen atoms are removed from a cycloalkane such as cyclopentane, cyclohexane, adamantane, norbornane, isobornane, tricyclo[5.2.1.02,6]decane, or tetracyclododecane, or an aromatic group such as a phenyl group or a naphthyl group. When Rd⁴ is an alicyclic group, the resist composition has a good solubility in an organic solvent, and thus the lithography properties become excellent. When Rd⁴ is an aromatic group, the resist composition has an excellent light absorption efficiency, a good sensitivity, and good lithography properties in lithography using EUV or the like as a light source for exposure.

In the formula (d1-3), Yd¹ represents a single bond or a divalent linking group.

The divalent linking group in Yd¹ is not particularly limited, and examples thereof include a divalent hydrocarbon group (an aliphatic hydrocarbon group, an aromatic hydrocarbon group) that may include a substituent, and a divalent linking group containing a heteroatom.

Examples of these groups include those same as the divalent hydrocarbon group that may include a substituent and the divalent linking group containing a heteroatom, which are described in the description of the divalent linking group represented by Ya²¹ in the general formula (a2-1).

Yd¹ is preferably a carbonyl group, an ester bond, an amide bond, an alkylene group, or a combination thereof. The alkylene group is more preferably a linear or branched alkylene group, and still more preferably a methylene group or an ethylene group.

Specific preferred examples of the anion moiety of the component (d1-3) are shown below.

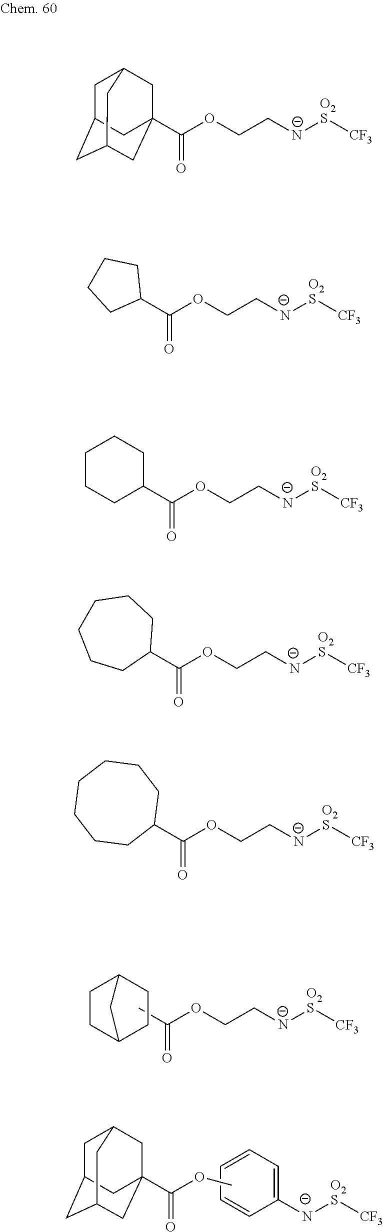

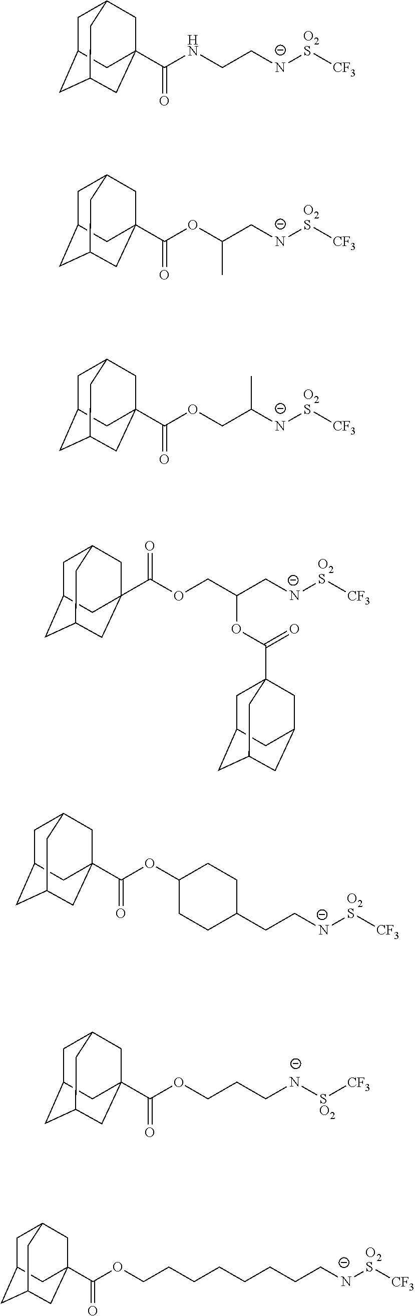

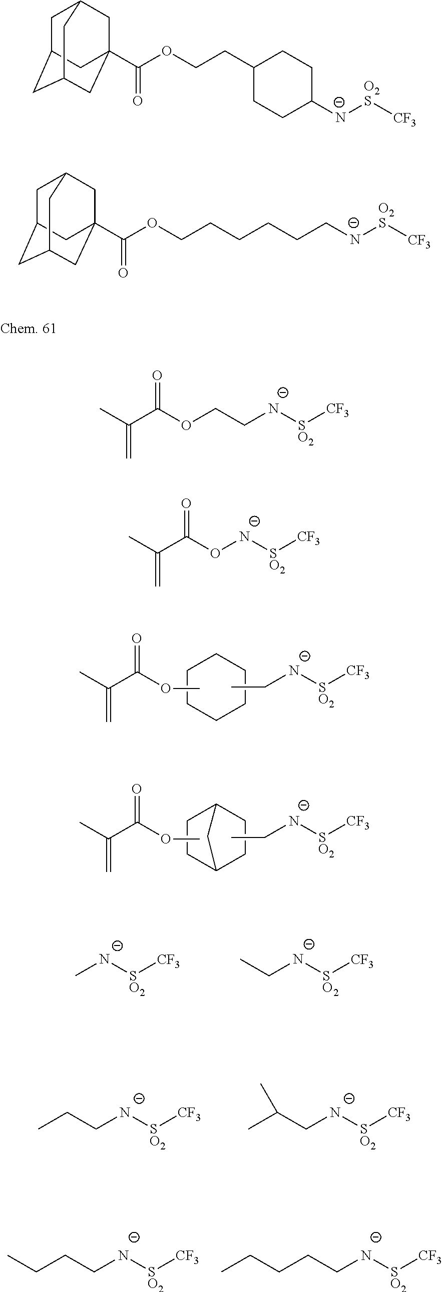

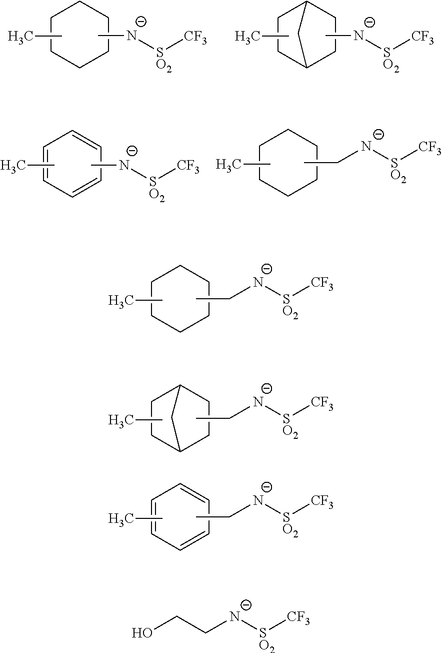

### Cation Moiety

In the formula (d1-3), M^{m+} represents an m-valent organic cation, and is the same as M^{m+} in the formula (d1-1).

The component (d1-3) may be used alone or in combination of two or more kinds thereof.

As the component (D1), only one kind of the components (d1-1) to (d1-3) may be used, or two or more kinds thereof may be used in combination.

When the resist composition contains the component (D1), a content of the component (D1) in the resist composition is preferably 0.5 parts by mass to 25 parts by mass, more preferably 1 part by mass to 20 parts by mass, and still more preferably 2.5 parts by mass to 15 parts by mass, with respect to 100 parts by mass of the component (A).

When the content of the component (D1) is equal to or larger than a preferred lower limit value, particularly good lithography properties and a particularly good resist pattern shape are easily obtained. On the other hand, when the content of the component (D1) is equal to or less than an upper limit value, the sensitivity can be favorably maintained and the throughput is also excellent.

### Method for Producing Component (D1):

A method for producing the component (d1-1) and the component (d1-2) is not particularly limited, and the component (d1-1) and the component (d1-2) can be produced by known methods.

A method for producing the component (d1-3) is not particularly limited, and the component (d1-3) can be produced, for example, in the same manner as a method described in US2012-0149916.

### Regarding Component (D2)

The acid diffusion control agent component may contain a nitrogen-containing organic compound component (hereinafter, referred to as “component (D2)”) not corresponding to the component (D1).

The component (D2) is not particularly limited as long as it acts as an acid diffusion control agent and does not correspond to the component (D1), and any known compounds may be used. Among them, an aliphatic amine or an aromatic amine is preferred, and an aromatic amine is more preferred.

The aliphatic amine is an amine having one or more aliphatic groups, and the aliphatic group preferably has 1 to 12 carbon atoms.

Examples of the aliphatic amine include an amine in which at least one hydrogen atom of ammonia NH₃ is replaced by an alkyl group or a hydroxyalkyl group having 12 or less carbon atoms (an alkyl amine or an alkyl alcohol amine), and a cyclic amine.

Specific examples of the alkyl amine and the alkyl alcohol amine include monoalkylamines such as n-hexylamine, n-heptylamine, n-octylamine, n-nonylamine, and n-decylamine; dialkylamines such as diethylamine, di-n-propylamine, di-n-heptylamine, di-n-octylamine, and dicyclohexylamine; trialkylamines such as trimethylamine, triethylamine, tri-n-propylamine, tri-n-butylamine, tri-n-pentylamine, tri-n-hexylamine, tri-n-heptylamine, tri-n-octylamine, tri-n-nonylamine, tri-n-decylamine, and tri-n-dodecylamine; and alkyl alcohol amines such as diethanolamine, triethanolamine, diisopropanolamine, triisopropanolamine, di-n-octanolamine, and tri-n-octanolamine. Among them, trialkylamine having 6 to 30 carbon atoms is more preferred, and tri-n-pentyl amine or tri-n-octyl amine is particularly preferred.

Examples of the cyclic amine include a heterocyclic compound containing a nitrogen atom as a heteroatom. The heterocyclic compound may be a monocyclic compound (an aliphatic monocyclic amine) or a polycyclic compound (an aliphatic polycyclic amine).

Specific examples of the aliphatic monocyclic amine include piperidine and piperazine.

The aliphatic polycyclic amine preferably has 6 to 10 carbon atoms, and specific examples thereof include 1,5-diazabicyclo[4.3.0]-5-nonene, 1,8-diazabicyclo[5.4.0]-7-undecene, hexamethylenetetramine, and 1,4-diazabicyclo[2.2.2]octane.

Examples of other aliphatic amines include tris(2-methoxymethoxyethyl)amine, tris{2-(2-methoxyethoxy)ethyl}amine, tris{2-(2-methoxyethoxymethoxy)ethyl}amine, tris{2-(1-methoxyethoxy)ethyl}amine, tris{2-(1-ethoxyethoxy)ethyl}amine, tris{2-(1-ethoxypropoxy)ethyl}amine, tris[2-{2-(2-hydroxyethoxy)ethoxy}ethyl]amine, and triethanolamine triacetate. Triethanolamine triacetate is preferred.

As the component (D2), an aromatic amine may be used.

Examples of the aromatic amine include 4-dimethylaminopyridine, 2,6-di-tert-butylpyridine, pyrrole, indole, pyrazole, imidazole or derivatives thereof, tribenzylamine, 2,6-diisopropylaniline, and N-tert-butoxycarbonylpyrrolidine.

The component (D2) may be used alone or in combination of two or more kinds thereof.

When the resist composition contains the component (D2), a content of the component (D2) in the resist composition is generally in a range of 0.01 parts by mass to 5 parts by mass with respect to 100 parts by mass of the component (A). When the content is within the range, the resist pattern shape and the post-exposure temporal stability are improved.

### <Optional Component>

The resist composition of the present embodiment may further contain a component (an optional component) other than the component (A) and component (B).

Examples of the optional component include the following (E) component, (F) component, (H) component, and(S) component.

It is preferred that the component (E), the component (F), the component (H), and the component(S) do not contain any compound that falls under the PFAS. The component (E), the component (F), the component (H), and the component(S) preferably contain neither a compound having a trifluoromethyl group nor a compound having a difluoromethylene group. Further, it is more preferable that no fluorine atom be contained.

<<Component (E): At Least One Compound Selected from Group Consisting of Organic Carboxylic Acid, and Phosphorus Oxoacid and Derivative Thereof>>

The resist composition of the present embodiment may contain at least one compound (E) (hereinafter, referred to as “component (E)”) selected from the group consisting of an organic carboxylic acid, and a phosphorus oxoacid and a derivative thereof as an optional component for the purpose of preventing deterioration in sensitivity, and improving the shape of a resist pattern, the post-exposure temporal stability, and the like.

Suitable examples of the organic carboxylic acid include acetic acid, malonic acid, citric acid, malic acid, succinic acid, benzoic acid, hydroxybenzoic acid, salicylic acid, phthalic acid, terephthalic acid, and isophthalic acid.

Examples of the phosphorus oxoacid include phosphoric acid, phosphonic acid, and phosphinic acid. Among them, phosphonic acid is particularly preferred.

Examples of the derivative of phosphorus oxoacid include an ester in which a hydrogen atom of an oxo acid is replaced by a hydrocarbon group, and examples of the hydrocarbon group include an alkyl group having 1 to 5 carbon atoms and an aryl group having 6 to 15 carbon atoms.

Examples of the derivative of phosphoric acid include phosphate esters such as di-n-butyl phosphate and diphenyl phosphate.

Examples of the derivative of phosphonic acid include phosphonate esters such as dimethyl phosphonate, di-n-butyl phosphonate, phenyl phosphonic acid, diphenyl phosphonate, and dibenzyl phosphonate.

Examples of the derivative of phosphinic acid include phosphinate esters and phenylphosphinic acid.

Among them, the component (E) is preferably an organic carboxylic acid, and more preferably an aromatic carboxylic acid. Specifically, benzoic acid, hydroxybenzoic acid, salicylic acid, phthalic acid, terephthalic acid, and isophthalic acid are preferred. Among them, salicylic acid is more preferred.

In the resist composition of the present embodiment, the component (E) may be used alone or in combination of two or more kinds thereof.

When the resist composition contains the component (E), a content of the component (E) is preferably 0.01 parts by mass to 5 parts by mass, more preferably 0.1 parts by mass to 5 parts by mass, and still more preferably 0.1 parts by mass to 3 parts by mass, with respect to 100 parts by mass of the component (A).

### <<Component (F): Fluorine Additive Component>>

The resist composition of the present embodiment may contain a fluorine additive component (hereinafter, referred to as “component (F)”) as a hydrophobic resin. The component (F) is used to impart water-repellent to the resist film. When the component (F) is used as a resin different from the component (A), the lithography properties are improved.

As the component (F), for example, fluorinated macromolecular compounds described in JP2010-002870A, JP2010-032994A, JP2010-277043A, JP2011-13569A, and JP2011-128226A can be used.

More specific examples of the component (F) include a polymer having a structural unit (f11) represented by the following general formula (f1-1).

The polymer having a structural unit (f11) represented by the following general formula (f1-1) is preferably a polymer (homopolymer) consisting only of a structural unit (f11) represented by the following general formula (f1-1); a copolymer of the structural unit (f11) and the structural unit (a3); or a copolymer of the structural unit (f11), a structural unit derived from acrylic acid or methacrylic acid, and the structural unit (a1).

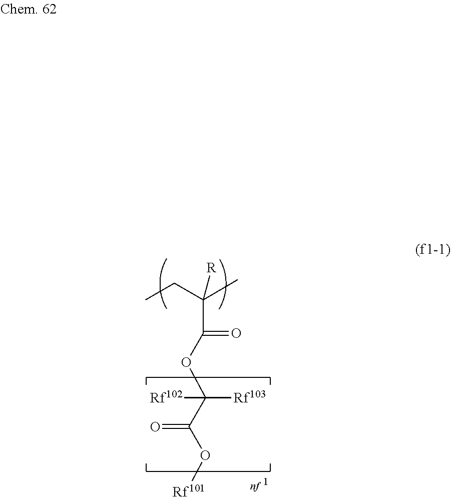

In the formula, R is the same as described above. Rf¹⁰² and Rf¹⁰³ each independently represent a hydrogen atom, a halogen atom, an alkyl group having 1 to 5 carbon atoms, or a halogenated alkyl group having 1 to 5 carbon atoms. Rf¹⁰² and Rf¹⁰³ may be the same as or different from each other. nf¹ is an integer of 0 to 5, and Rf¹⁰¹ is an organic group containing a fluorine atom.

In the general formula (f1-1), R bonded to a carbon atom at an α-position is the same as defined above. R is preferably a hydrogen atom or a methyl group.

Examples of the halogen atom of Rf¹⁰² and Rf¹⁰³ in the general formula (f1-1) include a fluorine atom, a chlorine atom, a bromine atom, and an iodine atom, and a fluorine atom is particularly preferred. Examples of the alkyl group having 1 to 5 carbon atoms for Rf¹⁰² and Rf¹⁰³ include those same as the alkyl group having 1 to 5 carbon atoms for the above R, and a methyl group or an ethyl group is preferred. Specific examples of the halogenated alkyl group having 1 to 5 carbon atoms for Rf¹⁰² and Rf¹⁰³ include a group in which a part or all of hydrogen atoms of the alkyl group having 1 to 5 carbon atoms are replaced by halogen atoms. Examples of the halogen atom include a fluorine atom, a chlorine atom, a bromine atom, and an iodine atom. A fluorine atom is particularly preferred. Among them, as Rf¹⁰² and Rf¹⁰³, a hydrogen atom, a fluorine atom, or an alkyl group having 1 to 5 carbon atoms is preferred, and a hydrogen atom, a fluorine atom, a methyl group, or an ethyl group is more preferred.

In the general formula (f1-1), n^{f1} is an integer of 1 to 5, preferably an integer of 1 to 3, and more preferably 1 or 2.

In the general formula (f1-1), Rf¹⁰¹ represents an organic group containing a fluorine atom, and is preferably a hydrocarbon group containing a fluorine atom.

The hydrocarbon group containing a fluorine atom may be any of a linear group, branched group, and cyclic group, and preferably has 1 to 20 carbon atoms, more preferably has 1 to 15 carbon atoms, and particularly preferably has 1 to 10 carbon atoms.

In the hydrocarbon group containing a fluorine atom, it is preferable that 25% or more of hydrogen atoms in the hydrocarbon group be fluorinated, it is more preferable that 50% or more of hydrogen atoms be fluorinated, and it is particularly preferable that 60% or more of hydrogen atoms be fluorinated, since hydrophobicity of the resist film during immersion exposure is enhanced.

Among them, Rf¹⁰¹ is more preferably a fluorinated hydrocarbon group having 1 to 6 carbon atoms, still more preferably a trifluoromethyl group, —CH₂—CF₃, —CH₂—CF₂—CF₃, —CH(CF₃)₂, —CH₂—CH₂—CF₃, or —CH₂—CH₂—CF₂—CF₂—CF₂—CF₃, and particularly preferably-CH₂—CF₃.

A weight average molecular weight (Mw) of the component (F) (based on polystyrene equivalent determined by gel permeation chromatography) is preferably 1000 to 50000, more preferably 5000 to 40000, and most preferably 10000 to 30000. When the weight average molecular weight is equal to or more than the lower limit value of the above range, the resist composition in the present embodiment has sufficient solubility in a resist solvent for use as a resist, and when the weight average molecular weight is equal to or less than the lower limit value of the above range, the resist composition in the present embodiment has good dry etching resistance and good cross-sectional shape of the resist pattern.

A dispersity (Mw/Mn) of the component (F) is preferably 1.0 to 5.0, more preferably 1.0 to 3.0, and most preferably 1.0 to 2.5.

In the resist composition of the present embodiment, the component (F) may be used alone or in combination of two or more kinds thereof.

When the resist composition contains the component (F), as a content of the component (F), the component (F) is generally used in a proportion of 0.5 parts by mass to 10 parts by mass with respect to 100 parts by mass of the component (A).

### <<Component (H)>>

The resist composition of the present embodiment may contain a surfactant component (hereinafter, referred to as “component (H)”) in order to improve the coatability, defoaming properties, leveling properties, and the like of the resist composition. The component (H) is not particularly limited and may be, for example, a silicon or acrylic surfactant. The component (H) may be either an ionic surfactant or a nonionic surfactant.

The silicon surfactant may be synthesized by a known method, or a commercially available product may be used. The silicon surfactant may contain a silane condensate having either or both of a urethane or urea bond. As the silicon surfactant, a silicone surfactant such as an unmodified silicone surfactant, a polyether-modified silicone surfactant, a polyester-modified silicone surfactant, an alkyl-modified silicone surfactant, an aralkyl-modified silicone surfactant, or a reactive silicone surfactant can be preferably used.

Specific examples of the commercially available silicone surfactant include PAINTAD M (manufactured by Dow Corning Toray Co., Ltd.), Topika K1000, Topika K2000, Topika K5000 (all manufactured by Takachiho Sangyo Co., Ltd.), XL-121 (a polyether-modified silicone surfactant manufactured by Clariant), and BYK-310 (a polyester-modified silicone-bed surfactant manufactured by BYK Chemie).

Examples of the acrylic surfactant include BYK-354 (manufactured by BYK Chemie).

Among them, a silicon surfactant is preferred.

In the resist composition of the present embodiment, the component (H) may be used alone or in combination of two or more kinds thereof.

When the resist composition contains the component (H), a content of the component (H) is generally 0.01 parts by mass to 3 parts by mass, preferably 0.01 parts by mass to 1 part by mass, and more preferably 0.01 parts by mass to 0.5 parts by mass, with respect to 100 parts by mass of the component (A).

### <Organic Solvent Component(S)>

The resist composition of the present embodiment can be produced by dissolving a resist material in an organic solvent component (hereinafter, referred to as “component(S)”).

As the component(S), those capable of dissolving components to be used to form a uniform solution may be used, and any one can be appropriately selected from those are known in the related art as a solvent for a chemically amplified resist composition.

In the resist composition of the present embodiment, the component(S) may be used alone or as a mixed solvent of two or more kinds thereof. Among them, PGMEA, PGME, γ-butyrolactone, EL, and cyclohexanone are preferred.

As the component(S), a mixed solvent of PGMEA and a polar solvent is also preferred. The blending ratio (mass ratio) may be appropriately determined in consideration of compatibility between PGMEA and the polar solvent.

As the component(S), a mixed solvent of γ-butyrolactone and at least one selected from PGMEA and EL is also preferred. In this case, as the mixing proportion, the mass ratio of the former to the latter is preferably 70:30 to 95:5.

An amount of the component(S) to be used is not particularly limited, and is appropriately set according to a coating film thickness at a concentration such that the component(S) can be applied to a substrate or the like. Generally, the component(S) is used such that a solid content concentration of the resist composition is in a range of 0.1 mass % to 20 mass %, and preferably 0.2 mass % to 15 mass %.

The resist composition in the present embodiment may further contain, if desired, compatible additives such as an additional resin, a dissolution inhibitor, a plasticizer, a stabilizer, a colorant, an antihalation agent, or a dye for improving the performance of the resist film.

The resist composition of the present embodiment contains the above-described component (A), component (B), and component (D), and, if necessary, the optional components described above.

For example, a resist composition containing the component (A), the component (B), the component (D), and the component (F) is suitable. Further, a resist composition containing the component (A), the component (B), the component (D), the component (F), and the component(S) is preferably exemplified.

It is preferred that the resist composition do not contain a compound that falls under the PFAS. The component contained in the resist composition preferably contains neither a compound having a trifluoromethyl group nor a compound having a difluoromethylene group. Further, the resist composition more preferably contains no fluorine atom.

As described above, the resist composition of the present embodiment contains the acid generating agent component (B) containing the above-described compound represented by the general formula (b1-1). It is presumed that, since in the compound represented by the general formula (b1-1), X₁ and Y₁ are not a fluorine atom, not only the environment burden can be reduced, but also segregation of the PAG anion on the resist pattern surface can be eliminated, and the resist composition of the present embodiment can form a resist pattern with a good shape and a resist pattern with excellent lithography properties. Further, the compound represented by the general formula (b1-1) contains a sulfonic acid group and a carboxylic acid group in the same structure. Therefore, due to the interaction between the sulfonic acid group and the carboxylic acid group in the same structure, the acid strength is increased (pKa is decreased), and a high sensitivity can be achieved.

### Method for Forming Resist Pattern

A method for forming a resist pattern according to another aspect of the present invention is a method including: a step of forming a resist film on a support using the resist composition of the embodiment; a step of exposing the resist film; and a step of developing the resist film to form a resist pattern.

One embodiment of the method for forming a resist pattern includes, for example, a method for forming a resist pattern performed as follows.

First, the resist composition of the embodiment is applied onto a support using a spinner or the like, and a baking treatment (a post apply bake (PAB)) is performed, for example, for 40 seconds to 120 seconds, preferably 50 seconds to 90 seconds under a temperature condition of 80° C. to 150° C. to form a resist film.

Next, the resist film is selectively exposed using an exposure apparatus such as an electron beam lithography apparatus or an EUV exposure apparatus, for example, by exposure through a mask (a mask pattern) on which a predetermined pattern is formed, or by exposure such as drawing by direct irradiation of an electron beam without through the mask pattern. Thereafter, a baking (a post-exposure bake (PEB)) treatment is performed, for example, for 40 seconds to 120 seconds, preferably 50 seconds to 90 seconds under a temperature condition of 80° C. to 150° C.

Next, the resist film is subjected to a developing treatment. The developing treatment is performed using an alkaline liquid developer in the case of an alkaline developing process, and using a liquid developer containing an organic solvent (an organic liquid developer) in the case of a solvent developing process.

After the developing treatment, a rinsing treatment is preferably performed. As the rinsing treatment, water rinsing using pure water is preferred in the case of the alkaline developing process, and a rinsing liquid containing an organic solvent is preferably used in the case of the solvent developing process.

In the case of the solvent developing process, after the developing treatment or the rinsing treatment, a process of removing a liquid developer or a rinsing liquid attached to the pattern by a supercritical fluid may be performed.

After the developing treatment or the rinsing treatment, drying is performed. In some cases, a baking treatment (a post bake) may be performed after the developing treatment.

Thus, a resist pattern can be formed.

The support is not particularly limited, and any related-art known supports can be used. Examples thereof include a substrate for electronic components or a substrate having a predetermined wiring pattern formed thereon. More specifically, a silicon wafer, a substrate made of a metal such as copper, chromium, iron, or aluminum, a glass substrate, or the like may be used. As a material of the wiring pattern, for example, copper, aluminum, nickel, or gold can be used.

A wavelength used for exposure is not particularly limited, and an ArF excimer laser, a KrF excimer laser, an F2 excimer laser, extreme ultraviolet (EUV), vacuum ultraviolet (VUV), electron beam (EB), and radiation rays such as X-rays and soft X-rays can be used. The resist composition is highly useful for a KrF excimer laser, an ArF excimer laser, EB or EUV.

A resist film exposure method may be general exposure (dry exposure) performed in an inert gas such as air or nitrogen, or may be liquid immersion lithography, and liquid immersion lithography is preferred.

The liquid immersion lithography is an exposure method in which a space between a resist film and a lowermost lens of an exposure apparatus is filled with a solvent (a liquid immersion medium) having a refractive index greater than that of air, and exposure (immersion exposure) is then performed in that state.

As the liquid immersion medium, a solvent having a refractive index larger than that of air and smaller than that of a resist film to be exposed is preferred. The refractive index of such a solvent is not particularly limited as long as it is within the range.

Examples of the solvent having a refractive index larger than that of air and smaller than that of the resist film include water, a fluorine inert liquid, a silicone solvent, and a hydrocarbon solvent.

Water is preferably used as the liquid immersion medium.

Examples of the alkaline liquid developer used in the developing treatment in the alkaline developing process include a 0.1 mass % to 10 mass % aqueous solution of tetramethylammonium hydroxide (TMAH).

The organic solvent contained in the organic liquid developer used in the developing treatment in the solvent developing process may be any solvents capable of dissolving the component (A) (the component (A) before exposure), and may be appropriately selected from known organic solvents. Specific examples thereof include polar solvents such as a ketone solvent, an ester solvent, an alcohol solvent, a nitrile solvent, an amide solvent, and an ether solvent, and hydrocarbon solvents.

Examples of the ester solvent include methyl acetate, butyl acetate, ethyl acetate, isopropyl acetate, pentyl acetate, isopentyl acetate, amyl acetate, propylene glycol monomethyl ether acetate, ethylene glycol monoethyl ether acetate, diethylene glycol monobutyl ether acetate, diethylene glycol monoethyl ether acetate, ethyl-3-ethoxypropionate, 3-methoxybutyl acetate, 3-methyl-3-methoxybutyl acetate, methyl formate, ethyl formate, butyl formate, propyl formate, ethyl lactate, butyl lactate, propyl lactate, butyl butanoate, methyl 2-hydroxyisobutyrate, isoamyl acetate, isobutyl isobutyrate, and butyl propionate.

Examples of the nitrile solvent include acetonitrile, propionitrile, valeronitrile, and butyrnitrile.

If necessary, known additives may be added to the organic liquid developer. Examples of the additive include a surfactant. The surfactant is not particularly limited, and for example, ionic or nonionic fluorine and/or silicone surfactants can be used.

The developing treatment can be performed by a known developing method.

Examples thereof include a method in which a support is immersed with respect to a liquid developer for a certain period of time (a dip method), a method in which a liquid developer is piled up on a surface of a support by surface tension and left standing for a certain period of time (a paddle method), a method in which a liquid developer is sprayed onto a surface of a support (a spray method), a method in which a liquid developer is continuously dispensed by scanning a liquid developer dispense nozzle at a constant speed onto a support that is rotating at a constant speed (a dynamic dispense method).

The rinsing treatment (a washing treatment) using the rinsing liquid can be performed by a known rinsing method. Examples of the method of the rinsing treatment include a method in which a rinsing liquid is continuously dispensed onto a support that is rotating at a constant speed (a spin dispensing method), a method in which a support is immersed in a rinsing liquid for a certain period of time (a dip method), and a method in which a rinsing liquid is sprayed onto a surface of a support (a spray method).

Various materials used in the resist composition of the embodiment and the method for forming a pattern of the embodiment (for example, a resist solvent, a liquid developer, a rinsing liquid, a composition for forming an antireflection film, a composition for forming a top coat, and the like) preferably does not contain impurities such as a metal, a metal salt containing a halogen, an acid, an alkaline, and a component containing a sulfur atom or a phosphorus atom. Here, examples of the impurity containing a metal atom include Na, K, Ca, Fe, Cu, Mn, Mg, Al, Cr, Ni, Zn, Ag, Sn, Pb, Li, and salts thereof. A content of the impurities contained in these materials is preferably 200 ppb or less, more preferably 1 ppb or less, still more preferably 100 ppt (parts per trillion) or less, and particularly preferably 10 ppt or less, and it is most preferable that the impurity be not substantially contained (equal to or less than a detection limit of a measuring device).

In the method for forming a resist pattern of the present embodiment described above, since the resist composition according to the present embodiment described above is used, a high sensitivity can be achieved, and a resist pattern with excellent lithography properties and a good shape can be formed when forming a resist pattern.

### Compound

A compound according to a third embodiment of the present invention is a compound represented by the following general formula (b1-1).

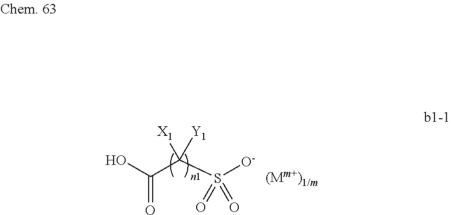

In the general formula (b1-1), X₁ and Y₁ each independently represent a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group. X₁ and Y₁ may be bonded to each other to form a ring; n1 represents an integer of 1 to 10. m is an integer of 1 or more. M^{m+} represents an m-valent organic cation.

The compound represented by the general formula (b1-1) is the same as the component (b1-1) in the resist composition according to the embodiment of the present invention, and preferred examples thereof are also the same.

Method for Producing Compound Represented by General Formula (b1-1)

The compound represented by the general formula (b1-1) can be produced by using a known method.

For example, the compound represented by the general formula (b1-1) can be obtained by performing a salt exchange reaction between a precursor Bpre represented by the following general formula (Bpre) and a compound S—O represented by the following general formula (S-0).

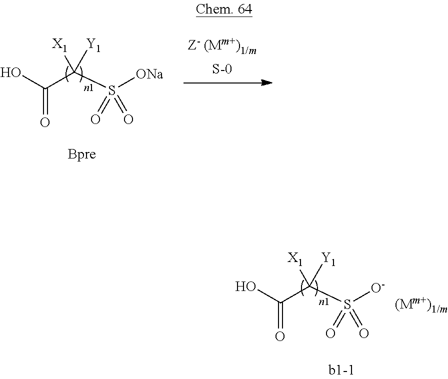

In the formula, X₁ and Y₁ each independently represent a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group. X₁ and Y₁ may be bonded to each other to form a ring. n1 represents an integer of 1 to 10. m is an integer of 1 or more. M^{m+} represents an m-valent organic cation. Z⁻ is a non-nucleophilic ion.

More specifically, the salt exchange reaction is a step of reacting a precursor Bpre with a compound S—O for salt exchange in a solvent such as water, dichloromethane, acetonitrile, or chloroform to exchange a cation (Na) of the precursor Bpre with a cation of the compound S-0, thereby obtaining the compound represented by the general formula (b1-1).

In the above formula, Z⁻ is, for example, an ion that can become an acid having an acidity lower than that of the precursor Bpre, and specific examples thereof include halogen ions such as a bromide ion and a chloride ion, BF₄⁻, AsF₆⁻, SbF₆⁻, PF₆⁻, and ClO₄⁻.

A reaction temperature for the salt exchange reaction is, for example, 0° C. to 100° C., and a reaction time is, for example, 10 minutes to 24 hours.

After the salt exchange reaction is completed, the compound in the reaction liquid may be isolated and purified. For isolation and purification, a known method in related art can be used. For example, a suitable combination of concentration, solvent extraction, distillation, crystallization, recrystallization, chromatography, and the like can be used.

The structure of the compound obtained as described above can be identified by general organic analysis methods such as 1H-nuclear magnetic resonance (NMR) spectrometry, 13C-NMR spectrometry, 19F-NMR spectrometry, infrared absorption (IR) spectrometry, mass spectrometry (MS), elemental analysis, and X-ray diffraction.

The raw materials used in each step may be commercially available products or may be synthesized by a known method.

### Acid Generating Agent

An acid generating agent according to a fourth embodiment of the present invention includes the above compound according to the third embodiment.

Such an acid generating agent is useful as an acid generating agent component for a chemically amplified resist composition. By using such an acid generating agent component in a chemically amplified resist composition, in the formation of a resist pattern, an environmental burden can be reduced, a sensitivity can be increased, and a resist pattern having excellent lithography properties can be formed.

### EXAMPLES

The present invention will be described in more detail below with reference to Examples, but the present invention is not limited to these Examples.

### <Synthesis Example of Compound B-2>

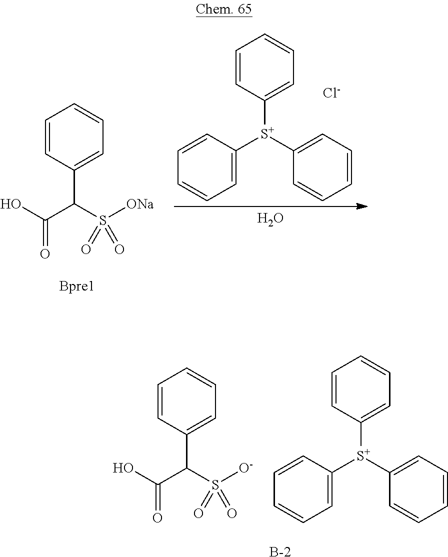

Under a nitrogen atmosphere, a precursor compound Bpre1 (11.5 g, 48.2 mmol, 1 equiv.), ion-exchanged water (58 mL), and triphenylmethylsulfonium chloride (15.1 g, 50.6 mmol, 1.05 equiv.) were sequentially added to a 100 mL eggplant flask, followed by stirring for 2 hours. The reaction solution was extracted with methylene chloride, and the obtained organic layer was washed with ion-exchanged water. Magnesium sulfate was added to the organic layer, which was then filtered, and the filtrate was concentrated. After concentration, the obtained solid was dried under reduced pressure at 50° C. for 16 hours to obtain 18.8 g of compound B-2 as a white solid.

### <Synthesis of Compounds B-1, B-3 to B-8, B-10>

Compounds B-1, B-3 to B-8, and B-10 were obtained in the same manner as in the above “Synthesis Example of Compound B-2” by changing the above precursor compound and the salt exchange compound.

The obtained compounds were each subjected to NMR measurement, and structures thereof were identified from the following analytical results.

TABLE 1

| Component | | | | | | |
|---|---|---|---|---|---|---|
| B | Structure | ¹H-NMR(DMSO-d6, 400 MHz): δ (ppm) | | | | |
| B-1 | 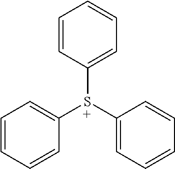 | 12.2 (s,—OH,1H) | 7.3~7.5 (m,—Ar,15H) | 4.4 (s,—CH,2H) | | |
| | 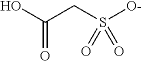 | | | | | |
| B-2 | 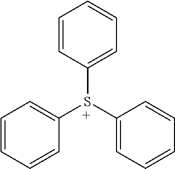 | 12.3 (s,—OH,1H) | 7.7~7.8 (m,—Ar,15H) | 7.5 (m,—Ar,2H) | 7.2 (m,—Ar,3H) | 4.6 (s,—CH,1H) |
| | 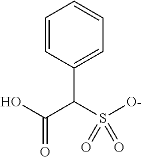 [SMILES: O=C(O)C(c1ccccc1)S(=O)(=O)[O-]] | | | | | |
| B-3 | 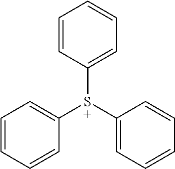 | 12.2 (s,—OH,1H) | 7.3~7.4 (m,—Ar,15H) | | | |
| | 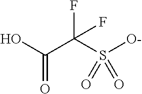 | | | | | |
| B-4 | 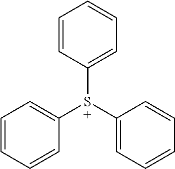 | 7.3~7.4 (m,—Ar,15H) | 4.2 (s,—OH,1H) | 4.1 (t,—CH,2H) | 3.6 (t,—CH,2H) | |
| | 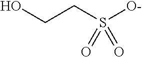 | | | | | |
| B-5 | 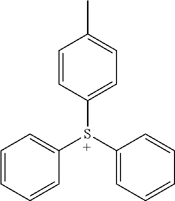 | 12.2 (s,—OH,1H) | 7.3~7.4 (m,—Ar,10H) | 7.1~7.3 (m,—Ar,4H) | 4.4 (s,—CH,2H) | 2.4 (s,—CH,3H) |
| | 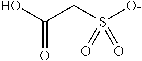 | | | | | |
| B-6 | 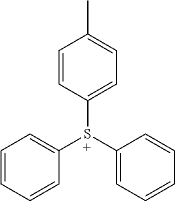 | 12.7 (s,—OH,1H) | 7.3~7.4 (m,—Ar,15H) | 7.1~7.3 (m,—Ar,4H) | 5.2 (s,—CH,1H) | 2.4 (s,—CH,3H) |
| | 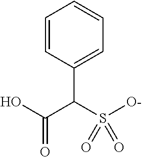 [SMILES: O=C(O)C(c1ccccc1)S(=O)(=O)[O-]] | | | | | |
| B-7 | 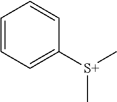 | 12.2 (s,—OH,1H) | 7.3~7.4 (m,—Ar,5H) | 4.4 (s,—CH,2H) | 3.3 (s,—CH,6H) | |
| | 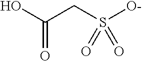 | | | | | |
| B-8 | 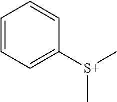 | 12.7 (s,—OH,1H) | 7.3~7.4 (m,—Ar,10H) | 5.2 (s,—CH,1H) | 3.3 (s,—CH,6H) | |
| | 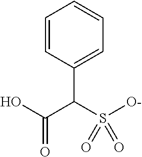 [SMILES: O=C(O)C(c1ccccc1)S(=O)(=O)[O-]] | | | | | |
| B-9 | 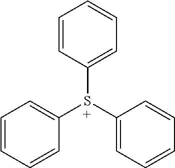 | 7.7~7.8 (m,—Ar,15H) | 1.3-2.9 (m,—CH or —CH2,9H) | 0.7, 1.1 (s,—CH,6H) | | |
| | 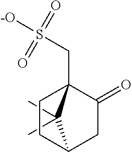 | | | | | |
| B-10 | 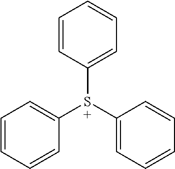 | 12.8 (s,—OH,1H) | 8.9~8.7 (m,—Ar, 15H) | 3.1 (d,—CH,1H) | 2.1~0.9 (m,—CH or —CH2,11H) | |
| | 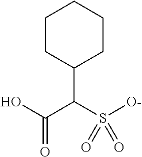 [SMILES: O=C(O)C(C1CCCCC1)S(=O)(=O)[O-]] | | | | | |

### <Preparation of Resist Composition> (Examples 1 to 12, and Comparative Examples 1 to 5)

Components shown in Table 2 were mixed and dissolved to prepare resist compositions of each Example.

TABLE 2

| | | | Component | | | | | |
|---|---|---|---|---|---|---|---|---|
| | | Component | (D) | | | | | |
| | | (B) | acid- | | | | | Shape |
| | Component | acid | diffusion | Component | Component | | | top |
| | (A) | generating | controlling | (H) | (S) | Eop | E.L. | CD/bottom |
| | resin | agent | agent | surfactant | solvent | [mJ/cm²] | [%] | CD |
| Example 1 | A-1[100] | B-2[4.8] | D-1[4.0] | — | S-1[2910.0] | 45.0 | 9.0 | 0.73 |
| Example 2 | A-1[100] | B-6[4.9] | D-1[4.0] | — | S-1[2920.0] | 47.2 | 8.6 | 0.72 |
| Example 3 | A-1[100] | B-8[3.6] | D-1[4.0] | — | S-1[2880.0] | 64.0 | 7.0 | 0.75 |
| Example 4 | A-1[100] | B-1[4.0] | D-1[4.0] | — | S-1[2890.0] | 43.0 | 8.0 | 0.78 |
| Example 5 | A-1[100] | B-5[4.2] | D-1[4.0] | — | S-1[2900.0]] | 45.1 | 7.6 | 0.78 |
| Example 6 | A-1[100] | B-7[2.8] | D-1[4.0] | — | S-1[2860.0] | 61.2 | 7.4 | 0.76 |
| Example 7 | A-3[100] | B-2[4.8] | D-1[4.0] | — | S-1[2910.0] | 38.0 | 9.1 | 0.74 |
| Example 8 | A-3[100] | B-1[4.0] | D-1[4.0] | — | S-1[2910.0] | 36.3 | 9.5 | 0.84 |
| Example 9 | A-2[100] | B-2[6.4] | D-1[8.0] | — | S-1[648.5] | 74.0 | — | 0.7 |
| Example 10 | A-2[100] | B-1[5.4] | D-1[8.0] | — | S-1[642.5] | 70.7 | — | 0.76 |
| Example 11 | A-2[100] | B-10[6.4] | D-1[8.0] | — | S-1[648.5] | 73.0 | — | 0.69 |
| Example 12 | A-2[100] | B-10[6.4] | D-1[8.0] | H-1 | S-1[648.5] | 73.0 | — | 0.69 |
| | | | | [0.08] | | | | |
| Comparative | A-1[100] | B-3[4.4] | D-1[4.0] | — | S-1[2900.0] | 32.0 | 10.8 | 0.62 |
| Example 1 | | | | | | | | |
| Comparative | A-1[100] | B-4[3.9] | D-1[4.0] | — | S-1[2890.0] | 67.0 | 4.6 | 0.56 |
| Example 2 | | | | | | | | |
| Comparative | A-1[100] | B-9[5.0] | D-1[4.0] | — | S-1[2920.0] | 80< (poor | — | — |
| Example 3 | | | | | | resolution) | | |
| Comparative | A-2[100] | B-3[5.9] | D-1[8.0] | — | S-1[645.0] | 52.6 | — | 0.65 |
| Example 4 | | | | | | | | |
| Comparative | A-2[100] | B-4[5.2] | D-1[8.0] | — | S-1[641.5] | 110.2 | 0.0 | 0.49 |
| Example 5 | | | | | | | | |

In Table 2, abbreviations have the following meanings. The numerical values in [ ] are blending amounts (parts by mass).

A-1: a macromolecular compound represented by the following chemical formula A-1. A weight average molecular weight (Mw) of a macromolecular compound A-1, calculated based on standard polystyrene, as determined by GPC measurement, was 7,000, and a molecular weight dispersity (Mw/Mn) thereof was 1.60. A copolymer composition ratio (a proportion (a molar ratio) of each structural unit in the structural formula) determined by ¹³C-NMR was l/m/n=40/40/20.

A-2: a macromolecular compound represented by the following chemical formula A-2. A weight average molecular weight (Mw) of a macromolecular compound A-2, calculated based on standard polystyrene, as determined by GPC measurement, was 10,000, and a molecular weight dispersity (Mw/Mn) thereof was 2.07. A copolymer composition ratio (a proportion (a molar ratio) of each structural unit in the structural formula) determined by ¹³C-NMR was l/m/n=60/15/25.

A-3: a macromolecular compound represented by the following chemical formula A-3. A weight average molecular weight (Mw) of a macromolecular compound A-3, calculated based on standard polystyrene, as determined by GPC measurement, was 7,000, and a molecular weight dispersity (Mw/Mn) thereof was 1.56. A copolymer composition ratio (a proportion (a molar ratio) of each structural unit in the structural formula) determined by ¹³C-NMR was l/m/n=50/50.

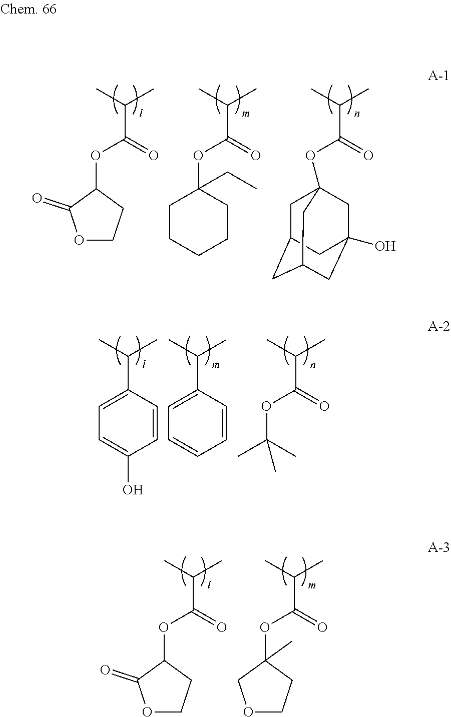

B-1 to B-10: acid generating agents composed of the above compounds B-1 to B-10, respectively

D-1: an acid diffusion control agent composed of a compound represented by the following chemical formula D-1.

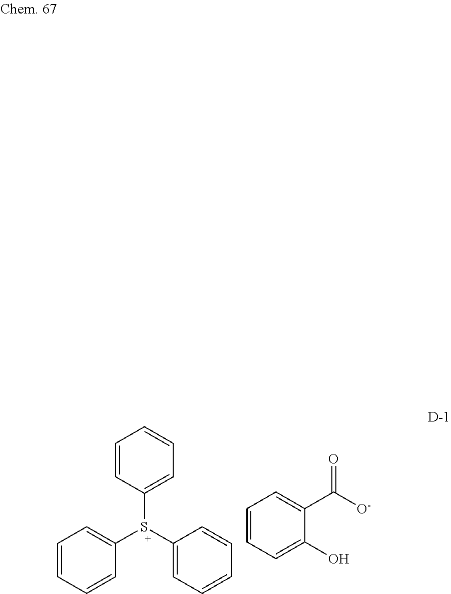

H-1: a silicone surfactant (a silicone chain-containing polymer obtained by polymerization reaction of polypropylene glycol-polybutylene glycol-monomethacrylate (the average number of repeats of propylene glycol was 1, and the average number of repeats of butylene glycol was 6) with a monomethacrylate compound having a polysiloxane bond represented by the following formula (the number average of x2 was 10) (synthesized by a method described in Synthesis Example 1 in WO2023/140036).

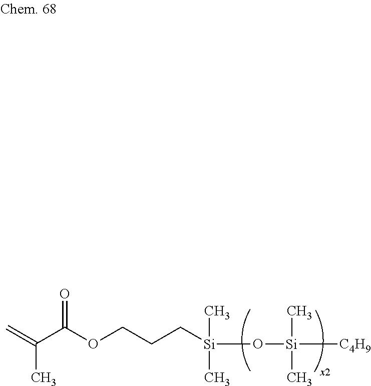

S-1: a mixed solvent of 45 mass % of propylene glycol monomethyl ether acetate, 30 mass % of propylene glycol monomethyl ether, and 25 mass % of cyclohexanone

### Lithography Evaluation 1 (ArF Evaluation)

For Examples 1 to 8 and Comparative Examples 1 to 3, KrF evaluations (resist pattern formation 1, optimal exposure amount (Eop) evaluation 1, Exposure Latitude (E.L.) evaluation 1, and LS pattern shape evaluation 1) were performed.

### <Resist Pattern Formation 1>

An organic antireflection film composition “ARC95” (manufactured by Brewer Science, Inc.) was applied onto a 12-inch silicon wafer using a spinner, and the composition was then baked and dried on a hotplate at 205° C. for 60 seconds to form an organic antireflection film having a film thickness of 90 nm.

The resist composition was applied onto the formed antireflection film using a spinner, and was then prebaked (PAB) on a hotplate at 110° C. for 60 seconds and dried to form a resist film having a film thickness of 100 nm.

Next, the resist film was selectively irradiated with an ArF excimer laser (193 nm) through a photomask (a binary mask) using an ArF exposure apparatus NSR—S308F [manufactured by Nikon Corporation; numerical aperture (NA)=0.92, Sigma=0.95]. Thereafter, a PEB treatment was performed at 110° C. for 60 seconds.

Next, an alkaline development was performed at 23° C. for 30 seconds using a 2.38 mass % aqueous TMAH solution (product name: NMD-3, manufactured by TOKYO OHKA KOGYO CO., LTD.). Thereafter, water rinsing was performed for 30 seconds using pure water, and drying was performed by shaking.

As a result, a 1:1 line-and-space (LS) pattern having a line width of 100 nm and a pitch of 200 nm was formed in each of Examples.

### Optimal Exposure Amount (Eop) Evaluation 1

A line size in the above <Resist Pattern Formation 1> was observed, and an exposure amount at which an LS pattern having a line width of 100 nm and a pitch of 200 nm was formed was shown in Table 2 as an optimal exposure amount (mJ/cm²).

### Exposure Latitude (E.L.) Evaluation 1

An exposure amount was determined when lines of the LS pattern having a line width of 100 nm and a pitch of 200 nm were formed within a range of +5% (95 nm to 105 nm) of a target dimension (line width 100 nm) by the above <Resist Pattern Formation 1>, and EL (unit: %) was calculated by the following formula.

$$5 \% EL ( \% ) = ( \vert E 1 - E 2 \vert / Eop ) \times 100$$

E1 indicates an exposure amount (mJ/cm²) when an LS pattern having a line width of 95 nm is formed, and E2 indicates an exposure amount (mJ/cm²) when an LS pattern having a line width of 105 nm is formed.

### LS Pattern Shape Evaluation 1

A shape of the LS pattern formed by the above <Resist Pattern Formation 1> was observed using a length measuring SEM (scanning electron microscope, accelerating voltage 800 V, product name: SU-8000, manufactured by Hitachi High-Technologies Corporation), and Top CD and Bottom CD were measured (N=3, average value). A ratio of Top CD to Bottom CD was calculated and results thereof were shown in Table 2 as “shape.” The closer the Top/Bottom is to “1”, the better the shape is.

### Lithography Evaluation 2 (KrF Evaluation)

For Example 9, Example 10, Comparative Example 4, and Comparative Example 5, KrF evaluations (resist pattern formation 2, optimal exposure amount (Eop) evaluation 2, LS pattern shape evaluation 2) were performed.

### <Resist Pattern Formation 2>

An organic antireflection film composition “DUV-42P” (manufactured by Brewer Science, Inc.) was applied onto a 6-inch silicon wafer using a spinner, and the composition was then baked and dried on a hotplate at 205° C. for 60 seconds to form an organic antireflection film having a film thickness of 60 nm.

A resist composition was applied onto the antireflection film using a spinner, pre-baked (PAB) on a hot plate at 110° C. for 60 seconds, and dried to form a resist film having a film thickness of 420 nm.

The resist film was selectively irradiated with a KrF excimer laser (248 nm) through a photomask (a binary mask) using a KrF exposure apparatus NSR-S203B [manufactured by Nikon Corporation; numerical aperture (NA)=0.68, Sigma=0.75]. Thereafter, a PEB treatment was performed at 110° C. for 60 seconds.

Next, an alkaline development was performed at 23° C. for 60 seconds using a 2.38 mass % TMAH aqueous solution (trade name: NMD-3, manufactured by Tokyo Ohka Kogyo Co., Ltd.), and then water rinsing was performed for 30 seconds using pure water, followed by shaking and drying.

As a result, a 1:1 line-and-space (LS) pattern having a line width of 170 nm and a pitch of 340 nm was formed in each of Examples.

### Optimal Exposure Amount (Eop) Evaluation 2

A line size in the above <Resist Pattern Formation 2> was observed, and an exposure amount at which the LS pattern having a line width of 170 nm and a pitch of 340 nm was formed was shown in Table 2 as an optimal exposure amount (mJ/cm²).

### LS Pattern Shape Evaluation 2

A shape of the LS pattern formed by the above <Resist Pattern Formation 2> was observed using a length measuring SEM (scanning electron microscope, accelerating voltage 800 V, product name: SU-8000, manufactured by Hitachi High-Technologies Corporation), and Top CD and Bottom CD were measured (N=3, average value). A ratio of Top CD to Bottom CD was calculated and results thereof were shown in Table 2 as “shape.” The closer the Top/Bottom is to “1”, the better the shape is.

From the results shown in Table 2, it can be confirmed that, according to the resist composition in Example to which the present invention is applied, a high sensitivity can be achieved in the formation of a resist pattern, and a resist pattern having excellent lithography properties and a good shape can be formed.

### Lithography Evaluation 3 (KrF Evaluation)

For Example 11 and Example 12, KrF evaluations (resist pattern formation 3, optimal exposure amount (Eop) evaluation, and LS pattern shape evaluation 3) were performed.

### <Resist Pattern Formation 3>

The resist compositions in Examples 11 and 12 were each applied onto a 6-inch silicon wafer that was subjected to an HMDS treatment using a spinner, pre-baked (PAB) on a hot plate at 110° C. for 60 seconds, and dried to form resist films having a film thickness of 1.7 μm.

The resist film was selectively irradiated with a KrF excimer laser (248 nm) through a photomask (a binary mask) using a KrF exposure apparatus NSR—S205C [manufactured by Nikon Corporation; numerical aperture (NA)=0.6, Sigma=0.5]. Thereafter, a PEB treatment was performed at 110° C. for 60 seconds.

Next, an alkaline development was performed at 23° C. for 60 seconds using a 2.38 mass % TMAH aqueous solution (trade name: NMD-3, manufactured by Tokyo Ohka Kogyo Co., Ltd.), and then water rinsing was performed for 30 seconds using pure water, followed by shaking and drying.

As a result, a 1:1 line-and-space (LS) pattern having a line width of 400 nm and a pitch of 800 nm was formed in each of Examples.

### Optimal Exposure Amount (Eop) Evaluation 3

A line size in the above <Resist Pattern Formation 3> was observed, and an exposure amount at which the LS pattern having a line width of 400 nm and a pitch of 800 nm was formed was shown in Table 2 as an optimal exposure amount (mJ/cm²).

### LS Pattern Shape Evaluation 3

A shape of the LS pattern formed by the above <Resist Pattern Formation 2> was observed using a length measuring SEM (scanning electron microscope, accelerating voltage 800 V, product name: SU-8000, manufactured by Hitachi High-Technologies Corporation), and Top CD and Bottom CD were measured (N=3, average value). A ratio of Top CD to Bottom CD was calculated and results thereof were shown in Table 2 as “shape.” The closer the Top/Bottom is to “1”, the better the shape is.

From the results shown in Table 2, it can be confirmed that, according to the resist composition in Example to which the present invention is applied, even when a resist film having a thickness of 1 μm or more is formed, a resist pattern having excellent lithography properties can be formed.

Although the present invention has been described in detail with reference to specific embodiments, it is apparent to those skilled in the art that various changes and modifications can be made without departing from the spirit and scope of the present invention. The present application is based on a Japanese patent application (JP2023-192592) filed on Nov. 10, 2023 and a Japanese patent application (JP2024-085115) filed on May 24, 2024, the disclosure of which is hereby incorporated by reference herein in its entirety.

What is claimed is:

1. A resist composition that generates an acid upon exposure and whose solubility with respect to a liquid developer changes by an action of the acid, the resist composition comprising:
a base material component (A) whose solubility with respect to a liquid developer changes by an action of an acid;
an acid generating agent component (B) that generates an acid upon exposure; and
an acid diffusion control agent component (D), wherein
the acid generating agent component (B) comprises a compound represented by the following general formula (b1-1): 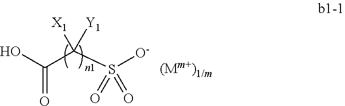
where X₁ and Y₁ each independently represents a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group; X₁ and Y₁ may be bonded to each other to form a ring; n1 represents an integer of 1 to 10; m is an integer of 1 or more; and M^{m+} represents an m-valent organic cation.

2. The resist composition according to claim 1, wherein the compound represented by the general formula (b1-1) is a compound represented by the following general formula (b1-2): 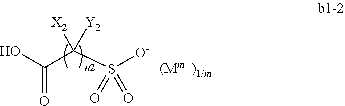
where X₂ and Y₂ each independently represents a hydrogen atom, an aliphatic hydrocarbon group, or an aromatic hydrocarbon group; n2 represents an integer of 1 or 2; m is an integer of 1 or more; and M^{m+} represents an m-valent organic cation.

3. The resist composition according to claim 1, wherein the compound represented by the general formula (b1-1) is a compound represented by the following general formula (b1-3): 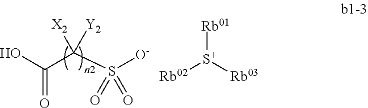
where X₂ and Y₂ each independently represents a hydrogen atom, an aliphatic hydrocarbon group, or an aromatic hydrocarbon group; n2 represents an integer of 1 or 2; Rb⁰¹ to Rb⁰³ each independently represents an aryl group, an alkyl group, or an alkenyl group; the aryl group, the alkyl group, and the alkenyl group may each independently have one or more substituents selected from the group consisting of an alkyl group, an aldehyde group, an acyl group, a hydroxyl group, and a halogen atom as a substituent; and Rb⁰¹ to Rb⁰³ may be bonded to each other to form a ring together with a sulfur atom in the general formula (b1-3).

4. The resist composition according to claim 1, wherein the base material component (A) comprises a macromolecular compound (A1) comprising a structural unit (a1) represented by the following general formula (a1-1): 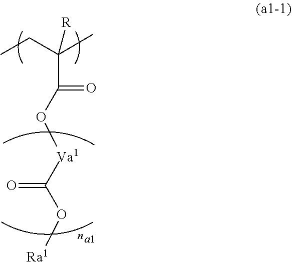
where R represents a hydrogen atom, an alkyl group having 1 to 5 carbon atoms, or a halogenated alkyl group having 1 to 5 carbon atoms; Va¹ represents a divalent hydrocarbon group which may have an ether bond; nₐ₁ is an integer of 0 to 2; and Ra¹ represents an acid-dissociable group.

5. The resist composition according to claim 1, wherein the acid diffusion control agent component (D) comprises a compound represented by any one of the following general formulae (d1-1) to (d1-3): 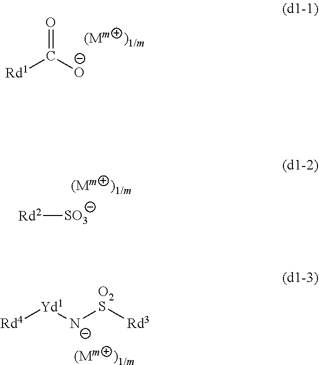
where Rd¹ to Rd⁴ represent a cyclic group that may include a substituent, a chained alkyl group that may include a substituent, or a chained alkenyl group that may include a substituent; and in Rd² in the formula (d1-2), no fluorine atom is bonded to a carbon atom adjacent to the S atom; Yd¹ represents a single bond or a divalent linking group; m represents an integer of 1 or more; and M^{m+}'s each independently represents an m-valent organic cation.

6. A method for forming a resist pattern comprising:
forming a resist film on a support using the resist composition according to claim 1;
exposing the resist film; and
developing the resist film to form a resist pattern.

7. A compound represented by the following general formula (b1-1): 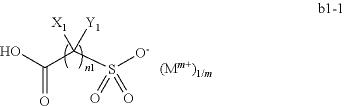
where X₁ and Y₁ each independently represents a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group; X₁ and Y₁ may be bonded to each other to form a ring; n1 represents an integer of 1 to 10; m is an integer of 1 or more; and M^{m+} represents an m-valent organic cation.

8. An acid generating agent comprising:
a compound represented by the following general formula (b1-1): 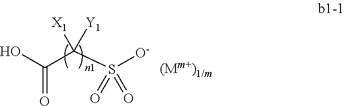
where X₁ and Y₁ each independently represents a halogen atom other than a fluorine atom, a hydrogen atom, an aliphatic hydrocarbon group, an aromatic hydrocarbon group, a heterocyclic group, an electron-withdrawing group, a selenol group, a pentafluorosulfanyl group, a mercapto group, a thiocarboxylic acid group, or a dithiocarboxylic acid group; X₁ and Y₁ may be bonded to each other to form a ring; n1 represents an integer of 1 to 10; m is an integer of 1 or more; and M^{m+} represents an m-valent organic cation.
